(19) United States
(12) Patent Application Publication
(10) Pub. No.: US 2022/0220144 A1
(43) Pub. Date: Jul. 14, 2022

(54) MULTIMERIC PAN-SELECTIN ANTAGONISTS

(71) Applicant: GLYCOMIMETICS, INC., Rockville, MD (US)

(72) Inventors: John L. MAGNANI, Gaithersburg, MD (US); John M. PETERSON, Slate Hill, NY (US)

(73) Assignee: GLYCOMIMETICS, INC., Rockville, MD (US)

(21) Appl. No.: 17/605,105

(22) PCT Filed: Apr. 21, 2020
(86) PCT No.: PCT/US2020/029070

§ 371 Date: Oct. 20, 2021

Related U.S. Application Data
(60) Provisional application No. 62/838,218, filed on Apr. 24, 2019.

(51) Int. Cl. C07H 15/26; C07H 15/207; C07H 15/22

(52) U.S. Cl. CPC C07H 15/26; C07H 15/22; C07H 15/207

(57) ABSTRACT

Compounds, compositions, and methods for modulating in vitro and in vivo processes mediated by selectin binding are described herein. For example, multimeric selectin modulators and their use are described, wherein the multimeric selectin modulators comprise a glycomimetic linked to a member of a class of compounds termed BASAs (Benzyl Amino Sulfonic Acids) or a member of a class of compounds termed BACAs (Benzyl Amino Carboxylic Acids).

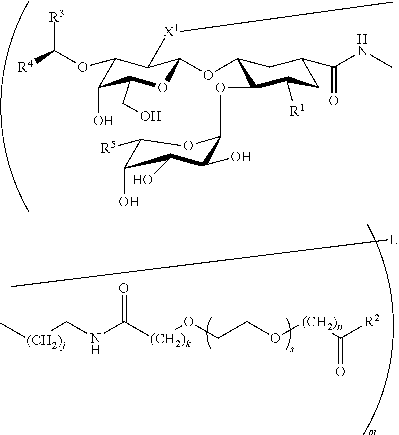

This application claims the benefit under 35 U.S.C. § 119(e) to U.S. Provisional Application No. 62/838,218 filed Apr. 24, 2019, which application is incorporated by reference herein in its entirety.

Compounds, compositions, and methods for modulating in vitro and in vivo processes mediated by selectin binding are described herein. For example, multimeric selectin modulators and their uses are described, wherein the multimeric selectin modulators comprise a glycomimetic linked to a member of a class of compounds termed BASAs (Benzyl Amino Sulfonic Acids) or a member of a class of compounds termed BACAs (Benzyl Amino Carboxylic Acids).

When a tissue is infected or damaged, the inflammatory process directs leukocytes and other immune system components to the site of infection or injury. Within this process, leukocytes play an important role in the engulfment and digestion of microorganisms. The recruitment of leukocytes to infected or damaged tissue is critical for mounting an effective immune defense.

Selectins are a group of structurally similar cell surface receptors important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells and binds to the carbohydrate sialyl-Lewis^{x} (SLe^{x}) which is presented as a glycoprotein or glycolipid on the surface of certain leukocytes (monocytes and neutrophils) and helps these cells adhere to capillary walls in areas where surrounding tissue is infected or damaged. E-selectin also binds to sialyl-Lewis^{a} (SLe^{a}) which is expressed on many tumor cells. P-selectin is expressed on inflamed endothelium and platelets and also recognizes SLe^{x} and SLe^{a} but also contains a second site that interacts with sulfated tyrosine. The expression of E-selectin and P-selectin is generally increased when the tissue adjacent to a capillary which is infected or damaged. L-selectin is expressed on leukocytes. Selectin-mediated intercellular adhesion and formation of new capillaries during angiogenesis are examples of selectin-mediated functions.

Although selectin-mediated cell adhesion is required for fighting infection and destroying foreign material, there are situations in which cell adhesion is undesirable or excessive resulting in tissue damage instead of repair. For example, pathologies that involve abnormal adhesion of white blood cells include autoimmune and inflammatory diseases, shock, and reperfusion injuries. Abnormal cell adhesion may also play a role in transplant and graft rejection. In addition, some circulating cancer cells appear to take advantage of the inflammatory mechanism to bind to activated endothelium. In these circumstances, modulation of selectin-mediated intercellular adhesion may be desirable.

Accordingly, there is a need in the art for identifying modulators, e.g., inhibitors, of selectin-mediated function, e.g., of selectin-dependent cell adhesion, and for the development of methods employing such compounds to inhibit conditions associated with excessive selectin activity. The present disclosure may fulfill one or more of these needs and/or may provide other advantages. For example, the compounds of the present disclosure may be highly potent E-selectin and/or P-selectin antagonists.

Briefly stated, compounds, compositions, and methods for modulating selectin-mediated processes are disclosed. The compounds comprise multimeric glycomimetics linked to a member of a class of compounds termed BASAs or to a member of a class of compounds termed BACAs. The compounds may be combined with at least one additional pharmaceutically acceptable ingredient to form a pharmaceutical composition. The compounds or compositions may be used in a method to modulate (e.g., inhibit or enhance) a selectin-mediated function, such as inhibiting a selectin-mediated intercellular adhesion.

Disclosed are multimeric selectin-modulators of Formula (I):

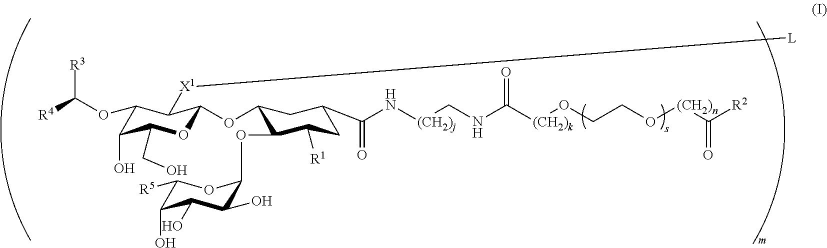

prodrugs thereof, and pharmaceutically acceptable salts of any of the foregoing, wherein R¹, R², R³, R⁴, R⁵, X¹, L, j, k, n, s, and m are defined herein.

As used herein, “compound of Formula (I)” includes multimeric selectin-modulators of Formula (I), pharmaceutically acceptable salts of multimeric selectin-modulators of Formula (I), prodrugs of multimeric selectin-modulators of Formula (I), and pharmaceutically acceptable salts of prodrugs of multimeric selectin-modulators of Formula (I).

In some embodiments, pharmaceutical compositions comprising at least one compound of Formula (I) and optionally at least one additional pharmaceutically acceptable ingredient are presented.

In some embodiments, a compound of Formula (I) and/or a pharmaceutical composition comprising at least one compound of Formula (I) may be used for the preparation and/or manufacture of a medicament for use in treating at least one of the diseases, disorders, and conditions described herein.

In some embodiments, a method for modulating a selectin-mediated function comprising administering to a subject in need thereof an effective amount of at least one compound of Formula (I) and/or a pharmaceutical composition comprising at least one compound of Formula (I) and optionally at least one additional pharmaceutically acceptable ingredient is disclosed. In some embodiments, the selectin-mediated function is enhanced. In some embodiments, the selectin-mediated function is inhibited.

In some embodiments, a method for contacting a cell expressing a selectin to modulate (e.g., stimulate or inhibit) the selectin's function comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In some embodiments, a method for inhibiting the development of a condition associated with an excessive selectin-mediated function comprising administering to a subject in need thereof an effective amount of at least one compound of Formula (I) and/or a pharmaceutical composition comprising at least one compound of Formula (I) and optionally at least one additional pharmaceutically acceptable ingredient is disclosed. In some embodiments, the selectin-mediated function is selectin-mediated intercellular adhesion.

In some embodiments, a method for inhibiting rejection of a transplanted tissue comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In some embodiments, a method for treating sickle cell disease (SCD) or complications associated therewith comprising administering to a subject in need thereof an effective amount of at least one compound of Formula (I) and/or a pharmaceutical composition comprising at least one compound of Formula (I) and optionally at least one additional pharmaceutically acceptable ingredient is disclosed. In some embodiments, this application provides a method for treating vaso-occlusive crisis comprising administering to a subject in need thereof an effective amount of at least one compound of Formula (I) and/or a pharmaceutical composition comprising at least one compound of Formula (I) and optionally at least one additional pharmaceutically acceptable ingredient.

In some embodiments, a method for treating graft versus host disease (GVHD) or complications associated therewith comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In some embodiments, a method for treating

sinusoidal obstruction syndrome (SOS) or complications associated therewith comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In some embodiments, a method for treating cancers of the blood and complications associated therewith comprising administering to a subject in need thereof an effective amount of at least one compound of Formula (I) and/or a pharmaceutical composition comprising at least one compound of Formula (I) and optionally at least one additional pharmaceutically acceptable ingredient is disclosed. Examples of cancers of the blood include, but are not limited to, acute myelogenous leukemia (AML), acute lymphoblastic leukemia (ALL), chronic myelogenous leukemia (CML) and multiple myeloma (MM).

In some embodiments, a method for treating epilepsy comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the prophetic synthesis of building block 11.

FIG. 2 is a diagram illustrating the prophetic synthesis of compound 17.

FIG. 3 is a diagram illustrating the prophetic synthesis of compound 24.

FIG. 4 is a diagram illustrating the prophetic synthesis of compound 32.

FIG. 5 is a diagram illustrating the prophetic synthesis of building block 42.

FIG. 6 is a diagram illustrating the prophetic synthesis of compound 46.

FIG. 7 is a diagram illustrating the prophetic synthesis of compound 51.

FIG. 8 is a diagram illustrating the prophetic synthesis of building block 53.

FIG. 9 is a diagram illustrating the prophetic synthesis of building block 57.

FIG. 10 is a diagram illustrating the prophetic synthesis of compound 60.

Compounds, compositions, and methods for modulating selectin-mediated processes are disclosed. The compounds have a variety of uses in vitro and in vivo.

In some embodiments, disclosed are multimeric selectin-modulators of Formula (I):

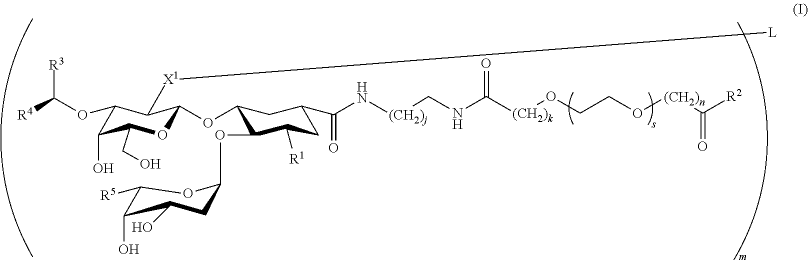

prodrugs thereof, and pharmaceutically acceptable salts of any of the foregoing, wherein

each R¹, which may be identical or different, is independently chosen from H, C₁₋₈ alkyl, C₂₋₈ alkenyl, C₂₋₈ alkynyl, C₄₋₁₆ cycloalkylalkyl,

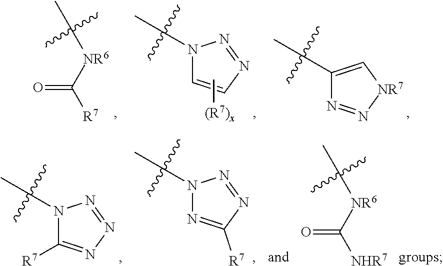

each R², which may be identical or different, is independently chosen from BASA and BACA groups;

each R³, which may be identical or different, is independently chosen from —CN, —CH₂CN, and —C(═O)Y¹ groups, wherein each Y¹, which may be identical or different, is independently chosen from C₁₋₈ alkyl, C₂₋₈ alkenyl, C₂₋₈ alkynyl, —OZ¹, —NHOH, —NHOCH₃, —NHCN, and —NZ¹Z² groups, wherein each Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, wherein Z¹ and Z² may join together along with the nitrogen atom to which they are attached to form a ring;

each R⁴, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₈ aryl groups;

each R⁵, which may be identical or different, is independently chosen from —CN, C₁₋₈ alkyl, and C₁₋₄ haloalkyl groups;

each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups;

each R⁷, which may be identical or different, is independently chosen from H, C₁₋₈ alkyl, C₂₋₈ alkenyl, C₂₋₈ alkynyl, C₄₋₁₆ cycloalkylalkyl,

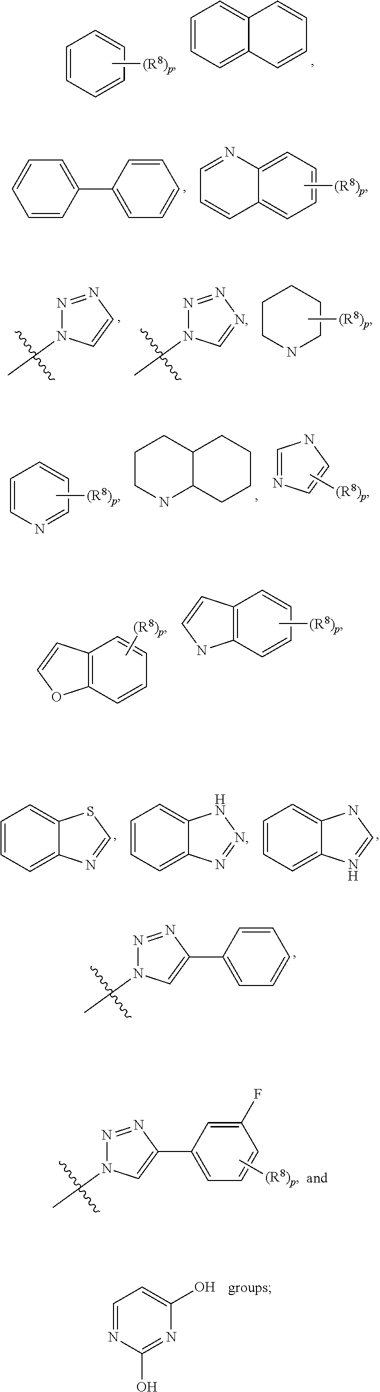

each R⁸, which may be identical or different, is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups;

each X¹, which may be identical or different, is independently chosen from —O— and —N(Z³)—, wherein each Z³, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;

each j, which may be identical or different, is independently chosen from integers ranging from 1 to 29;

each k, which may be identical or different, is independently chosen from integers ranging from 1 to 10;

each n, which may be identical or different, is independently chosen from integers ranging from 1 to 10.

each p, which may be identical or different, is independently chosen from integers ranging from 0 to 3

each s, which may be identical or different, is independently chosen from integers ranging from 1 to 256;

each x, which may be identical or different, is independently chosen from integers ranging from 0 to 2;

m is chosen from integers ranging from 2 to 256; and

L is chosen from linker groups.

As used herein, BASAs (Benzyl Amino Sulfonic Acids) are low molecular weight sulfated compounds which have the ability to interact with a selectin. The interaction modulates or assists in the modulation (e.g., inhibition or enhancement) of a selectin-mediated function (e.g., an intercellular interaction). They exist as either their protonated acid form, or as a sodium salt, although sodium may be replaced with potassium or any other pharmaceutically acceptable counterion.

Portions of BASA that retain the ability to interact with a selectin (which interaction modulates or assists in the modulation of a selectin-mediated function as described herein) are also a “BASA” of the disclosed selectin modulators. Such portions generally comprise at least one aromatic ring present within the BASA structure. In some embodiments, a portion may comprise a single aromatic ring, multiple such rings, or half of a symmetrical BASA molecule.

Analogues of BASA and portions thereof (both of which possess the biological characteristic set forth herein) are also encompassed, e.g., by the “BASA” group of the selectin modulators within the disclosure. As used herein, an “analogue” is a compound that differs from BASA or a portion thereof because of one or more additions, deletions and/or substitutions of chemical moieties, such that the ability of the analogue to inhibit a selectin-mediated interaction is not diminished. For example, an analogue may contain S to P substitutions (e.g., a sulfate group replaced with a phosphate group). Other possible modifications include: (a) modifications to ring size (e.g., any ring may contain between 4 and 7 carbon atoms); (b) variations in the number of fused rings (e.g., a single ring may be replaced with a polycyclic moiety containing up to three fused rings, a polycyclic moiety may be replaced with a single unfused ring or the number of fused rings within a polycyclic moiety may be altered); (c) ring substitutions in which hydrogen atoms or other moieties covalently bonded to a carbon atom within an aromatic ring may be replaced with any of a variety of moieties, such as F, Cl, Br, I, OH, OC₁₋₈ alkyl, SH, NO₂, CN, NH₂, NHC₁₋₈ alkyl, N(C₁₋₈ alkyl)₂, SO₃T (wherein T is chosen from H⁺, Na⁺, K⁺, and other pharmaceutically acceptable counterions), —CO₂T, —PO₄T₂, —SO₂NH₂, C₁₋₈ alkyl, C₆₋₁₀ aryl, —C(═O)OC₁₋₈ alkyl, —CF₂W (wherein W is chosen from H, F, alkyl, aryl, and acyl groups) and carbohydrates; and (d) modifications to linking moieties (i.e., moieties located between rings in the BASA molecule) in which groups such as alkyl, ester, amide, anhydride, and carbamate groups may be substituted for one another.

As used herein, BACAs (Benzyl Amino Carboxylic Acids) is similar to BASAs, except instead of sulfonic acid groups, the compounds possess carboxylic acid groups. For example, the sulfonic acid groups of the above BASA compounds may be replaced with carboxylic acid groups. Thus, the above disclosure to BASAs is incorporated by reference into this description of BACAs.

In some embodiments, each R¹, which may be identical or different, is independently chosen from H, C₁₋₈ alkyl groups, C₂₋₈ alkenyl, and C₂₋₈ alkynyl groups. In some embodiments, each R¹, which may be identical or different, is independently chosen from H and C₁₋₈ alkyl groups. In some embodiments, each R¹, which may be identical or different, is independently chosen from C₁₋₈ alkyl groups. In some embodiments, each R¹, which may be identical or different, is independently chosen from C₁₋₄ alkyl groups.

In some embodiments, each R¹, which may be identical or different, is independently chosen from

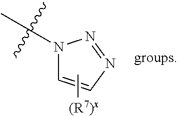

In some embodiments, each R¹, which may be identical or different, is independently chosen from

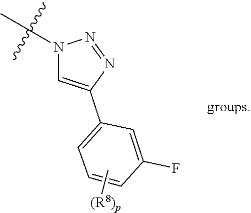

In some embodiments, each R¹, which may be identical or different, is independently chosen from

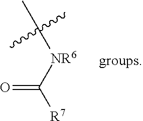

In some embodiments, at least one R¹ is chosen from H, C₁₋₈ alkyl groups, C₂₋₈ alkenyl, and C₂₋₈ alkynyl groups. In some embodiments, at least one R¹ is chosen from H and C₁₋₈ alkyl groups. In some embodiments, at least one R¹ is H. In some embodiments, at least one R¹ is chosen from C₁₋₈ alkyl groups. In some embodiments, at least one R¹ is chosen from C₁₋₄ alkyl groups. In some embodiments, at least one R¹ is methyl. In some embodiments, at least one R¹ is ethyl.

In some embodiments, at least one R¹ is chosen from

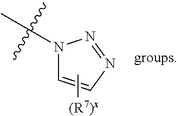

In some embodiments, at least one R¹ is chosen from

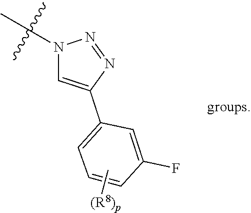

In some embodiments, at least one R¹ is

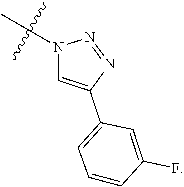 [SMILES: *n1cc(-c2cccc(F)c2)nn1]

In some embodiments, at least one R¹ is chosen from

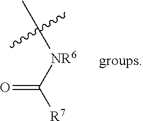

In some embodiments, at least one R¹ is

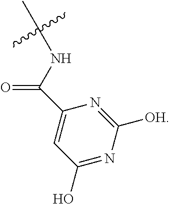 [SMILES: *NC(=O)c1cc(O)nc(O)n1]

In some embodiments, each R¹ is identical and chosen from H, C₁₋₈ alkyl groups, C₂₋₈ alkenyl, and C₂₋₈ alkynyl groups. In some embodiments, each R¹ is identical and chosen from H and C₁₋₈ alkyl groups. In some embodiments, each R¹ is H. In some embodiments, each R¹ is identical and chosen from C₁₋₈ alkyl groups. In some embodiments, each R¹ is identical and chosen from C1-4 alkyl groups. In some embodiments, each R¹ is methyl. In some embodiments, each R¹ is ethyl.

In some embodiments, each R¹ is identical and chosen from

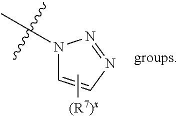

In some embodiments, each R¹ is identical and chosen from

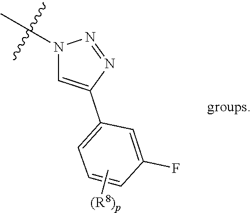

In some embodiments, each R¹ is identical and chosen from

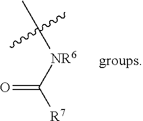

In some embodiments, each R¹ is

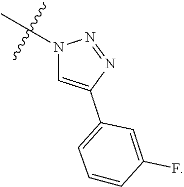 [SMILES: *n1cc(-c2cccc(F)c2)nn1]

In some embodiments, each R¹ is

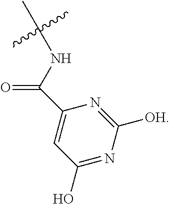 [SMILES: *NC(=O)c1cc(O)nc(O)n1]

In some embodiments, each R², which may be identical or different, is independently chosen from BASA groups.

In some embodiments, at least one R² is chosen from BASA groups.

In some embodiments, each R² is identical and chosen from BASA groups.

In some embodiments, each R², which may be identical or different, comprises a compound independently chosen from

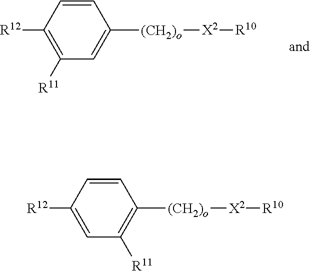

wherein

each R¹⁰, which may be identical or different, is independently chosen from H, —F, and —C(═O)OY³ groups;

each R¹¹, which may be identical or different, is independently chosen from H, —PO₃T₂, —SO₃T₂, —CH₂—PO₃T₂, —CH₂—SO₃T₂, —CF₃, —CHR¹³R¹⁴, —CH₂CHR¹³R¹⁴, —(CH₂)₂CHR¹³R¹⁴, —(CH₂)₃CHR¹³R¹⁴, and —NZ⁴Z⁵ groups, wherein each R¹³ and R¹⁴, which may be identical or different, are independently chosen from H, —C(═O)OY³, and —NH₂ groups, wherein each Z⁴ and Z⁵, which may be identical or different, are independently chosen from H and —Y⁴—(C₆₋₁₈ aryl) groups, wherein each Y⁴, which may be identical or different, is independently chosen from C₁₋₄ alkyl and —C(═O) groups;

each R¹², which may be identical or different, is independently chosen from H, —PO₃T₂, —SO₃T₂, —CH₂—PO₃T₂, —CH₂—SO₃T₂, —CF₃, —CHR¹⁵R¹⁶, —CH₂CHR¹⁵R¹⁶, —(CH₂)₂CHR¹⁵R¹⁶, —(CH₂)₃CHR¹⁵R¹⁶, and —NZ⁶Z⁷ groups, wherein each R¹⁵ and R¹⁶, which may be identical or different, are independently chosen from H, —C(═O)OY³, —NH₂ groups, wherein each Z⁶ and Z⁷, which may be identical or different, are independently chosen from H and —Y⁵—(C₆₋₁₈ aryl) groups, wherein each Y⁵, which may be identical or different, is independently chosen from C₁₋₄ alkyl and —C(═O) groups;

each X², which may be identical or different, is independently chosen from —PO₂T-, —SO₂T-, and —CF₂—;

each Y³, which may be identical or different, is independently chosen from H, C₁₋₄ alkyl, and C₆₋₁₈ aryl groups;

each T, which may be identical or different, is independently chosen from H and pharmaceutically acceptable counterions; and

each o, which may be identical or different, is independently chosen from integers ranging from 0 to 1.

In some embodiments, at least one R² comprises a compound chosen from

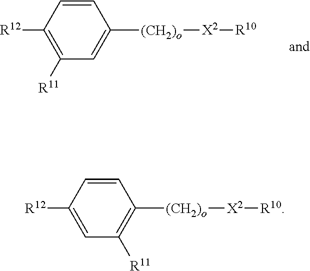

In some embodiments, each R² is identical and comprises a compound chosen from

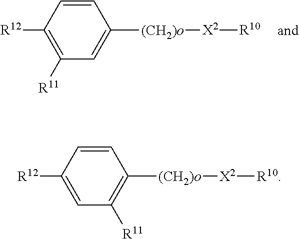

In some embodiments, each R², which may be identical or different, is independently chosen from

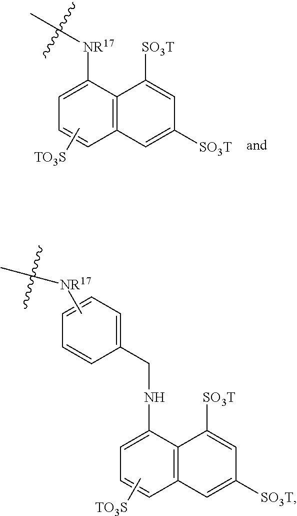

wherein

each R¹⁷, which may be identical or different, is independently chosen from H, C₁₋₈ alkyl, —C(═O)X³, and —C(═O)NHX³ groups, wherein each X³, which may be identical or different, is independently chosen from C₁₋₈ alkyl, C₂₋₈ alkenyl, C₂₋₈ alkynyl, C₆₋₁₈ aryl, and C₁₋₁₃ heteroaryl groups; and

each T, which may be identical or different, is independently chosen from H and pharmaceutically acceptable counterions.

In some embodiments, each R², which may be identical or different, is independently chosen from

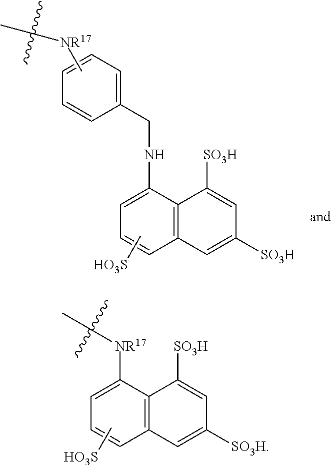

In some embodiments, each R², which may be identical or different, is independently chosen from

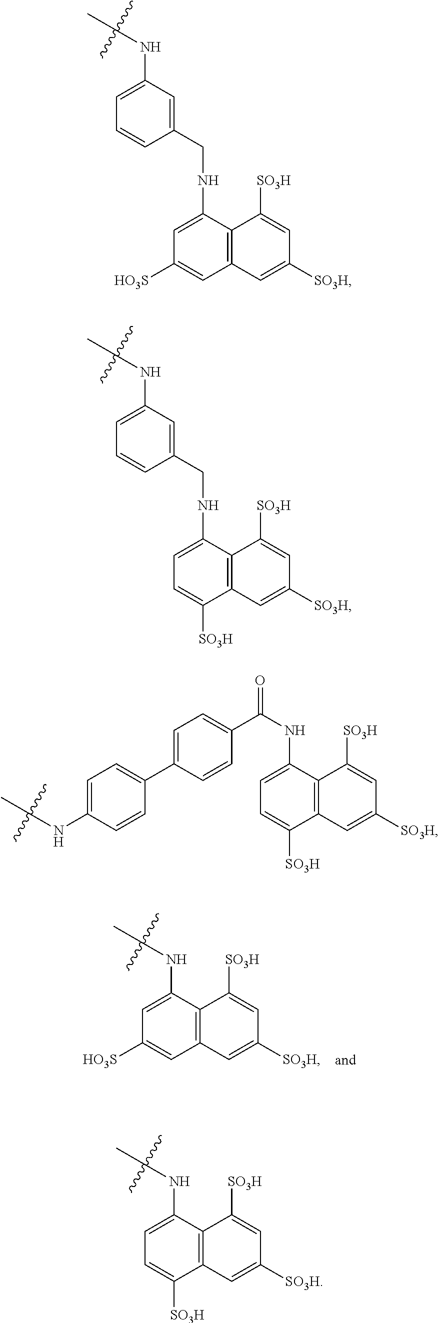 [SMILES: *Nc1cccc(CNc2cc(S(=O)(=O)O)cc3cc(S(=O)(=O)O)cc(S(=O)(=O)O)c23)c1 ; *Nc1cccc(CNc2ccc(S(=O)(=O)O)c3cc(S(=O)(=O)O)cc(S(=O)(=O)O)c23)c1 ; *Nc1ccc(-c2ccc(C(=O)Nc3ccc(S(=O)(=O)O)c4cc(S(=O)(=O)O)cc(S(=O)(=O)O)c34)cc2)cc1 ; *Nc1cc(S(=O)(=O)O)cc2cc(S(=O)(=O)O)cc(S(=O)(=O)O)c12 ; "and" ; *Nc1ccc(S(=O)(=O)O)c2cc(S(=O)(=O)O)cc(S(=O)(=O)O)c12]

In some embodiments, at least one R² is chosen from

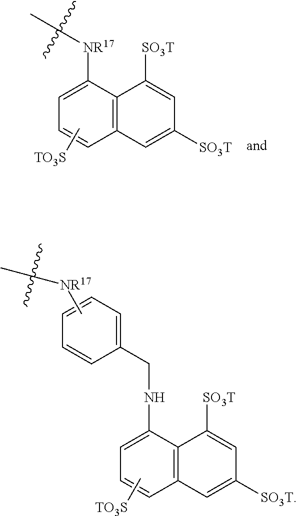

In some embodiments, at least one R² is chosen from

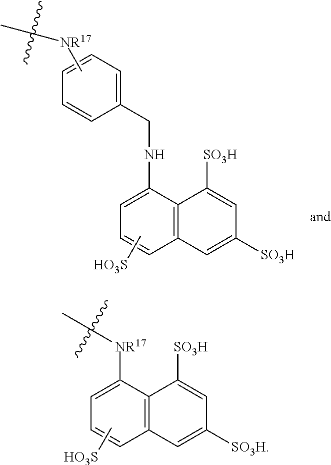

In some embodiments, at least one R² is chosen from

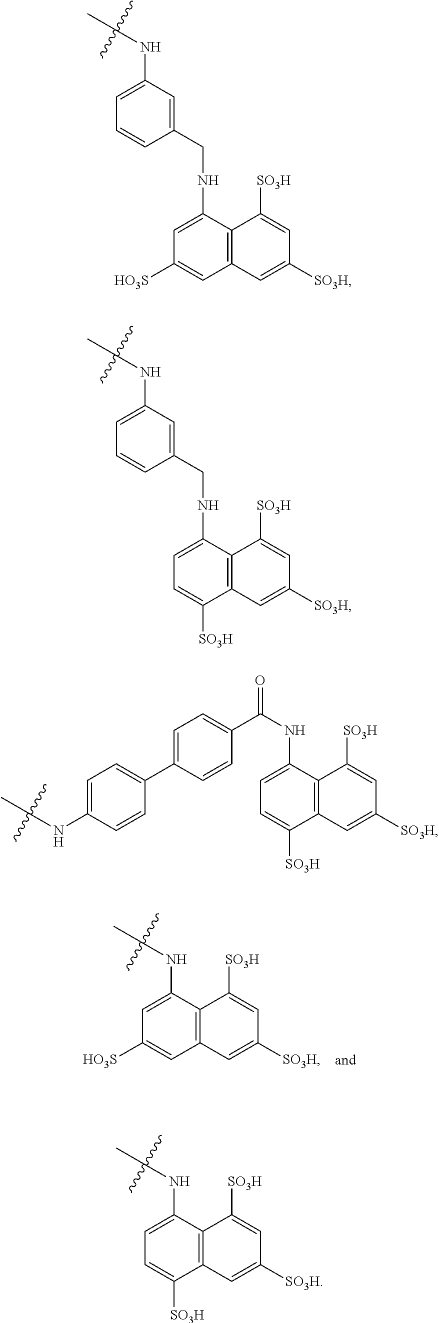 [SMILES: *Nc1cccc(CNc2cc(S(=O)(=O)O)cc3cc(S(=O)(=O)O)cc(S(=O)(=O)O)c23)c1 ; *Nc1cccc(CNc2ccc(S(=O)(=O)O)c3cc(S(=O)(=O)O)cc(S(=O)(=O)O)c23)c1 ; *Nc1ccc(-c2ccc(C(=O)Nc3ccc(S(=O)(=O)O)c4cc(S(=O)(=O)O)cc(S(=O)(=O)O)c34)cc2)cc1 ; *Nc1cc(S(=O)(=O)O)cc2cc(S(=O)(=O)O)cc(S(=O)(=O)O)c12 ; "and" ; *Nc1ccc(S(=O)(=O)O)c2cc(S(=O)(=O)O)cc(S(=O)(=O)O)c12]

In some embodiments, at least one R² is

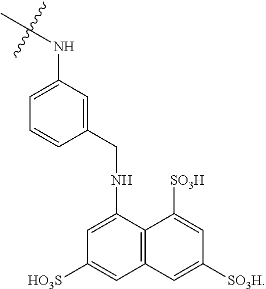 [SMILES: *Nc1cccc(CNc2cc(S(=O)(=O)O)cc3cc(S(=O)(=O)O)cc(S(=O)(=O)O)c23)c1]

In some embodiments, at least one R² is

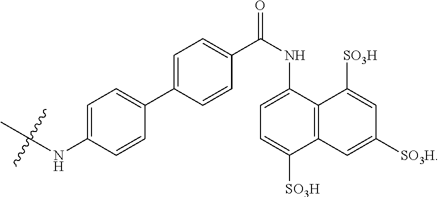 [SMILES: *Nc1ccc(-c2ccc(C(=O)Nc3ccc(S(=O)(=O)O)c4cc(S(=O)(=O)O)cc(S(=O)(=O)O)c34)cc2)cc1]

In some embodiments, at least one R² is chosen from

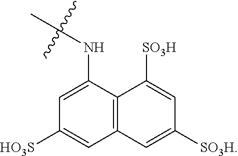 [SMILES: *Nc1cc(S(=O)(=O)O)cc2cc(S(=O)(=O)O)cc(S(=O)(=O)O)c12]

In some embodiments, each R² is identical and chosen from

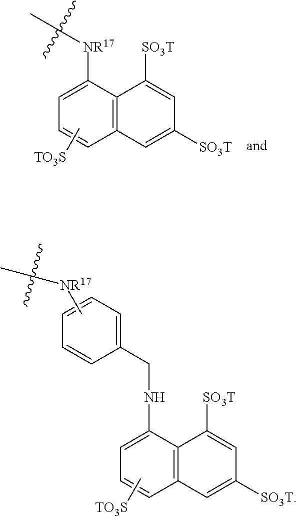

In some embodiments, each R² is identical and chosen from

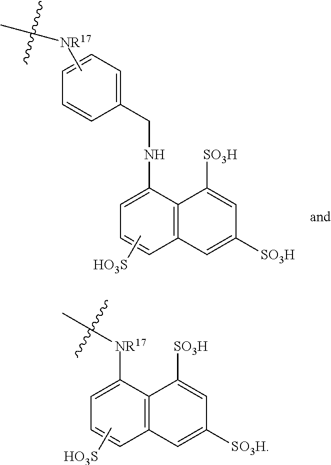

In some embodiments, each R² is identical and chosen from

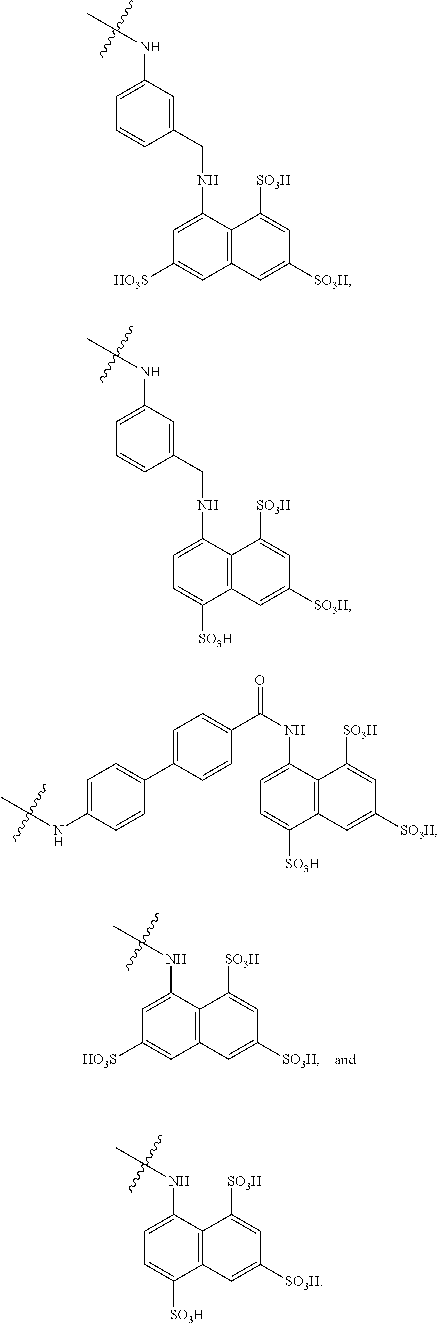 [SMILES: *Nc1cccc(CNc2cc(S(=O)(=O)O)cc3cc(S(=O)(=O)O)cc(S(=O)(=O)O)c23)c1 ; *Nc1cccc(CNc2ccc(S(=O)(=O)O)c3cc(S(=O)(=O)O)cc(S(=O)(=O)O)c23)c1 ; *Nc1ccc(-c2ccc(C(=O)Nc3ccc(S(=O)(=O)O)c4cc(S(=O)(=O)O)cc(S(=O)(=O)O)c34)cc2)cc1 ; *Nc1cc(S(=O)(=O)O)cc2cc(S(=O)(=O)O)cc(S(=O)(=O)O)c12 ; "and" ; *Nc1ccc(S(=O)(=O)O)c2cc(S(=O)(=O)O)cc(S(=O)(=O)O)c12]

In some embodiments, each R² is

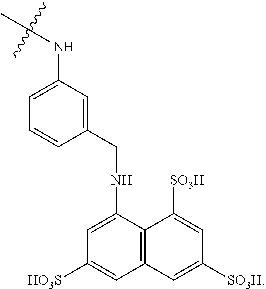 [SMILES: *Nc1cccc(CNc2cc(S(=O)(=O)O)cc3cc(S(=O)(=O)O)cc(S(=O)(=O)O)c23)c1]

In some embodiments, each R² is

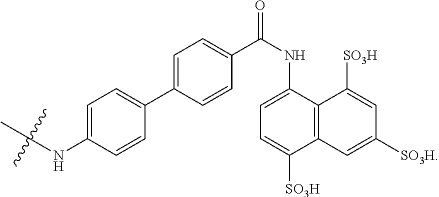 [SMILES: *Nc1ccc(-c2ccc(C(=O)Nc3ccc(S(=O)(=O)O)c4cc(S(=O)(=O)O)cc(S(=O)(=O)O)c34)cc2)cc1]

In some embodiments, each R² is

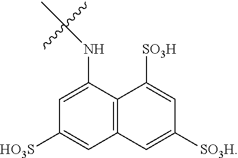 [SMILES: *Nc1cc(S(=O)(=O)O)cc2cc(S(=O)(=O)O)cc(S(=O)(=O)O)c12]

In some embodiments, each R², which may be identical or different, is independently chosen from BACA groups.

In some embodiments, at least one R² is chosen from BACA groups.

In some embodiments, each R² is identical and chosen from BACA groups.

In some embodiments, each R², which may be identical or different, comprises a compound independently chosen from

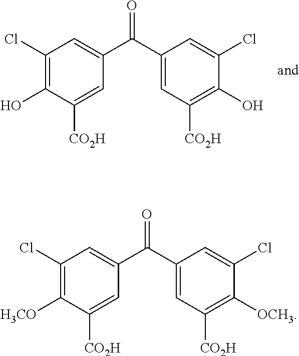 [SMILES: O=C(c1cc(Cl)c(O)c(C(=O)O)c1)c1cc(Cl)c(O)c(C(=O)O)c1 ; COc1c(Cl)cc(C(=O)c2cc(Cl)c(OC)c(C(=O)O)c2)cc1C(=O)O]

In some embodiments, at least one R² comprises a compound chosen from

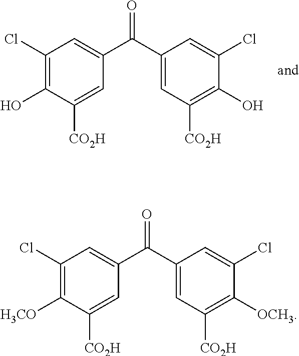 [SMILES: O=C(c1cc(Cl)c(O)c(C(=O)O)c1)c1cc(Cl)c(O)c(C(=O)O)c1 ; COc1c(Cl)cc(C(=O)c2cc(Cl)c(OC)c(C(=O)O)c2)cc1C(=O)O]

In some embodiments, each R² is identical and comprises a compound chosen from

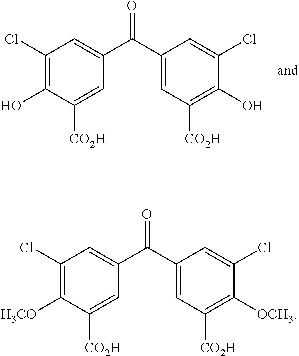 [SMILES: O=C(c1cc(Cl)c(O)c(C(=O)O)c1)c1cc(Cl)c(O)c(C(=O)O)c1 ; COc1c(Cl)cc(C(=O)c2cc(Cl)c(OC)c(C(=O)O)c2)cc1C(=O)O]

In some embodiments, each R², which may be identical or different, is independently chosen from

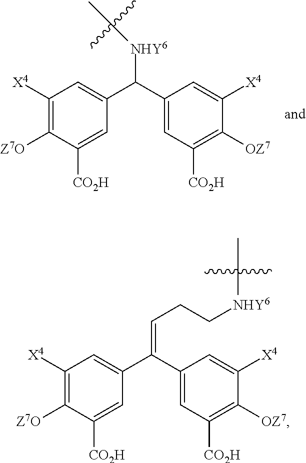

wherein
each X⁴, which may be identical or different, is independently chosen from F and Cl;
each Y⁶, which may be identical or different, is independently chosen from H, —C(═O)(OCH₂CH₂)ᵤ and —C(═O)(CH₂)ᵤ groups, wherein each u, which may be identical or different, is independently chosen from integers ranging from 0 to 8; and
each Z⁷, which may be identical or different, is independently chosen from H, C₁₋₈ alkyl, C₂₋₈ alkenyl, and C₂₋₈ alkynyl groups.

In some embodiments, at least one R² is chosen from

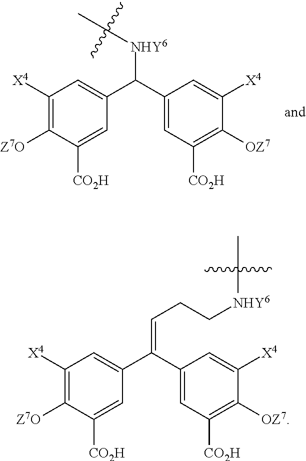

In some embodiments, each R² is identical and chosen from

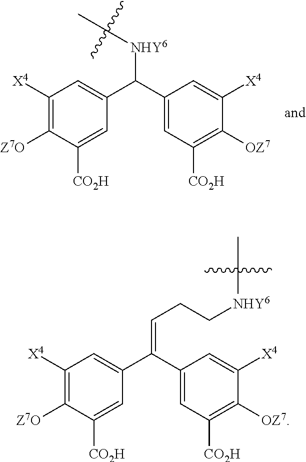

In some embodiments, each R³, which may be identical or different, is PG independently chosen from —C(═O)Y¹ groups, wherein each Y¹, which may be identical or different, is independently chosen from —OZ¹, —NHOH, —NHOCH₃, and —NZ¹Z² groups, wherein each of Z¹ and Z², which may be identical or different, are independently chosen from H, C₁₋₈ alkyl, C₁₋₈ haloalkyl, and C₇₋₁₂ arylalkyl groups.

In some embodiments, each R³, which may be identical or different, is independently chosen from —C(═O)OZ¹ groups, wherein each Z¹, which may be identical or different, is independently chosen from H, C₁₋₈ alkyl, C₁₋₈ haloalkyl, and C₇₋₁₂ arylalkyl groups.

In some embodiments, each R³, which may be identical or different, is independently chosen from —C(═O)NZ¹Z² groups, wherein each of Z¹ and Z², which may be identical or different, are independently chosen from H, C₁₋₈ alkyl, C₁₋₈ haloalkyl, and C₇₋₁₂ arylalkyl groups, wherein Z¹ and Z² may join together along with the nitrogen atom to which they are attached to form a ring.

In some embodiments, each of Z¹ and Z², which may be identical or different, are independently chosen from H, C₁₋₈ alkyl, C₁₋₈ haloalkyl, and C₇₋₁₂ arylalkyl groups. In some embodiments, at least one of Z¹ and Z² is H. In some embodiments, each of Z¹ and Z² is H. In some embodiments, at least one of Z¹ and Z² is methyl. In some embodiments, each of Z¹ and Z² is methyl. In some embodiments, at least one of Z¹ and Z² is ethyl. In some embodiments, each of Z¹ and Z² is ethyl. In some embodiments, Z¹ is H and Z² is methyl. In some embodiments, Z¹ and Z² join together along with the nitrogen atom to which they are attached to form a ring.

In some embodiments, each R³, which may be identical or different, is independently chosen from

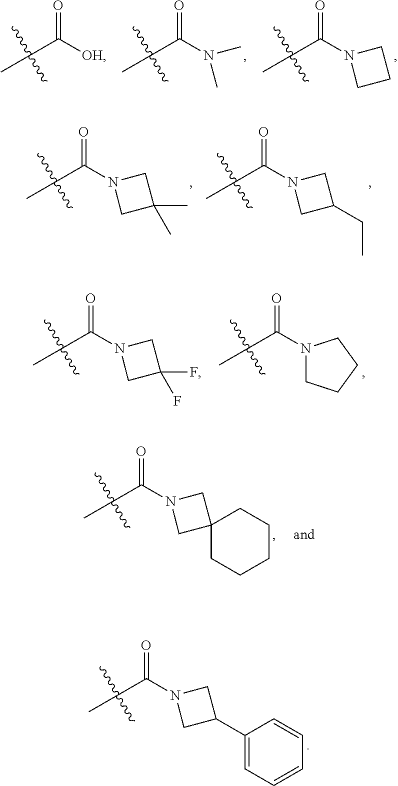

In some embodiments, each R³, which may be identical or different, is independently chosen from

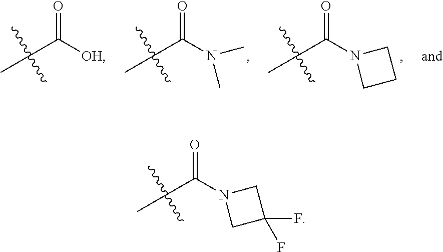

In some embodiments, at least one R³ is chosen from —C(═O)Y¹ groups. In some embodiments, at least one R³ is chosen from —C(═O)OZ¹ groups. In some embodiments, at least one R³ is chosen from —C(═O)NZ¹Z² groups.

In some embodiments, at least one R³ is chosen from

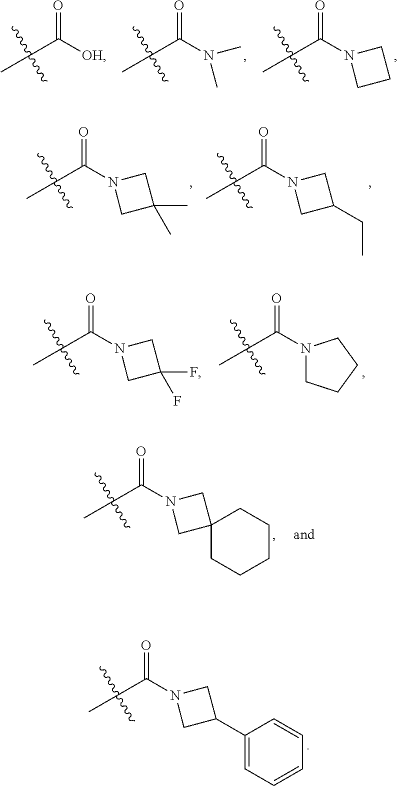

In some embodiments, at least one R³ is chosen from

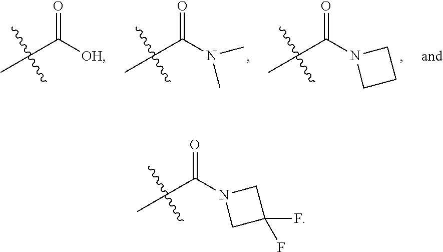

In some embodiments, each R³ is identical and chosen from —C(═O)Y¹ groups. In some embodiments, each R³ is identical and chosen from —C(═O)OZ¹ groups. In some embodiments, each R³ is identical and chosen from —C(═O)NZ¹Z² groups.

In some embodiments, each R³ is identical and chosen from

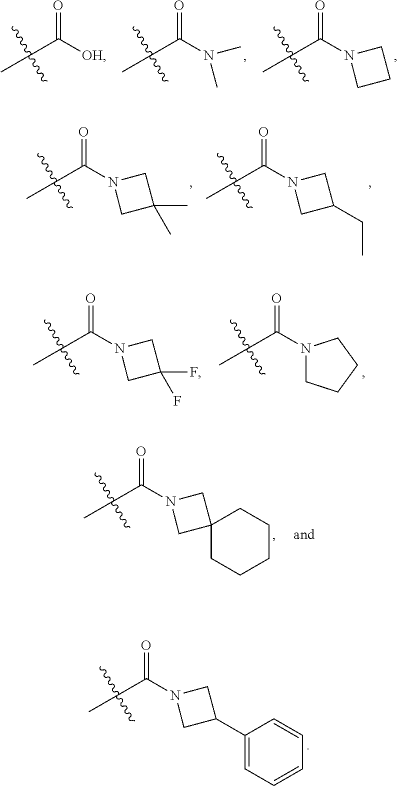

In some embodiments, each R³ is identical and chosen from

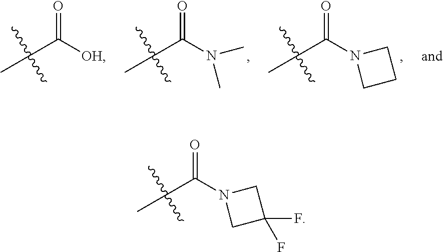

In some embodiments, each R³ is

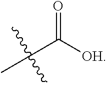 [SMILES: *C(=O)O]

In some embodiments, each R³ is

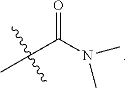 [SMILES: *C(=O)N(C)C]

In some embodiments, each R³ is

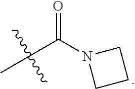 [SMILES: *C(=O)N1CCC1]

In some embodiments, each R³ is

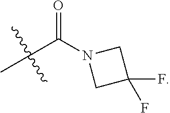 [SMILES: *C(=O)N1CC(F)(F)C1]

In some embodiments, each R⁴, which may be identical or different, is independently chosen from C₁₋₈ alkyl groups. In some embodiments, each R⁴, which may be identical or different, is independently chosen from C₁₋₈ haloalkyl groups. In some embodiments, each R⁴, which may be identical or different, is independently chosen from C₄₋₁₆ cycloalkylalkyl groups. In some embodiments, each R⁴, which may be identical or different, is independently chosen from C₄₋₈ cycloalkylalkyl groups. In some embodiments, each R⁴, which may be identical or different, is independently chosen from propyl, cyclopropylmethyl, and cyclohexylmethyl.

In some embodiments, at least one R⁴ is chosen from C₁₋₈ alkyl groups. In some embodiments, at least one R⁴ is chosen from C₁₋₈ haloalkyl groups. In some embodiments, at least one R⁴ is chosen from C₄₋₁₆ cycloalkylalkyl groups. In some embodiments, at least one R⁴ is chosen from C₄₋₈ cycloalkylalkyl groups. In some embodiments, at least one R⁴ is chosen from propyl, cyclopropylmethyl, and cyclohexylmethyl. In some embodiments, at least one R⁴ is propyl. In some embodiments, at least one R⁴ is cyclopropylmethyl. In some embodiments, at least one R⁴ is cyclohexylmethyl.

In some embodiments, each R⁴ is identical and chosen from C₁₋₈ alkyl groups. In some embodiments, each R⁴ is identical and chosen from C₁₋₈ haloalkyl groups. In some embodiments, each R⁴ is identical and chosen from C₄₋₁₆ cycloalkylalkyl groups. In some embodiments, each R⁴ is identical and chosen from C₄₋₈ cycloalkylalkyl groups. In some embodiments, each R⁴ is identical and chosen from propyl, cyclopropylmethyl, and cyclohexylmethyl. In some embodiments, each R⁴ is propyl. In some embodiments, each R⁴ is cyclopropylmethyl. In some embodiments, each R⁴ is cyclohexylmethyl.

In some embodiments, each R⁵, which may be identical or different, is independently chosen from C₁₋₈ alkyl groups. In some embodiments, each R⁵, which may be identical or different, is independently chosen from C₁₋₄ alkyl groups. In some embodiments, each R⁵, which may be identical or different, is independently chosen from C₁₋₄ haloalkyl groups. In some embodiments, each R⁵, which may be identical or different, is independently chosen from halomethyl groups. In some embodiments, each R⁵, which may be identical or different, is independently chosen from CF₃, CH₃, and CN.

In some embodiments, at least one R⁵ is chosen from C₁₋₈ alkyl groups. In some embodiments, at least one R⁵ is chosen from C₁₋₄ alkyl groups. In some embodiments, at least one R⁵ is chosen from C₁₋₄ haloalkyl groups. In some embodiments, at least one R⁵ is chosen from halomethyl groups. In some embodiments, at least one R⁵ is independently chosen from CF₃, CH₃, and CN. In some embodiments, at least one R⁵ is CF₃. In some embodiments, at least one R⁵ is CH₃. In some embodiments, at least one R⁵ is CN.

In some embodiments, each R⁵ is identical and chosen from C₁₋₈ alkyl groups. In some embodiments, each R⁵ is identical and chosen from C₁₋₄ alkyl groups. In some embodiments, each R⁵ is identical and chosen from C₁₋₄ haloalkyl groups. In some embodiments, each R⁵ is identical and chosen from halomethyl groups. In some embodiments, each R⁵ is identical and chosen from CF₃, CH₃, and CN. In some embodiments, each R⁵ is CF₃. In some embodiments, each R⁵ is CH₃. In some embodiments, each R⁵ is CN.

In some embodiments, each R⁶, which may be identical or different, is independently chosen from H and C₁₋₈ alkyl groups. In some embodiments, each R⁶, which may be identical or different, is independently chosen from C₁₋₈ alkyl groups. In some embodiments, each R⁶, which may be identical or different, is independently chosen from C₁₋₄ alkyl groups. In some embodiments, each R⁶, which may be identical or different, is independently chosen from methyl and ethyl. In some embodiments, each R⁶, which may be identical or different, is independently chosen from —C(═O)R⁷ groups.

In some embodiments, at least one R⁶ is chosen from H and C₁₋₈ alkyl groups. In some embodiments, at least one R⁶ is H. In some embodiments, at least one R⁶ is chosen from C₁₋₈ alkyl groups. In some embodiments, at least one R⁶ is chosen from C₁₋₄ alkyl groups. In some embodiments, at least one R⁶ is chosen from methyl and ethyl. In some embodiments, at least one R⁶ is chosen from —C(═O)R⁷ groups.

In some embodiments, each R⁶ is identical and chosen from H and C₁₋₈ alkyl groups. In some embodiments, each R⁶ is H. In some embodiments, each R⁶ is identical and chosen from C₁₋₈ alkyl groups. In some embodiments, each R⁶ is identical and chosen from C₁₋₄ alkyl groups. In some embodiments, each R⁶ is identical and chosen from methyl and ethyl. In some embodiments, each R⁶ is identical and chosen from —C(═O)R⁷ groups.

In some embodiments, each R⁷, which may be identical or different, is independently chosen from H, C₁₋₈ alkyl, and C₄₋₁₆ cycloalkylalkyl groups. In some embodiments, each R⁷, which may be identical or different, is independently chosen from H and C₁₋₈ alkyl groups. In some embodiments, each R⁷, which may be identical or different, is independently chosen from C₁₋₄ alkyl groups. In some embodiments, each R⁷, which may be identical or different, is independently chosen from propyl groups. In some embodiments, each R⁷, which may be identical or different, is independently chosen from methyl and ethyl.

In some embodiments, each R⁷, which may be identical or different, is independently chosen from

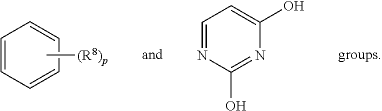

In some embodiments, each R⁷, which may be identical or different, is independently chosen from

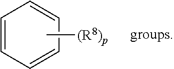

In some embodiments, at least one R⁷ is chosen from H, C₁₋₈ alkyl, and C₄₋₁₆ cycloalkylalkyl groups. In some embodiments, at least one R⁷ is chosen from H and C₁₋₈ alkyl groups. In some embodiments, at least one R⁷ is H. In some embodiments, at least one R₇ is chosen from C₁₋₄ alkyl groups. In some embodiments, at least one R⁷ is chosen from propyl groups. In some embodiments, at least one R⁷ is ethyl. In some embodiments, at least one R⁷ is methyl.

In some embodiments, at least one R⁷ is chosen from

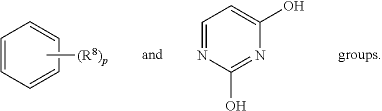

In some embodiments, at least one R⁷ is chosen from

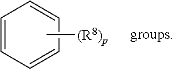

In some embodiments, at least one R⁷ is

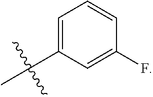 [SMILES: *c1cccc(F)c1]

In some embodiments, at least one R⁷ is

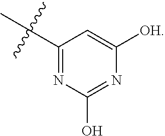 [SMILES: *c1cc(O)nc(O)n1]

In some embodiments, each R⁷ is identical and chosen from H, C₁₋₈ alkyl, and C₄₋₁₆ cycloalkylalkyl groups. In some embodiments, each R⁷ is identical and chosen from H and C₁₋₈ alkyl groups. In some embodiments, each R⁷ is H. In some embodiments, each R⁷ is identical and chosen from C₁₋₄ alkyl groups. In some embodiments, each R⁷ is identical and chosen from propyl groups. In some embodiments, each R⁷ is ethyl. In some embodiments, each R⁷ is methyl.

In some embodiments, each R⁷ is identical and chosen from

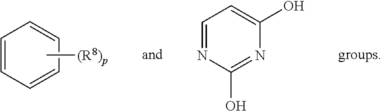

In some embodiments, each R⁷ is identical and chosen from

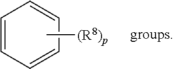

In some embodiments, each R⁷ is

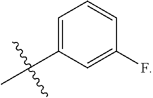 [SMILES: *c1cccc(F)c1]

In some embodiments, each R⁷ is

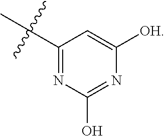 [SMILES: *c1cc(O)nc(O)n1]

In some embodiments, each R⁸, which may be identical or different, is independently chosen from H, —OH, Cl, F, N₃, —NH₂, C₁₋₈ alkyl, C₆₋₁₄ aryl, —OC₁₋₈ alkyl, and —OC₆₋₁₄ aryl groups. In some embodiments, each R⁸, which may be identical or different, is independently chosen from H, —OH, Cl, F, N₃, —NH₂, C₁₋₈ alkyl, and —OC₁₋₈ alkyl groups. In some embodiments, each R⁸, which may be identical or different, is independently chosen from H,

—OH, Cl, F, N₃, —NH₂, C₁₋₄alkyl, and —OC₁₋₄ alkyl groups. In some embodiments, each R⁸, which may be identical or different, is independently chosen from H, —OH, Cl, F, N₃, —NH₂, —CH₃, —CH₂CH₃, —OCH₃, and —OCH₂CH₃.

In some embodiments, at least one Re is chosen from H, —OH, Cl, F, N₃, —NH₂, C₁₋₈ alkyl, C₆₋₁₄ aryl, —OC₁₋₈ alkyl, and —OC₆₋₁₄ aryl groups. In some embodiments, at least one R⁸ is chosen from H, —OH, Cl, F, N₃, —NH₂, C₁₋₈ alkyl, and —OC₁₋₈ alkyl groups. In some embodiments, at least one R⁸ is chosen from H, —OH, Cl, F, N₃, —NH₂, C₁₋₄ alkyl, and —OC₁₋₄ alkyl groups. In some embodiments, at least one R⁸ is chosen from H, —OH, Cl, F, N₃, —NH₂, —CH₃, —CH₂CH₃, —OCH₃, and —OCH₂CH₃. In some embodiments, at least one R⁸ is H.

In some embodiments, each R⁸ is identical and chosen from H, —OH, Cl, F, N₃, —NH₂, C₁₋₈ alkyl, C₆₋₁₄ aryl, —OC₁₋₈ alkyl, and —OC₆₋₁₄ aryl groups. In some embodiments, each R⁸ is identical and chosen from H, —OH, Cl, F, N₃, —NH₂, C₁₋₈ alkyl, and —OC₁₋₈ alkyl groups. In some embodiments, each R⁸ is identical and chosen from H, —OH, Cl, F, N₃, —NH₂, C₁₋₄ alkyl, and —OC₁₋₄ alkyl groups. In some embodiments, each R⁸ is identical and chosen from H, —OH, Cl, F, N₃, —NH₂, —CH₃, —CH₂CH₃, —OCH₃, and —OCH₂CH₃. In some embodiments, each R⁸ is H.

In some embodiments, each X¹, which may be identical or different, is independently chosen from —N(Z³)— groups, wherein each Z³, which may be identical or different, is independently chosen from H, C₁₋₈ alkyl, and C₁₋₈ haloalkyl groups. In some embodiments, each Z³, which may be identical or different, is independently chosen from H and C₁₋₄ alkyl groups.

In some embodiments, at least one X¹ is —O—. In some embodiments, at least one X¹ is chosen from —N(Z³)— groups. In some embodiments, at least one Z³ is chosen from H and C₁₋₄ alkyl groups. In some embodiments, at least one X¹ is —NH—.

In some embodiments, each X¹ is —O—. In some embodiments, each X¹ is identical and chosen from —N(Z³)— groups. In some embodiments, each X¹ is —NH—.

In some embodiments, each j, which may be identical or different, is independently chosen from integers ranging from 1 to 20. In some embodiments, each j, which may be identical or different, is independently chosen from integers ranging from 1 to 10. In some embodiments, each j, which may be identical or different, is independently chosen from integers ranging from 1 to 5.

In some embodiments, at least one j is chosen from integers ranging from 1 to 20. In some embodiments, at least one j is chosen from integers ranging from 1 to 10. In some embodiments, at least one j is chosen from integers ranging from 1 to 5. In some embodiments, at least one j is 5. In some embodiments, at least one j is 4. In some embodiments, at least one j is 3. In some embodiments, at least one j is 2. In some embodiments, at least one j is 1.

In some embodiments, each j is identical and chosen from integers ranging from 1 to 20. In some embodiments, each j is identical and chosen from integers ranging from 1 to 10. In some embodiments, each j is identical and chosen from integers ranging from 1 to 5. In some embodiments, each j is 5. In some embodiments, each j is 4. In some embodiments, each j is 3. In some embodiments, each j is 2. In some embodiments, each j is 1.

In some embodiments, each k, which may be identical or different, is independently chosen from integers ranging from 1 to 7. In some embodiments, each k, which may be identical or different, is independently chosen from integers ranging from 1 to 5. In some embodiments, each k, which may be identical or different, is independently chosen from integers ranging from 1 to 3.

In some embodiments, at least one k is chosen from integers ranging from 1 to 7. In some embodiments, at least one k is chosen from integers ranging from 1 to 5. In some embodiments, at least one k is chosen from integers ranging from 1 to 3. In some embodiments, at least one k is 3. In some embodiments, at least one k is 2. In some embodiments, at least one k is 1.

In some embodiments, each k is identical and chosen from integers ranging from 1 to 7. In some embodiments, each k is identical and chosen from integers ranging from 1 to 5. In some embodiments, each k is identical and chosen from integers ranging from 1 to 3. In some embodiments, each k is 3. In some embodiments, each k is 2. In some embodiments, each k is 1.

In some embodiments, each n, which may be identical or different, is independently chosen from integers ranging from 1 to 7. In some embodiments, each n, which may be identical or different, is independently chosen from integers ranging from 1 to 5. In some embodiments, each n, which may be identical or different, is independently chosen from integers ranging from 1 to 3.

In some embodiments, at least one n is chosen from integers ranging from 1 to 7. In some embodiments, at least one n is chosen from integers ranging from 1 to 5. In some embodiments, at least one n is chosen from integers ranging from 1 to 3. In some embodiments, at least one n is 3. In some embodiments, at least one n is 2. In some embodiments, at least one n is 1.

In some embodiments, each n is identical and chosen from integers ranging from 1 to 7. In some embodiments, each n is identical and chosen from integers ranging from 1 to 5. In some embodiments, each n is identical and chosen from integers ranging from 1 to 3. In some embodiments, each n is 3. In some embodiments, each n is 2. In some embodiments, each n is 1.

In some embodiments, at least one p is 3. In some embodiments, at least one p is 2. In some embodiments, at least one p is 1. In some embodiments, at least one p is 0. In some embodiments, each p is 3. In some embodiments, each p is 2. In some embodiments, each p is 1. In some embodiments, each p is 0.

In some embodiments, each s, which may be identical or different, is independently chosen from integers ranging from 0 to 128. In some embodiments, each s, which may be identical or different, is independently chosen from integers ranging from 1 to 64. In some embodiments, each s, which may be identical or different, is independently chosen from integers ranging from 1 to 32. In some embodiments, each s, which may be identical or different, is independently chosen from integers ranging from 1 to 16. In some embodiments, each s, which may be identical or different, is independently chosen from integers ranging from 1 to 8. In some embodiments, each s, which may be identical or different, is independently chosen from integers ranging from 1 to 4.

In some embodiments, at least one s is chosen from integers ranging from 0 to 128. In some embodiments, at least one s is chosen from integers ranging from 1 to 64. In some embodiments, at least one s is chosen from integers ranging from 1 to 32. In some embodiments, at least one s is chosen from integers ranging from 1 to 16. In some embodiments, at least one s is chosen from integers ranging from 1 to 8. In some embodiments, at least one s is chosen from integers ranging from 1 to 4. In some embodiments, at least one s is 4. In some embodiments, at least one s is 3. In some embodiments, at least one s is 2. In some embodiments, at least one s is 1. In some embodiments, at least one s is 0.

In some embodiments, each s is identical and chosen from integers ranging from 0 to 128. In some embodiments, each s is identical and chosen from integers ranging from 1 to 64. In some embodiments, each s is identical and chosen from integers ranging from 1 to 32. In some embodiments, each s is identical and chosen from integers ranging from 1 to 16. In some embodiments, each s is identical and chosen from integers ranging from 1 to 8. In some embodiments, each s is identical and chosen from integers ranging from 1 to 4. In some embodiments, each s is 4. In some embodiments, each s is 3. In some embodiments, each s is 2. In some embodiments, each s is 1. In some embodiments, each s is 0.

In some embodiments, at least one x is 2. In some embodiments, at least one x is 1. In some embodiments, at least one x is 0. In some embodiments, each x is 2. In some embodiments, each x is 1. In some embodiments, each x is 0.

In some embodiments, m is chosen from integers ranging from 2 to 128. In some embodiments, m is chosen from integers ranging from 2 to 64. In some embodiments, m is chosen from integers ranging from 2 to 32. In some embodiments, m is chosen from integers ranging from 2 to 25. In some embodiments, m is chosen from integers ranging from 2 to 16. In some embodiments, m is chosen from integers ranging from 2 to 8. In some embodiments, m is chosen from integers ranging from 2 to 4. In some embodiments, m is 4. In some embodiments, m is 3. In some embodiments, m is 2.

In some embodiments, linker groups may be chosen from groups comprising spacer groups, such spacer groups as, for example, —(CH₂)_{z}— and —O(CH₂)_{z}—, wherein z is chosen from integers ranging from 1 to 250. Other non-limiting examples of spacer groups include carbonyl groups and carbonyl-containing groups such as, for example, amide groups. A non-limiting example of a spacer group is

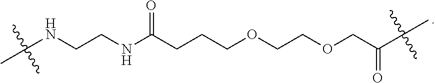 [SMILES: *NCCNC(=O)CCCOCCOCC(*)=O]

In some embodiments, L is chosen from

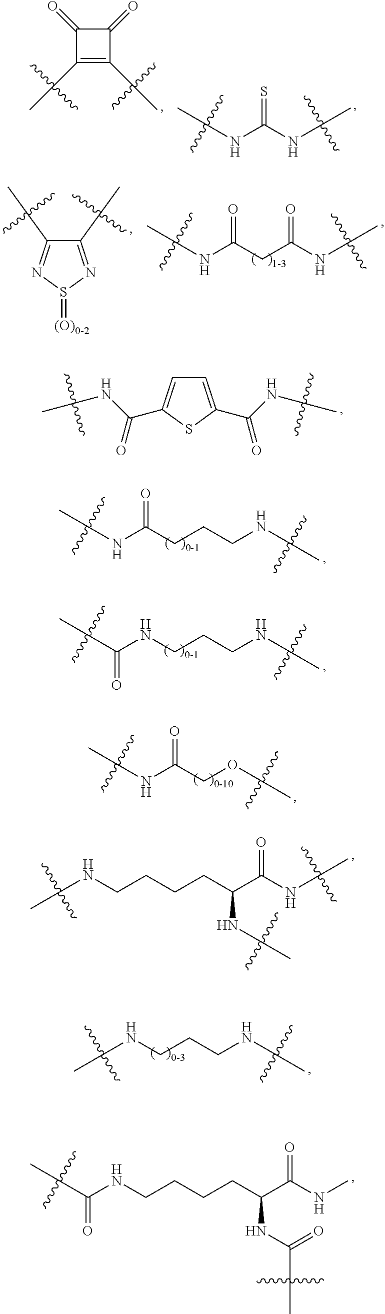

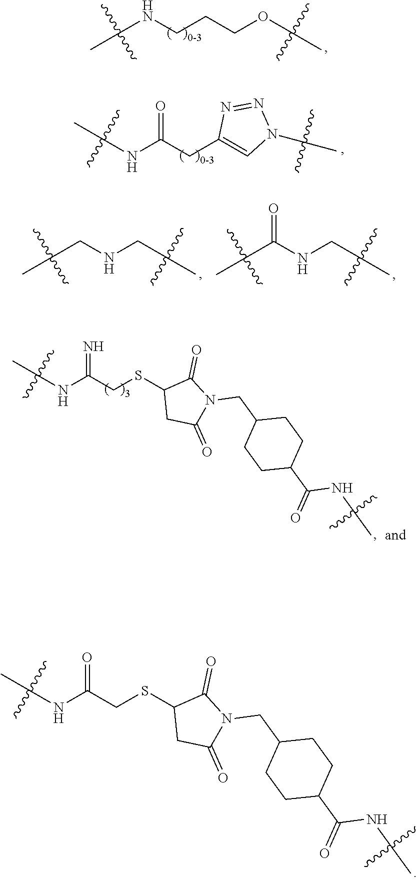

Other linker groups, such as, for example, polyethylene glycols (PEGs) and —C(═O)—NH—(CH₂)_{z}-C(═O)—NH—, wherein z is chosen from integers ranging from 1 to 250, will be familiar to those of ordinary skill in the art and/or those in possession of the present disclosure.

In some embodiments, L is

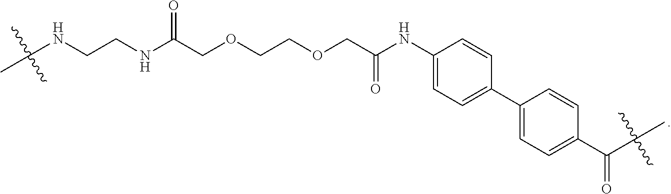 [SMILES: *NCCNC(=O)COCCOCC(=O)Nc1ccc(-c2ccc(C(*)=O)cc2)cc1]

In some embodiments, L is

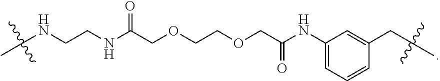 [SMILES: *Cc1cccc(NC(=O)COCCOCC(=O)NCCN*)c1]

In some embodiments, L is chosen from —C(═O)NH(CH₂)₂NH—, —CH₂NHCH₂—, and —C(═O)NHCH₂—. In some embodiments, L is —C(═O)NH(CH₂)₂NH—.

In some embodiments, L is chosen from dendrimers. In some embodiments, L is chosen from polyamidoamine (“PAMAM”) dendrimers. In some embodiments, L is chosen from PAMAM dendrimers comprising succinamic. In some embodiments, L is PAMAM GO generating a tetramer. In some embodiments, L is PAMAM G1 generating an octamer. In some embodiments, L is PAMAM G2 generating a 16-mer. In some embodiments, L is PAMAM G3 generating a 32-mer. In some embodiments, L is PAMAM G4 generating a 64-mer. In some embodiments, L is PAMAM G5 generating a 128-mer.

In some embodiments, L is chosen from

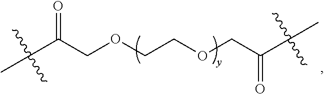

wherein y is chosen from integers ranging from 0 to 250.

In some embodiments, m is 2 and L is chosen from

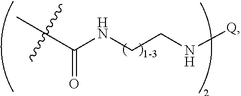

wherein Q is chosen from

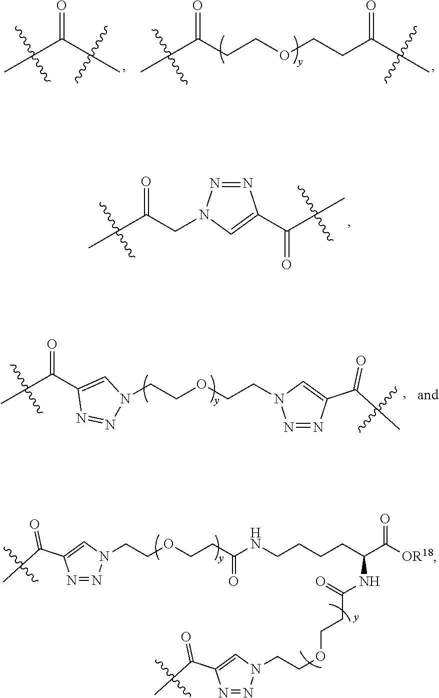

wherein R18 is chosen from H, C1-8 alkyl, C6-18 aryl, C7-19 arylalkyl, and C1-13 heteroaryl groups and each y, which may be identical or different, is independently chosen from integers ranging from 0 to 250. In some embodiments, R18 is chosen from C1-8 alkyl. In some embodiments, R18 is chosen from C7-19 arylalkyl. In some embodiments, R18 is H. In some embodiments, R18 is benzyl.

In some embodiments, L is chosen from

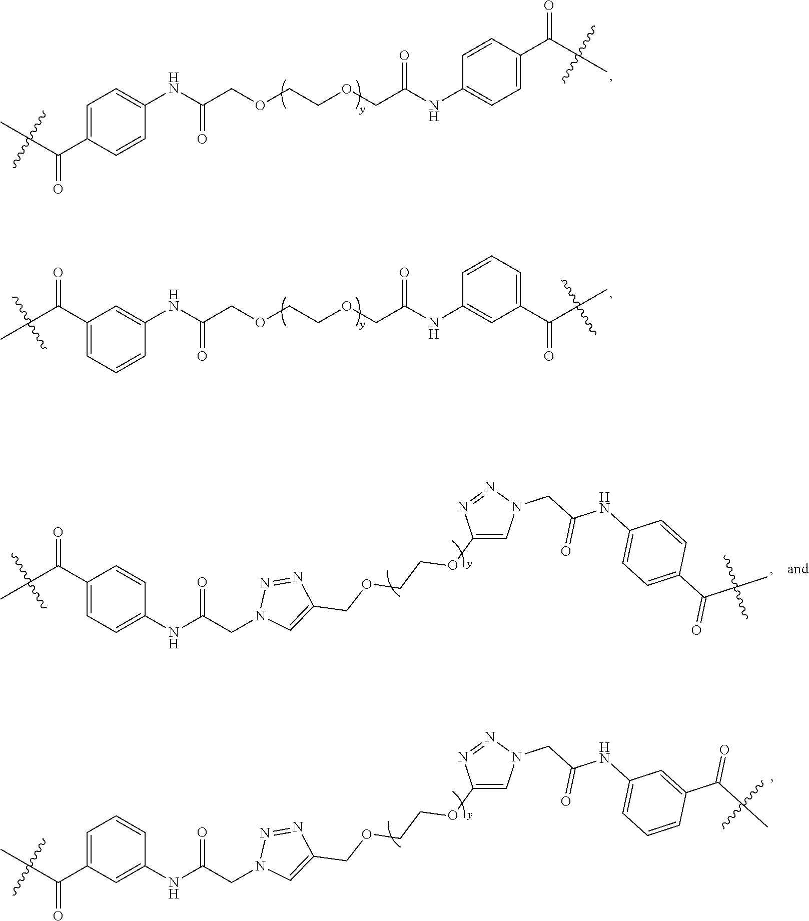

wherein y is chosen from integers ranging from 0 to 250.

In some embodiments, L is chosen from

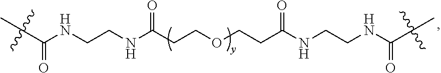

wherein y is chosen from integers ranging from 0 to 250.

In some embodiments, L is

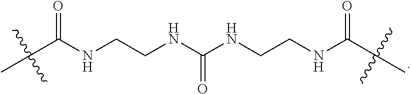 [SMILES: *C(=O)NCCNC(=O)NCCNC(*)=O]

In some embodiments, L is chosen from

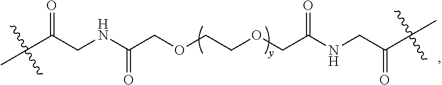

wherein y is chosen from integers ranging from 0 to 250.

In some embodiments, L is chosen from

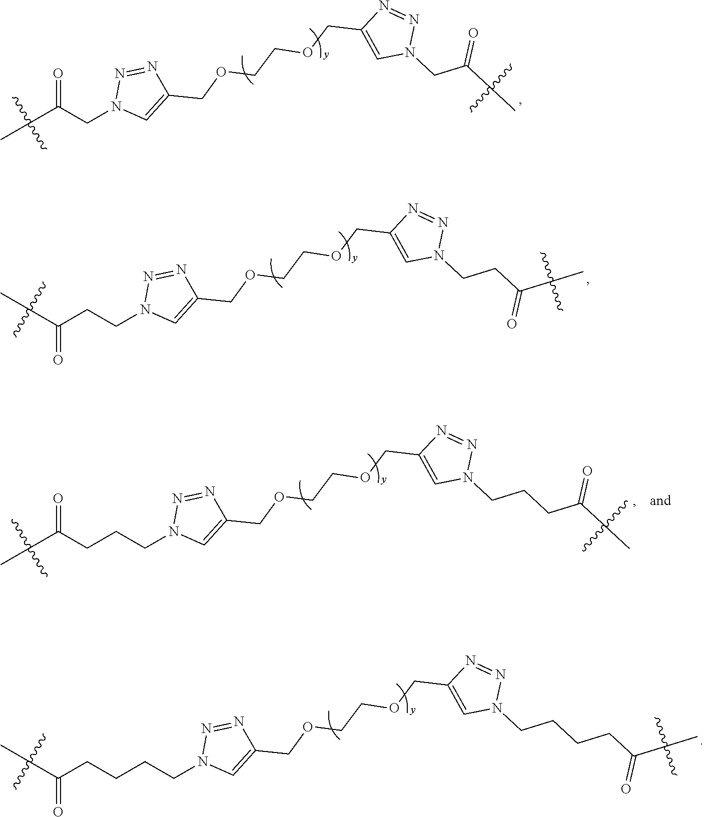

wherein y is chosen from integers ranging from 0 to 250.

In some embodiments, L is

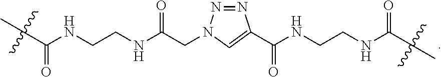 [SMILES: *C(=O)NCCNC(=O)Cn1cc(C(=O)NCCNC(*)=O)nn1]

In some embodiments, L is chosen from

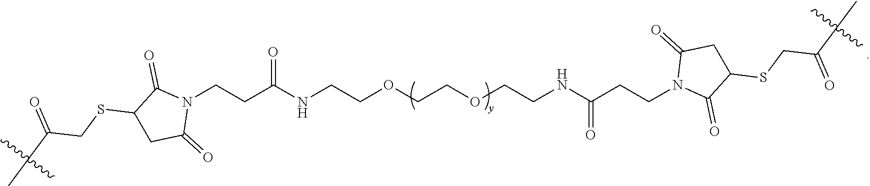

wherein y is chosen from integers ranging from 0 to 250.

In some embodiments, L is

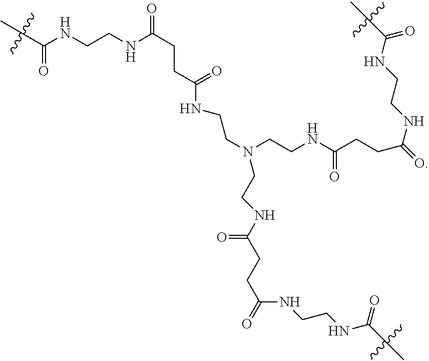 [SMILES: *C(=O)NCCNC(=O)CCC(=O)NCCN(CCNC(=O)CCC(=O)NCCNC(*)=O)CCNC(=O)CCC(=O)NCCNC(*)=O]

In some embodiments, L is

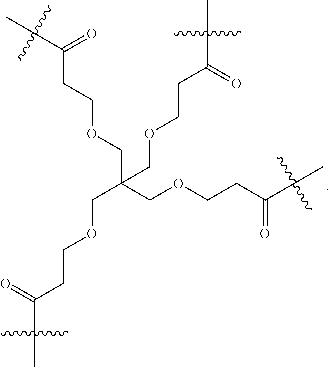 [SMILES: *C(=O)CCOCC(COCCC(*)=O)(COCCC(*)=O)COCCC(*)=O]

In some embodiments, L is

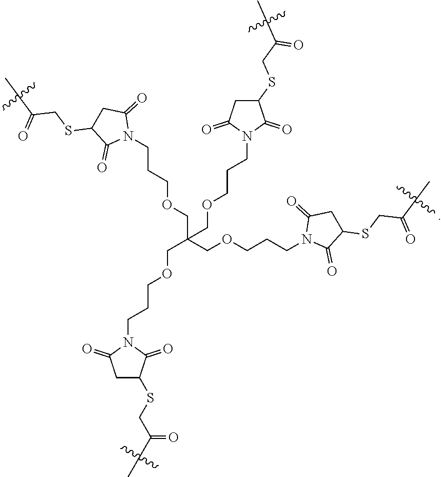 [SMILES: *C(=O)CSC1CC(=O)N(CCCOCC(COCCCN2C(=O)CC(SCC(*)=O)C2=O)(COCCCN2C(=O)CC(SCC(*)=O)C2=O)COCCCN2C(=O)CC(SCC(*)=O)C2=O)C1=O]

In some embodiments, L is chosen from

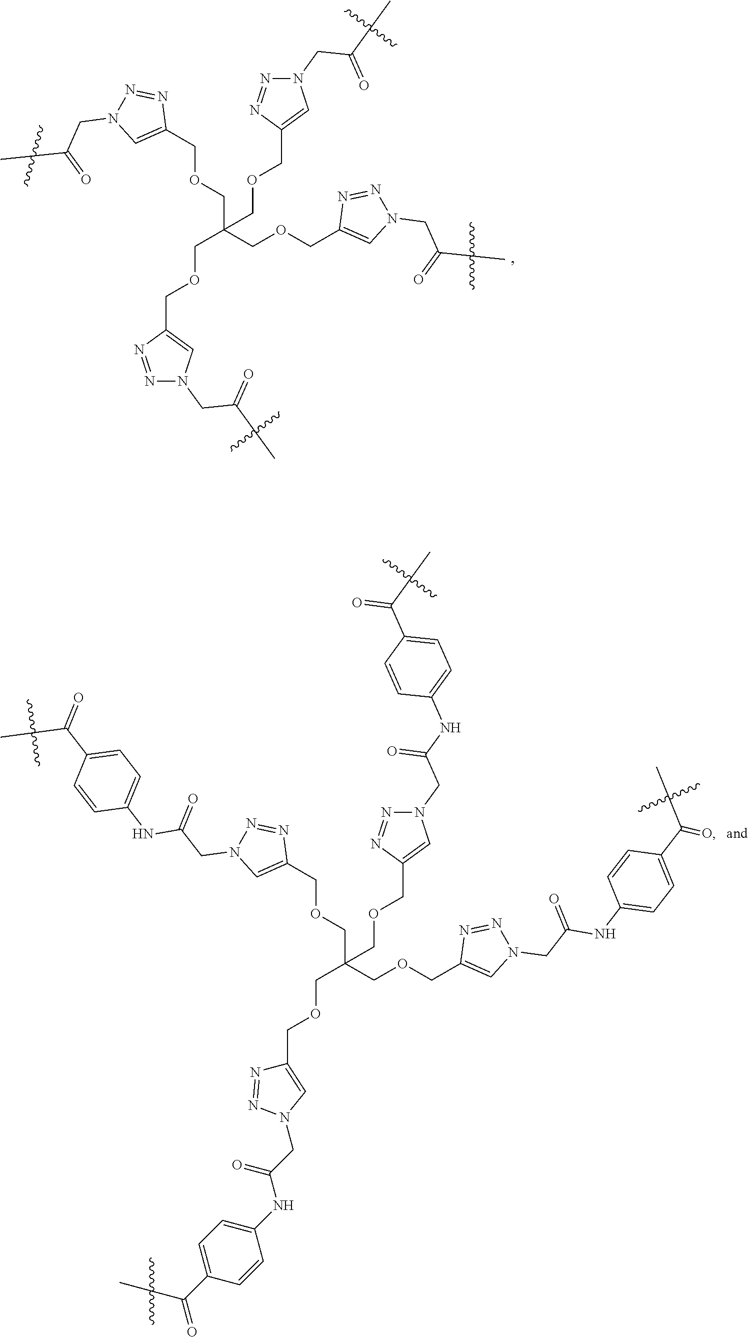 [SMILES: *C(=O)Cn1cc(COCC(COCc2cn(CC(*)=O)nn2)(COCc2cn(CC(*)=O)nn2)COCc2cn(CC(*)=O)nn2)nn1 ; *C(=O)c1ccc(NC(=O)Cn2cc(COCC(COCc3cn(CC(=O)Nc4ccc(C(*)=O)cc4)nn3)(COCc3cn(CC(=O)Nc4ccc(C(*)=O)cc4)nn3)COCc3cn(CC(=O)Nc4ccc(C(*)=O)cc4)nn3)nn2)cc1]

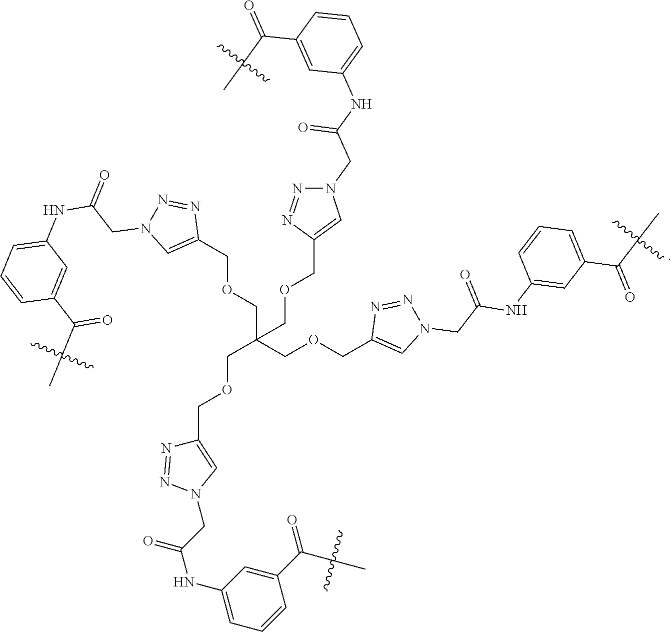 [SMILES: *C(=O)c1cccc(NC(=O)Cn2cc(COCC(COCc3cn(CC(=O)Nc4cccc(C(*)=O)c4)nn3)(COCc3cn(CC(=O)Nc4cccc(C(*)=O)c4)nn3)COCc3cn(CC(=O)Nc4cccc(C(*)=O)c4)nn3)nn2)c1]

In some embodiments, L is

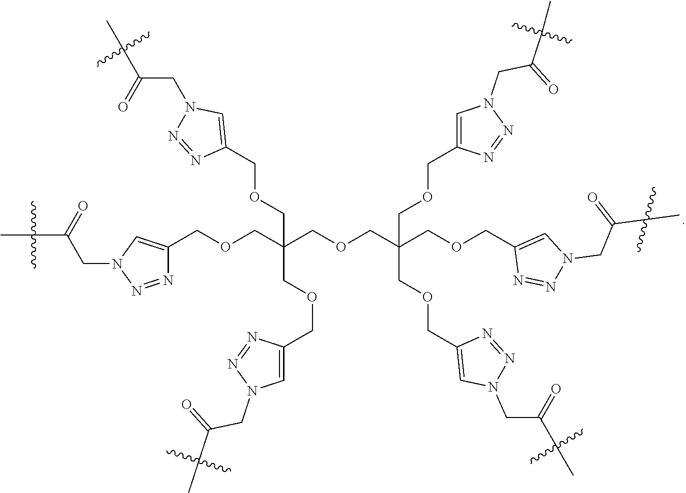 [SMILES: *C(=O)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

In some embodiments, L is chosen from

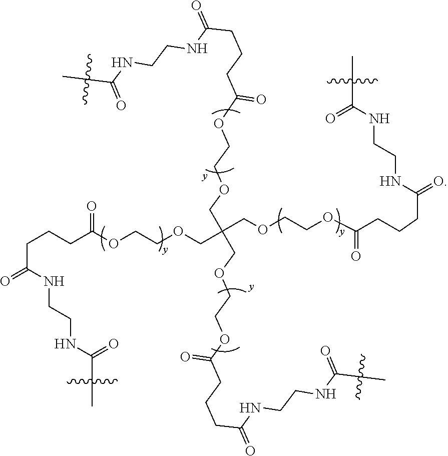

wherein y is chosen from integers ranging from 0 to 250.

In some embodiments, L is chosen from

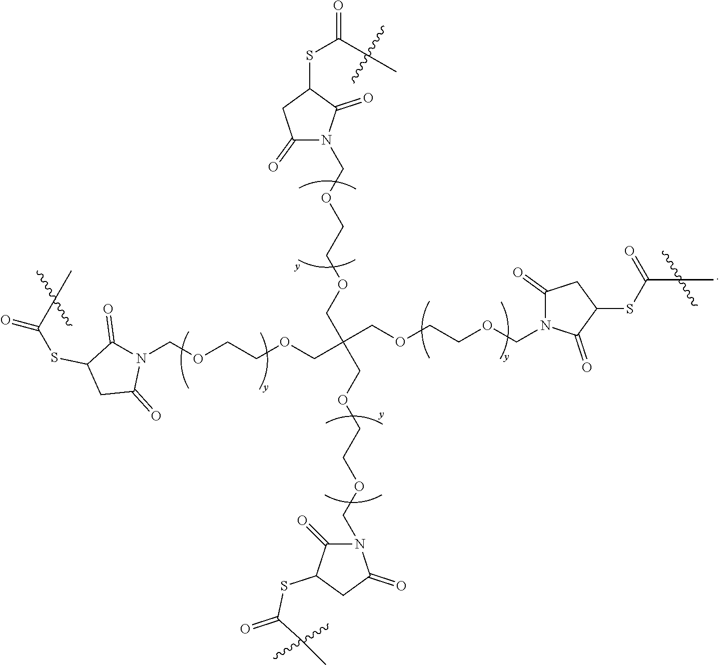

wherein y is chosen from integers ranging from 0 to 250.

In some embodiments, L is

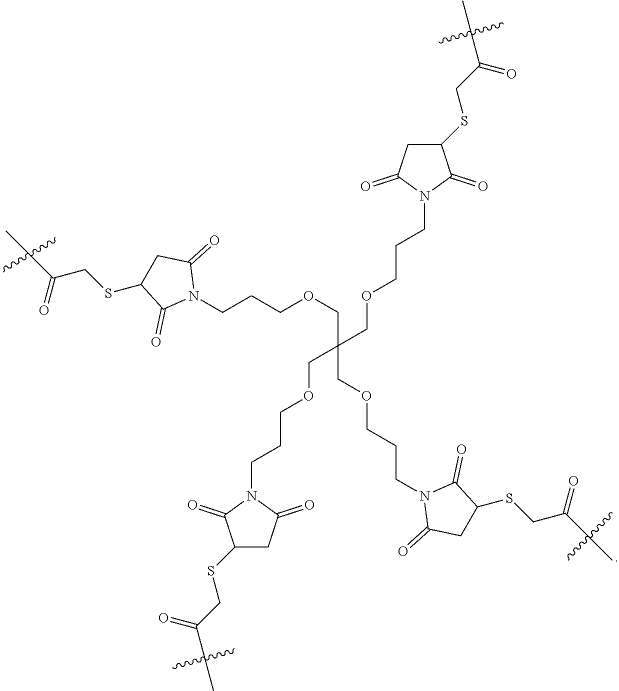 [SMILES: *C(=O)CSC1CC(=O)N(CCCOCC(COCCCN2C(=O)CC(SCC(*)=O)C2=O)(COCCCN2C(=O)CC(SCC(*)=O)C2=O)COCCCN2C(=O)CC(SCC(*)=O)C2=O)C1=O]

In some embodiments, L is

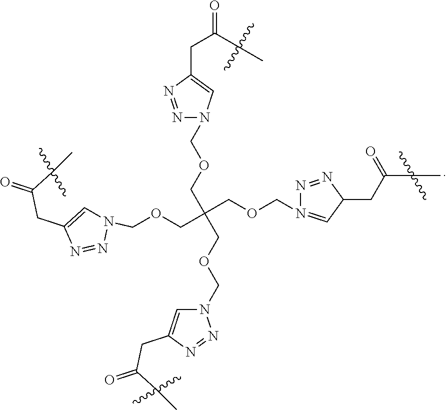

In some embodiments y is chosen from integers ranging from 0 to 200. In some embodiments, y is chosen from integers ranging from 0 to 150. In some embodiments, y is chosen from integers ranging from 0 to 100. In some embodiments, y is chosen from integers ranging from 0 to 50. In some embodiments, y is chosen from integers ranging from 0 to 30. In some embodiments, y is chosen from integers ranging from 0 to 15. In some embodiments, y is chosen from integers ranging from 0 to 10. In some embodiments, y is chosen from integers ranging from 0 to 5. In some embodiments, y is 117. In some embodiments, y is 25. In some embodiments, y is 21. In some embodiments, y is 17. In some embodiments y is 13. In some embodiments, y is 10. In some embodiments, y is 8. In some embodiments, y is 6. In some embodiments, y is 5. In some embodiments, y is 4. In some embodiments, y is 3. In some embodiments, y is 2. In some embodiments, y is 1. In some embodiments, y is 0.

In some embodiments, at least one compound is chosen from compounds of Formula (I), wherein each R¹ is identical, each R² is identical, each R³ is identical, each R⁴ is identical, each R⁵ is identical, each X¹ is identical, each j is identical, each k is identical, each n is identical, and each s is identical. In some embodiments, at least one compound is chosen from compounds of Formula (I), wherein said compound is symmetrical.

In some embodiments, at least one compound is chosen from compounds having the following Formula:

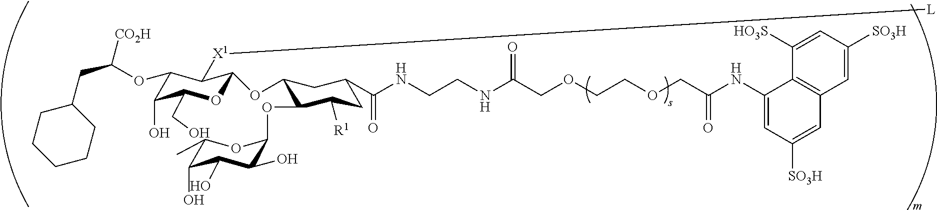

and pharmaceutically acceptable salts thereof.

In some embodiments, at least one compound is chosen from compounds having the following Formulae:

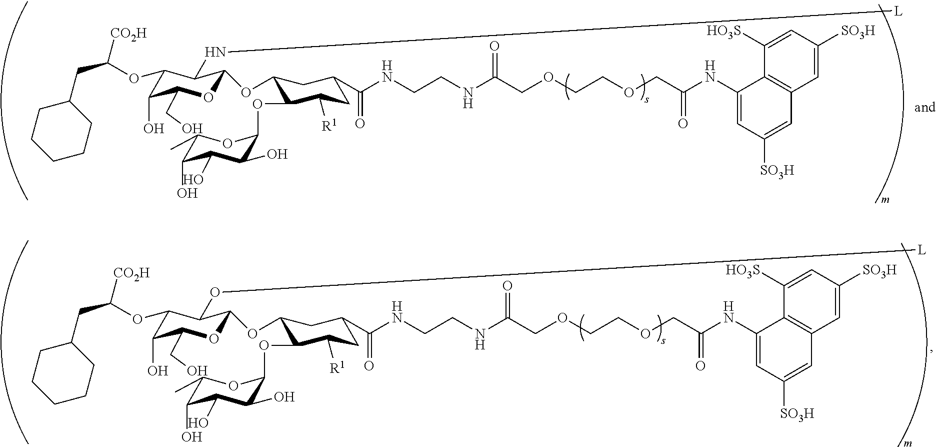

and pharmaceutically acceptable salts of any of the foregoing.

In some embodiments, at least one compound is chosen from compounds having the following Formulae:

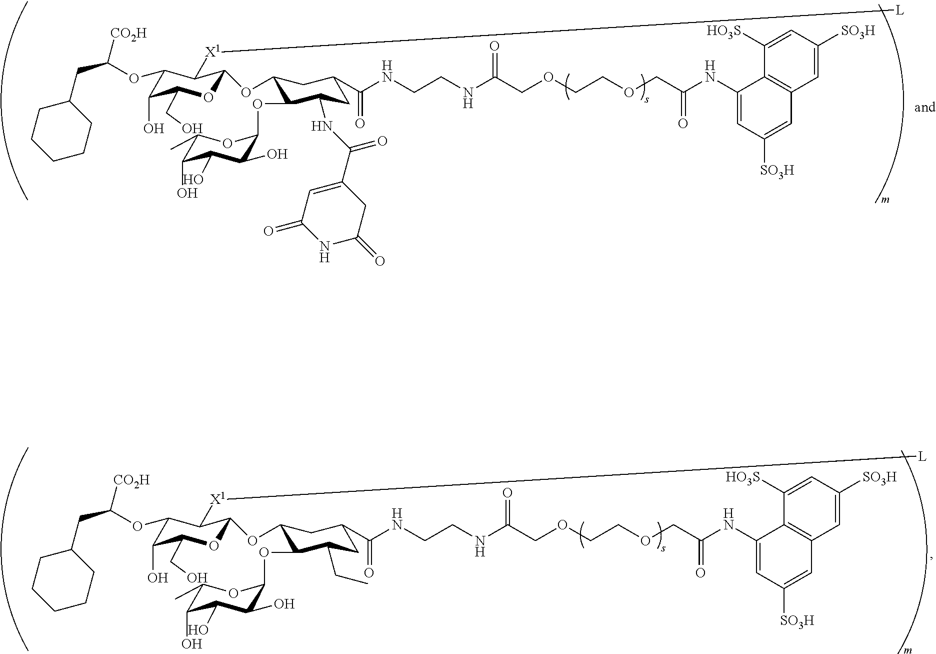

and pharmaceutically acceptable salts of any of the foregoing.

In some embodiments, at least one compound is chosen from compounds having the following Formulae:

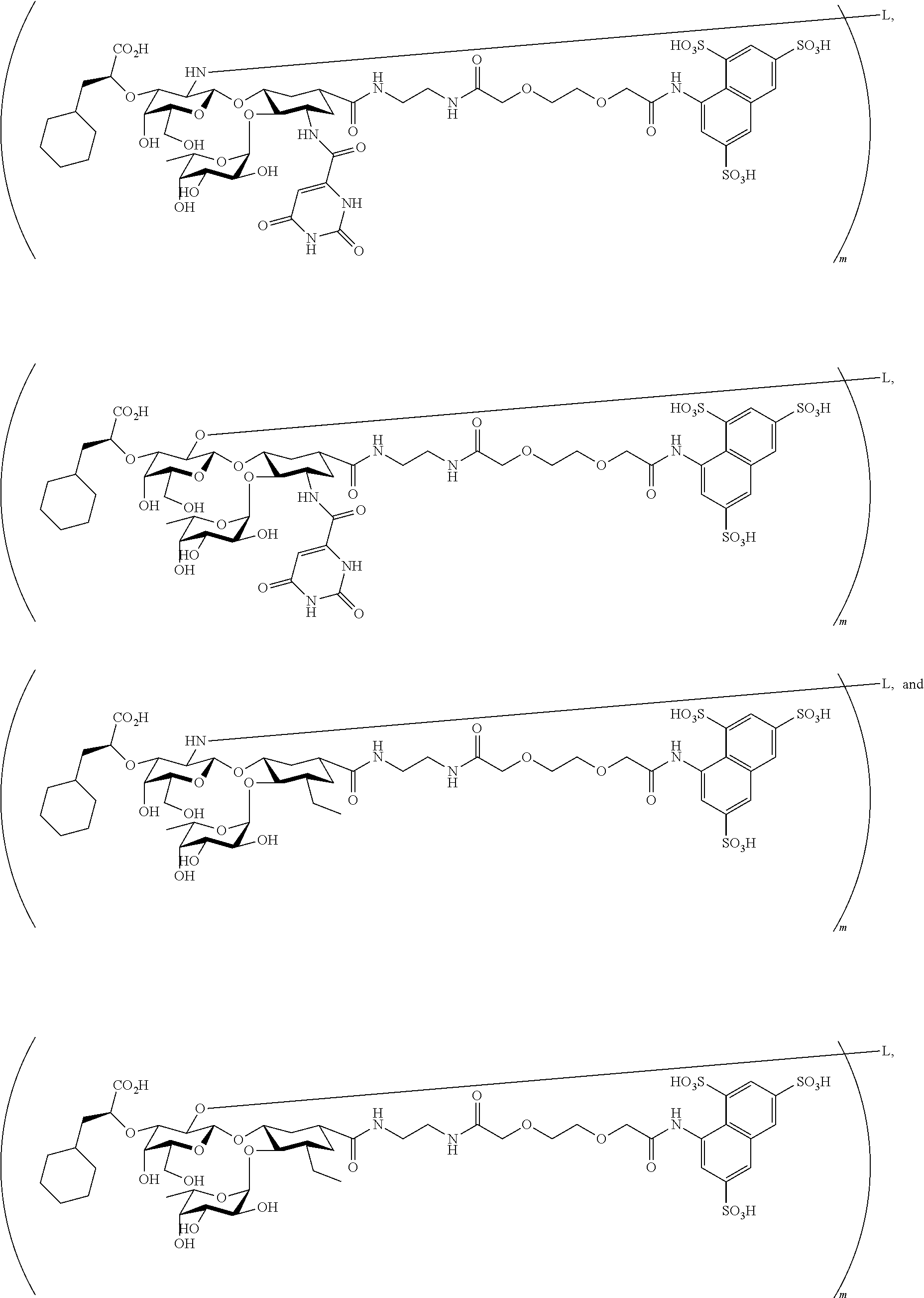

and pharmaceutically acceptable salts of any of the foregoing.

In some embodiments, at least one compound is chosen from compounds having the Formula:

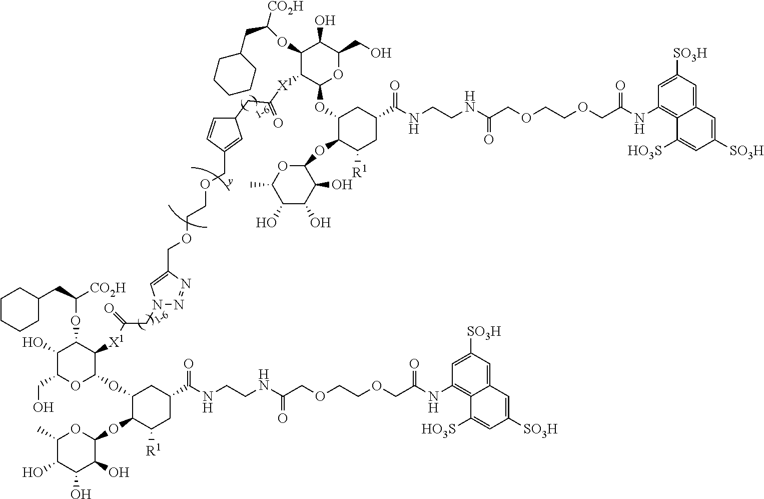

and pharmaceutically acceptable salts thereof.

In some embodiments, at least one compound is chosen from compounds having the Formulae:

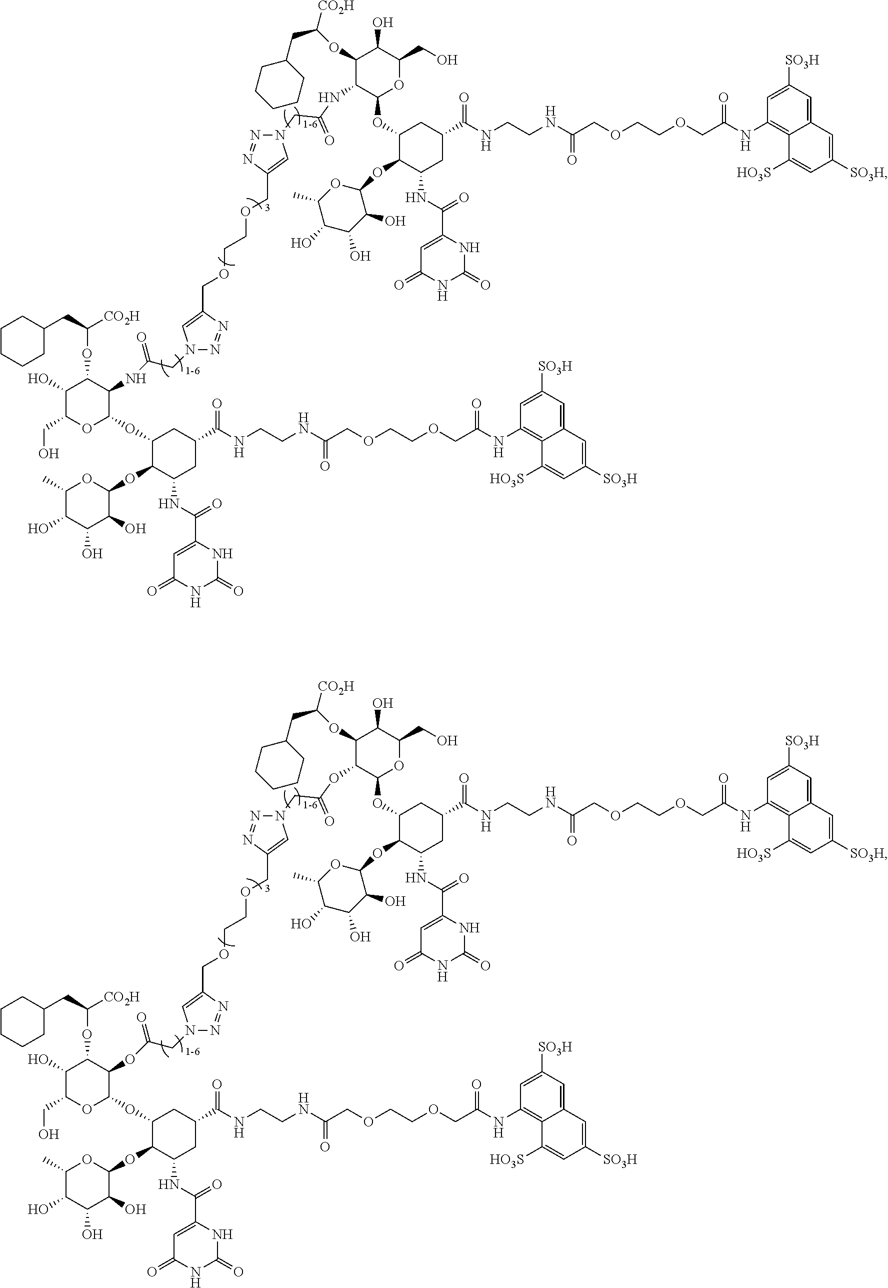

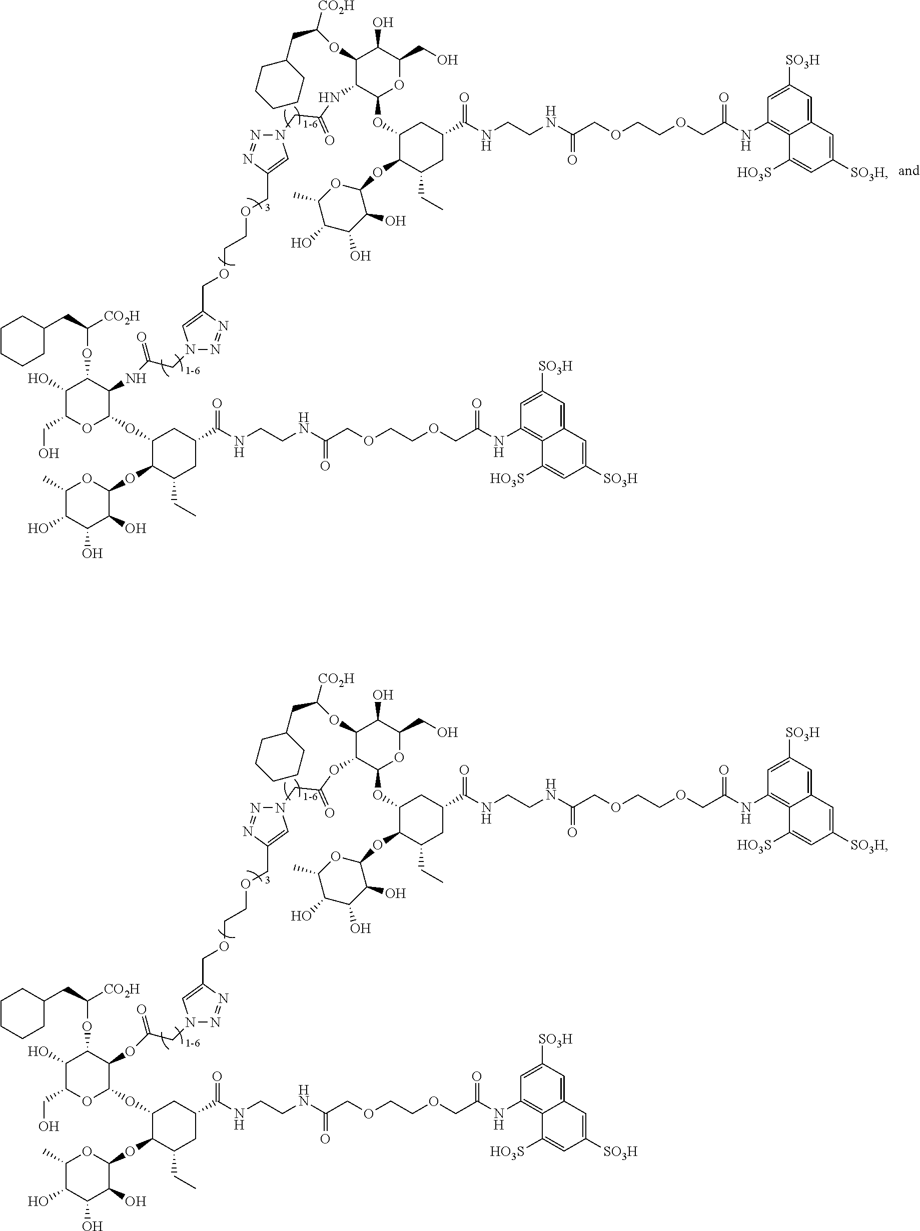

and pharmaceutically acceptable salts of any of the foregoing.

In some embodiments, at least one compound is chosen from compounds having the following Formulae:

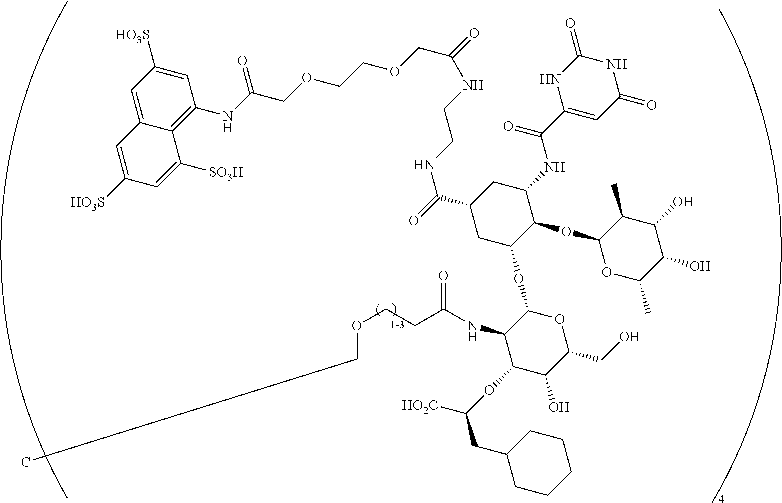

and pharmaceutically acceptable salts thereof.

In some embodiments, at least one compound is chosen from compounds having the following Formulae:

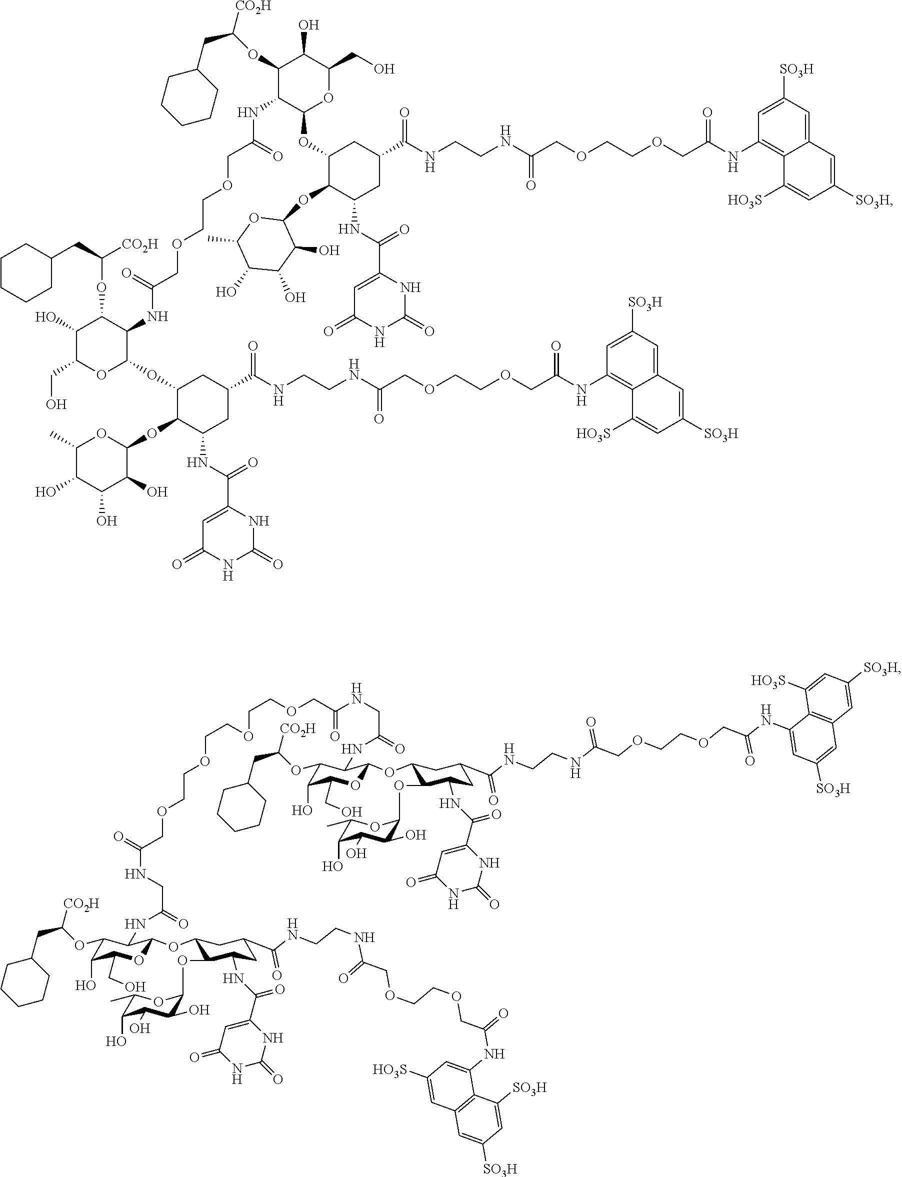

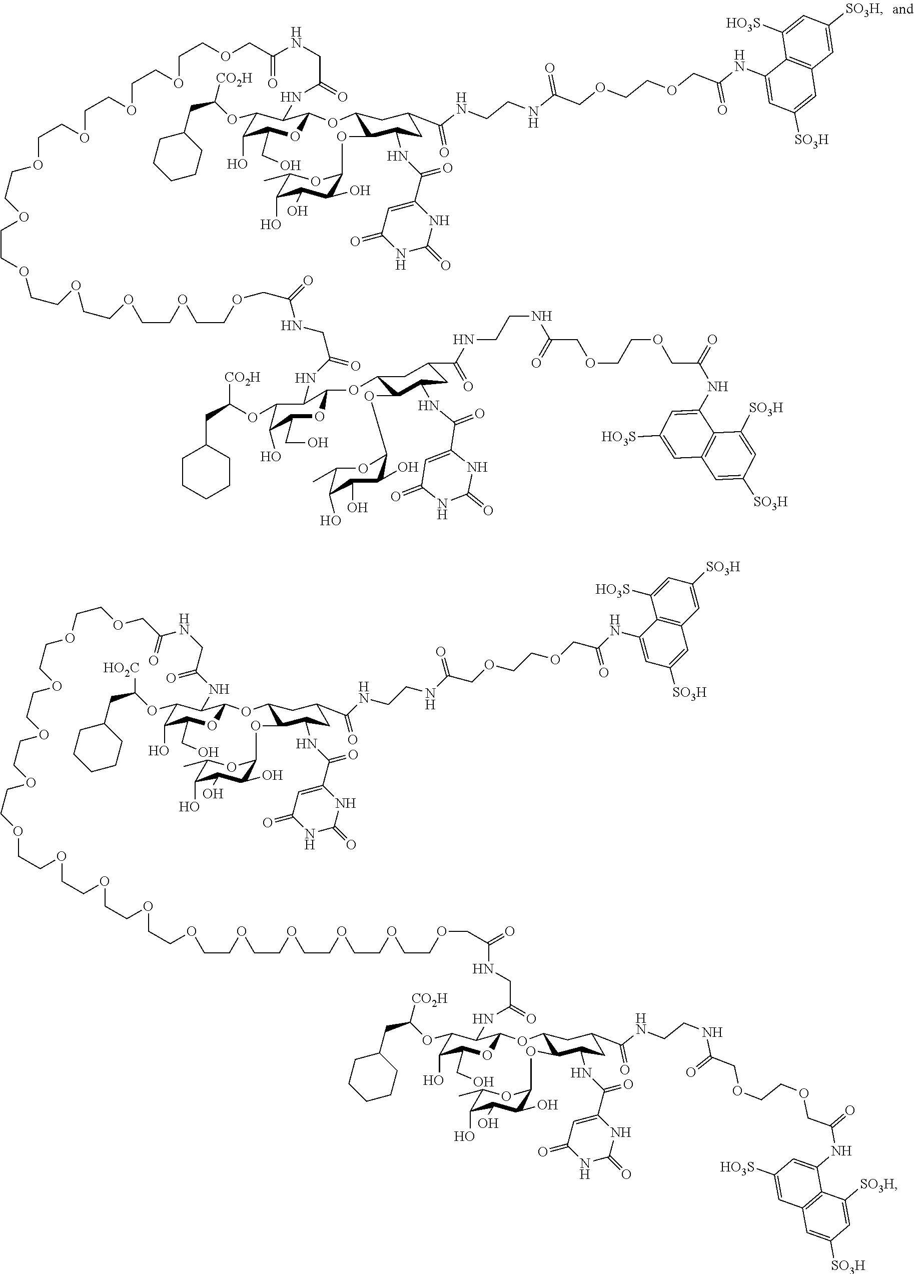

and pharmaceutically acceptable salts of any of the foregoing.

In some embodiments, at least one compound is chosen from compounds having the following Formulae:

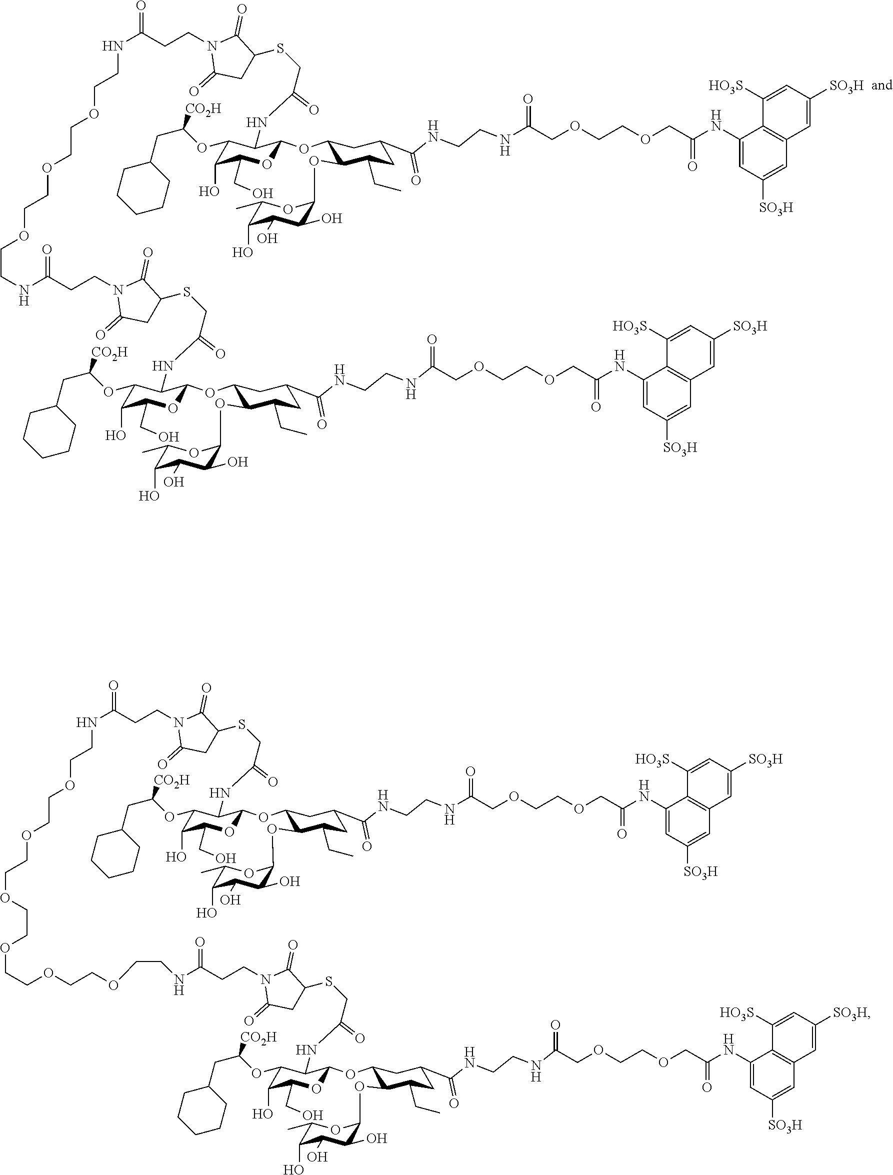

and pharmaceutically acceptable salts of any of the foregoing.

In some embodiments, at least one compound is chosen from compounds having the Formulae:

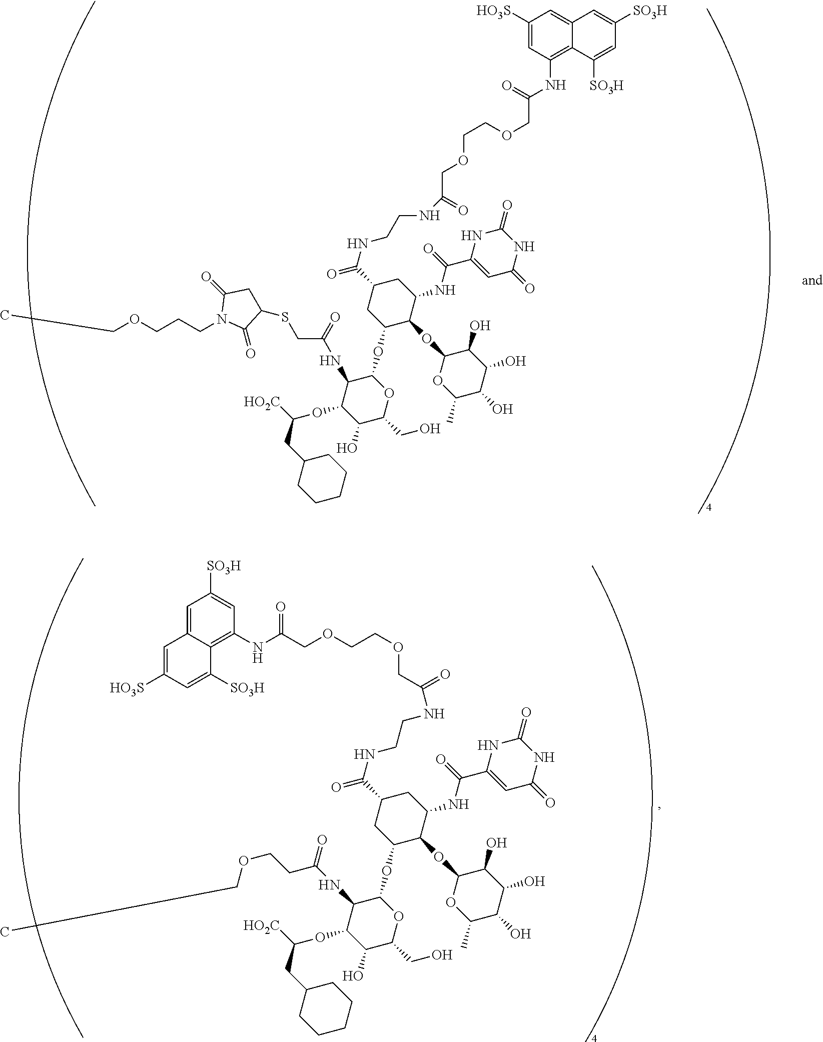

and pharmaceutically acceptable salts of any of the foregoing.

In some embodiments, at least one compound is chosen from compounds having the Formulae:

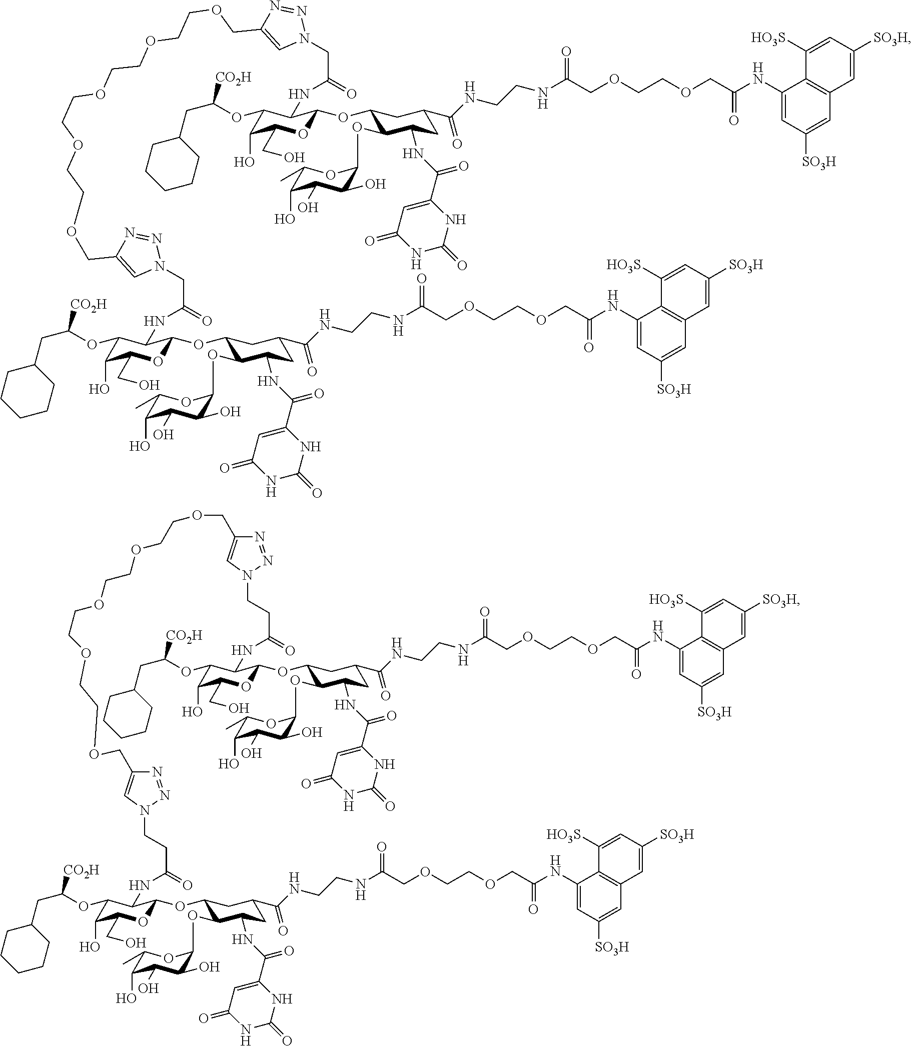

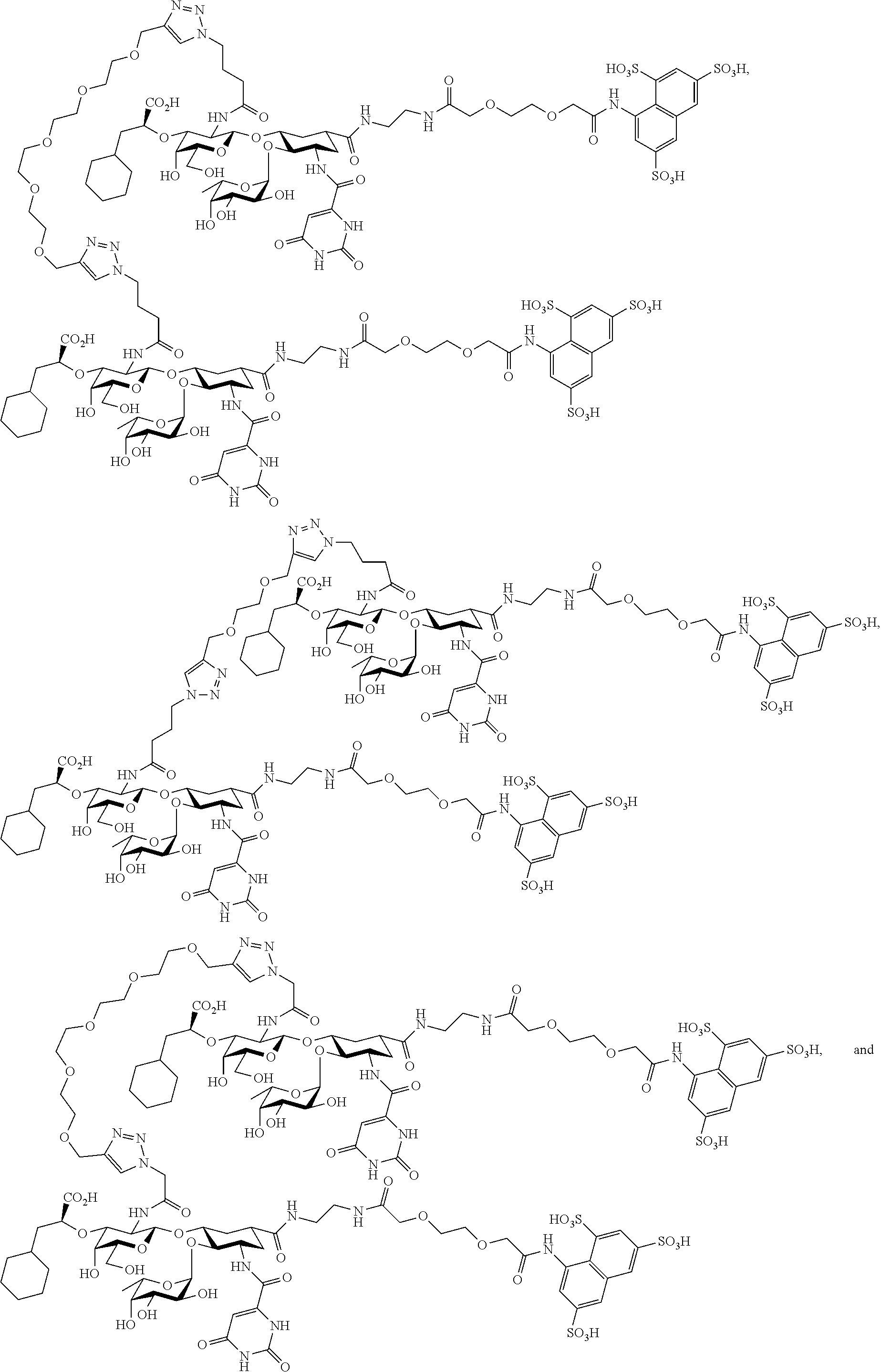

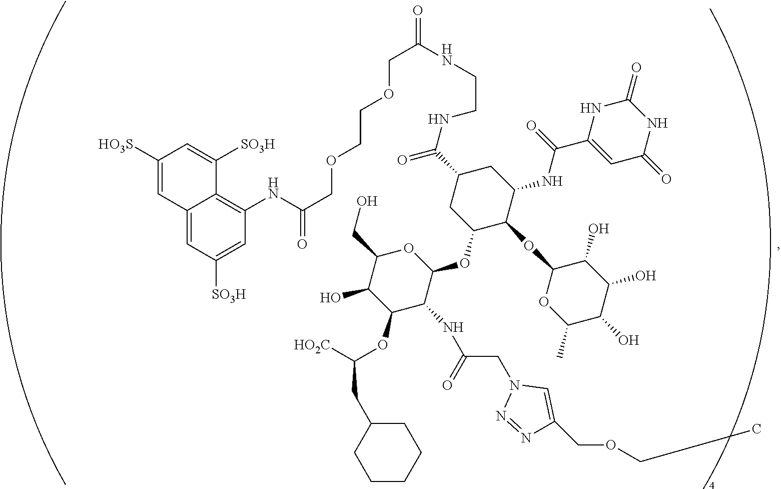

and pharmaceutically acceptable salts of any of the foregoing.

In some embodiments, at least one compound is chosen from compounds having the Formulae:

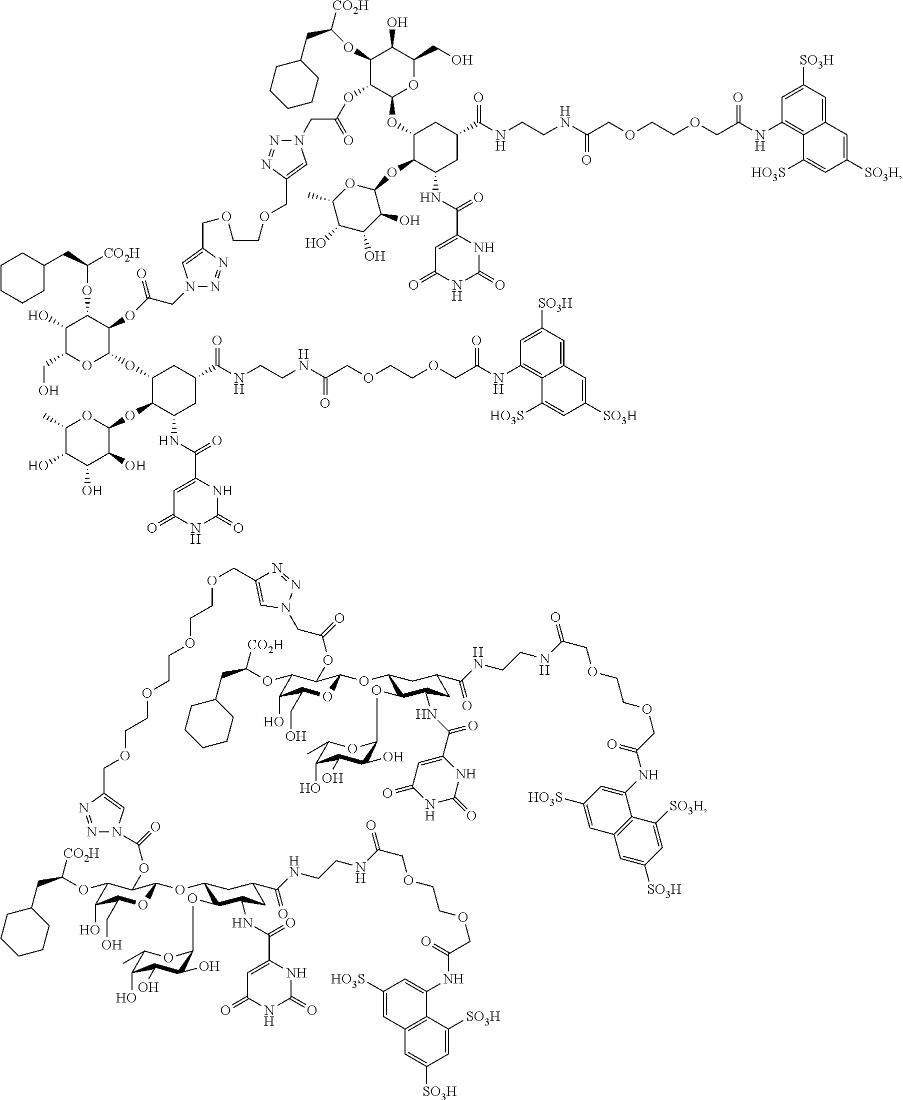

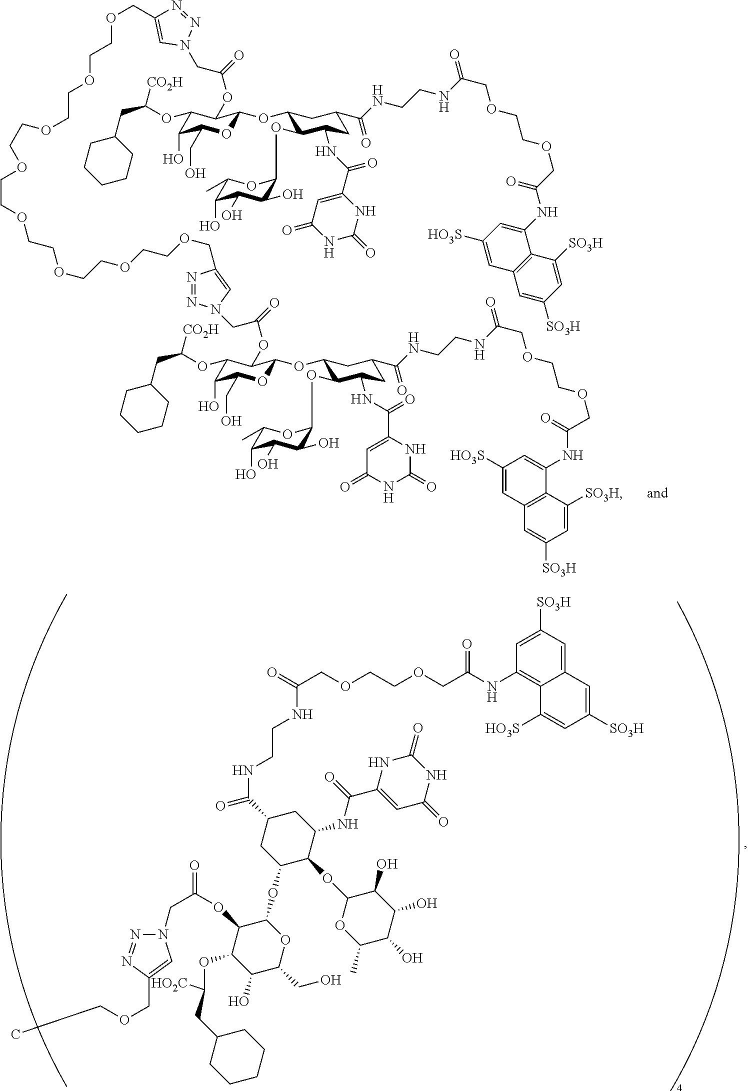

and pharmaceutically acceptable salts of any of the foregoing.

In some embodiments, at least one compound is chosen from compounds having the following Formulae:

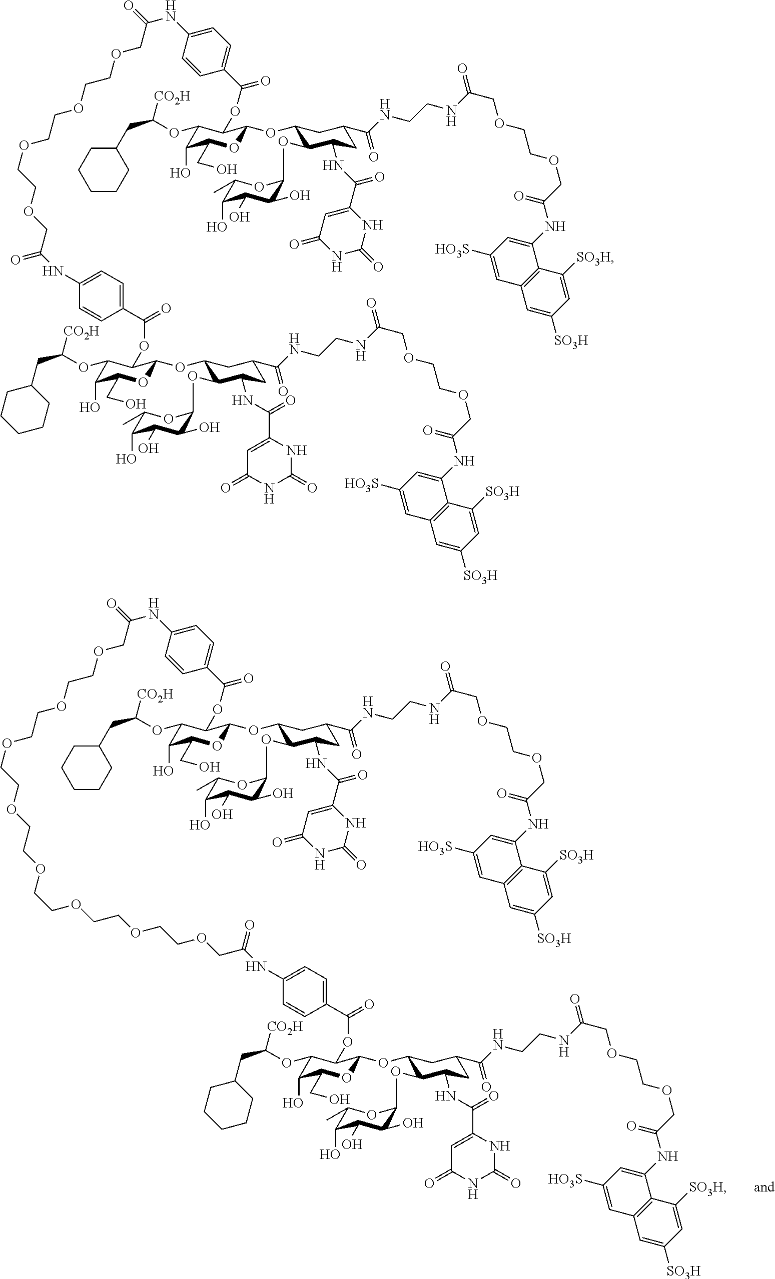

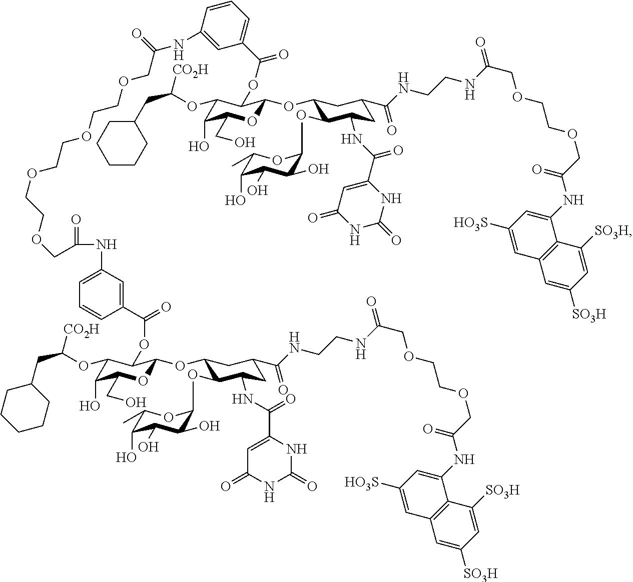

and pharmaceutically acceptable salts of any of the foregoing.

In some embodiments, at least one compound is chosen from compounds having the following Formulae:

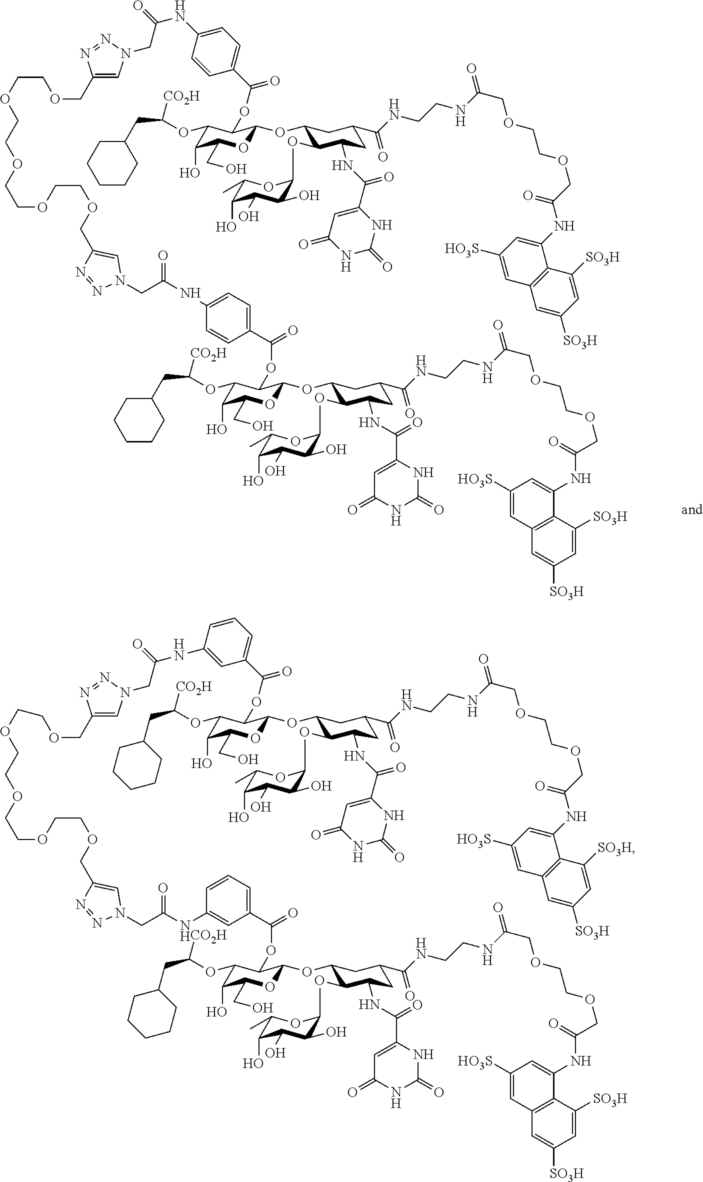

and pharmaceutically acceptable salts of any of the foregoing.

In some embodiments, at least one compound is chosen from compounds having the Formulae:

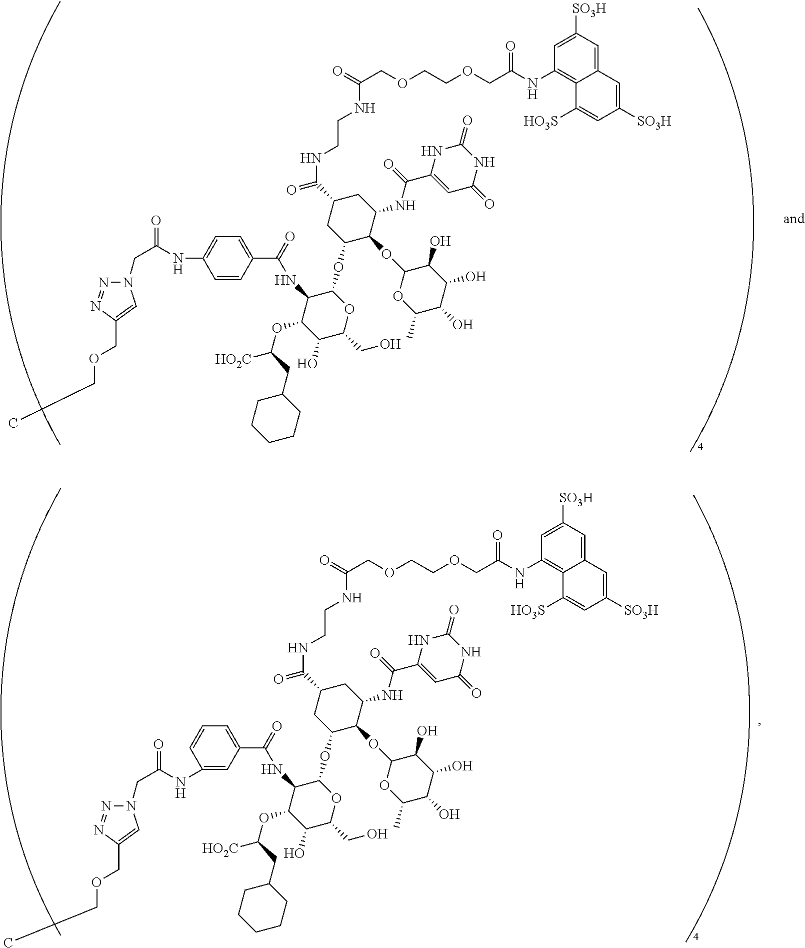

and pharmaceutically acceptable salts of any of the foregoing.

In some embodiments, at least one compound is chosen from compounds having the following Formula:

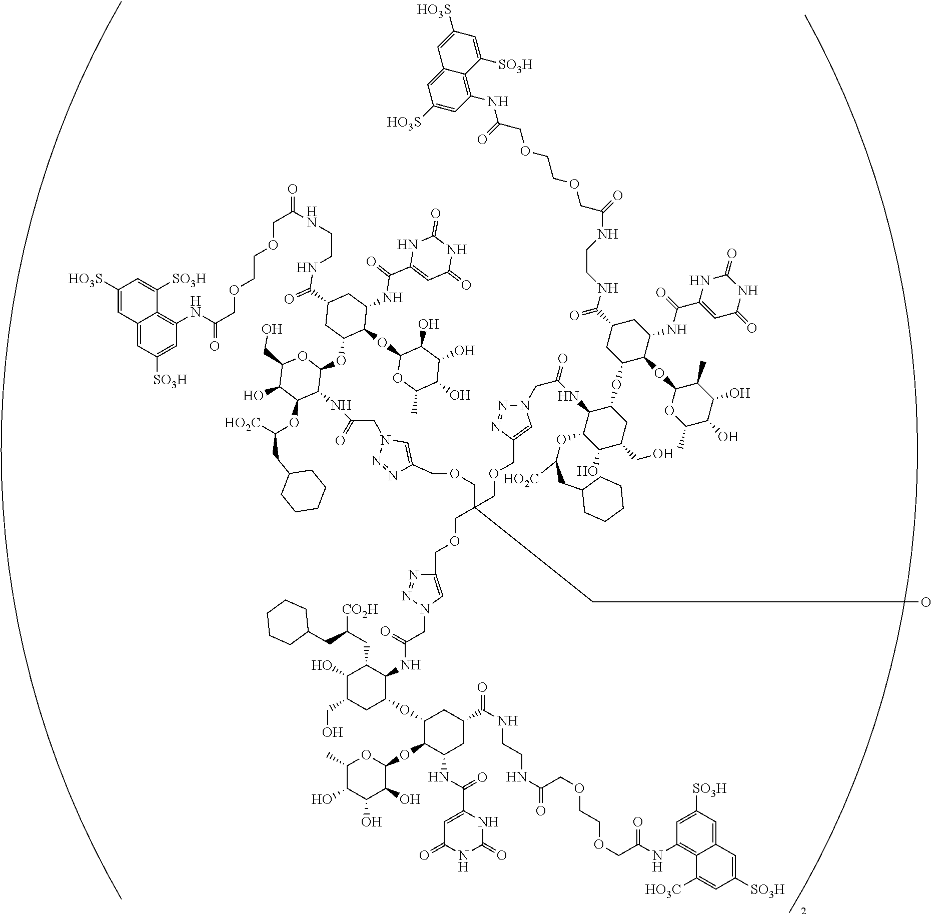

and pharmaceutically acceptable salts thereof.

Also provided are pharmaceutical compositions comprising at least one compound of Formula (I) as described herein. Such pharmaceutical compositions are described in greater detail herein. These compounds and compositions may be used in the methods described herein.

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present invention are generally performed, unless otherwise indicated, according to conventional methods well known in the art and as described in various general and more specific references that are cited and discussed throughout this specification.

Chemistry terms used herein are used according to conventional usage in the art, as exemplified by “The McGraw-Hill Dictionary of Chemical Terms,” Parker S., Ed., McGraw-Hill, San Francisco, Calif. (1985).

All of the publications, patents and published patent applications referred to in this application are specifically incorporated by reference herein.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to”. “Including” and “including but not limited to” are used interchangeably.

Whenever a term in the specification is identified as a range (e.g., C₁₋₄ alkyl), the range independently discloses and includes each element of the range. As a non-limiting example, C₁₋₄ alkyl includes, independently, C₁ alkyls, C₂ alkyls, C₃ alkyls, and C₄ alkyls.

The term “at least one” refers to one or more, such as one, two, etc. For example, the term “at least one C₁₋₄ alkyl” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “cycloalkyl” includes saturated monocyclic or polycyclic hydrocarbon group, which may include fused or bridged ring systems. Non-limiting examples of cycloalkyl groups include cyclopropyl, cyclobutyl, cyclopentyl, cyclohexyl, cycloheptyl, cyclooctyl, adamantyl, and norbornyl. Unless otherwise stated specifically in the specification, a cycloalkyl group may be optionally substituted.

The term “cycloalkylalkyl” includes cycloalkyl groups, as described herein, appended to the parent molecular moiety through an alkyl group, as defined herein. Non-limiting examples of cycloalkylalkyl groups include cyclopropylmethyl and cyclohexylmethyl. Unless stated otherwise specifically in the specification, a cycloalkylalkyl group may be optionally substituted.

The term “aryl” includes hydrocarbon ring system group comprising 6 to 18 carbon ring atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “fused” includes any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

The term “glycomimetic” includes any naturally occurring or non-naturally occurring carbohydrate compound in which at least one substituent has been replaced, or at least one ring has been modified (e.g., substitution of carbon for a ring oxygen), to yield a compound that is not fully carbohydrate.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2 trifluoroethyl, 1,2 difluoroethyl, 3 bromo 2 fluoropropyl, and 1,2 dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2 difluoroethenyl, 3 bromo 2 fluoropropenyl, and 1,2 dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2 difluoroethynyl, 3 bromo 2 fluoropropynyl, and 1,2 dibromoethynyl. A “fluoroalkynyl” is a haloalkynyl wherein at least one halogen is fluoro. Unless stated otherwise specifically in the specification, a haloalkynyl group may be optionally substituted.

The term “heterocyclyl” or “heterocyclic ring” includes 3- to 18-membered saturated or partially unsaturated non-aromatic ring groups comprising 2 to 12 ring carbon atoms and 1 to 6 ring heteroatom(s) each independently chosen from N, O, and S. Unless stated otherwise specifically in the specification, the heterocyclyl groups may be a monocyclic, bicyclic, tricyclic or tetracyclic ring system, which may include fused or bridged ring systems; and the nitrogen, carbon or sulfur atoms in the heterocyclyl group may be optionally oxidized; the nitrogen atom may be optionally quaternized; and the heterocyclyl group may be partially or fully saturated. Non-limiting examples include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

The term “pharmaceutically acceptable salts” includes both acid and base addition salts. The acid addition salt form of a compound that occurs in its free form as a base can be obtained by treating said free base form with an appropriate acid such as an inorganic acid or an organic acid. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Compounds containing acidic protons may be converted into their base addition salt form by treatment with appropriate organic and inorganic bases. Non-limiting examples of pharmaceutically acceptable base addition salts include sodium, potassium, lithium, ammonium (substituted and unsubstituted), calcium, magnesium, iron, zinc, copper, manganese, aluminum salts, N-methyl-D-glucamine salts, hydrabamine salts, and salts with amino acids such as, arginine, lysine and the like. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

This application contemplates all the isomers of the compounds disclosed herein. “Isomer” as used herein includes optical isomers (such as stereoisomers, e.g., enantiomers and diastereoisomers), geometric isomers (such as Z (zusammen) or E (entgegen) isomers), and tautomers. The present disclosure includes within its scope all the possible geometric isomers, e.g., Z and E isomers (cis and trans isomers), of the compounds as well as all the possible optical isomers, e.g. diastereomers and enantiomers, of the compounds. Furthermore, the present disclosure includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g. fractional crystallization, may be used.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms.

Biological activity of the selectin modulators described herein may be determined, for example, by performing at least one in vitro and/or in vivo study routinely practiced in the art and described herein or in the art.

In vitro assays include without limitation binding assays, immunoassays, competitive binding assays, and cell based activity assays. Selectin modulators as described above are capable, for example, of inhibiting selectin-mediated cell adhesion. This ability may generally be evaluated using any of a variety of in vitro assays designed to measure the effect on adhesion between selectin-expressing cells (e.g., adhesion between leukocytes or tumor cells and platelets or endothelial cells). For example, such cells may be plated under standard conditions that, in the absence of modulator, permit cell adhesion. In general, a selectin modulator is an inhibitor of selectin-mediated cell adhesion if contact of the test cells with the selectin modulator results in a discernible inhibition of cell adhesion. For example, in the presence of selectin modulators (e.g., micromolar levels), disruption of adhesion between leukocytes or tumor cells and platelets or endothelial cells may be determined visually within approximately several minutes, by observing the reduction of cells interacting with one another.

Selectin modulators may also be used in vitro, e.g., within a variety of well-known cell culture and cell separation methods. For example, modulators may be linked to the interior surface of a tissue culture plate or other cell culture support, for use in immobilizing selectin-expressing cells for screens, assays and growth in culture. Such linkage may be performed by any suitable technique. Selectin modulators may also be used, for example, to facilitate cell identification and sorting in vitro, permitting the selection of cells expressing a selectin (or different selectin levels). In some embodiments, the modulator(s) for use in such methods are linked to a detectable marker. Suitable markers are well known in the art and include radionuclides, luminescent groups, fluorescent groups, enzymes, dyes, constant immunoglobulin domains and biotin. In some embodiments, a selectin modulator linked to a fluorescent marker, such as fluorescein, is contacted with the cells, which are then analyzed by fluorescence activated cell sorting (FACS).

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein, methods for characterizing selectin modulators include animal model studies. Non-limiting examples of animal models for liquid cancers used in the art include multiple myeloma (see. e.g., DeWeerdt, Nature 480:S38-S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 Apr. 1; 23(7): 877-889). Animal models for acute lymphoblastic leukemia (ALL) have been used by persons of ordinary skill in the art for more than two decades. Numerous exemplary animal models for solid tumor cancers are routinely used and are well known to persons of ordinary skill in the art.

In certain aspects, the compounds and compositions as described herein can be used to treat patients suffering from a condition associated with a selectin-mediated function. A variety of conditions are associated with a selectin-mediated function. Such conditions include, for example, tissue transplant rejection including graft versus host disease (GVHD), platelet-mediated diseases (e.g., atherosclerosis and clotting), sickle cell disease, vaso-occlusive disorders, sinusoidal obstruction syndrome, epilepsy, hyperactive coronary circulation, acute leukocyte-mediated lung injury (e.g., adult respiratory distress syndrome (ARDS)), Crohn's disease, inflammatory diseases (e.g., inflammatory bowel disease), autoimmune diseases (MS, myasthenia gravis), infection, cancer including blood cancers such as AML, ALL, CML, and MM (and metastasis), thrombosis, wounds (and wound-associated sepsis), burns, spinal cord damage, digestive tract mucous membrane disorders (gastritis, ulcers), osteoporosis, rheumatoid arthritis, osteoarthritis, asthma, allergy, psoriasis, septic shock, traumatic shock, stroke, nephritis, atopic dermatitis, frostbite injury, adult dyspnoea syndrome, ulcerative colitis, systemic lupus erythematosus, diabetes and reperfusion injury following ischaemic episodes. Selectin modulators may also be administered to a patient prior to heart surgery to enhance recovery. Other uses include pain management, prevention of restinosis associated with vascular stenting, and for undesirable angiogenesis, e.g., associated with cancer.

In certain aspects, the compounds and compositions as described herein can be used to treat patients suffering from sickle cell disease (SCD) or complications associated therewith. Sickle cell disease is an inheritable hematological disorder based on a mutation in the β-globin gene of hemoglobin. Upon deoxygenation, this mutated hemoglobin polymerizes and causes a shape change (sickling) of the red blood cell. This change in red blood cells leads to obstruction of blood vessels (vaso-occlusion) causing a wide variety of complications such as stroke, pulmonary hypertension, end-organ disease and death. Vaso-occlusive phenomena and hemolysis are clinical hallmarks of SCD. Vaso-occlusion results in recurrent painful episodes (sometimes called sickle cell crisis or vaso-occlusive crisis) and a variety of serious organ system complications among which, infection, acute chest syndrome, stroke, splenic sequestration are among the most debilitating. Vaso-occlusion accounts for 90% of hospitalizations in children with SCD, and it can ultimately lead to life-long disabilities and/or early death.

In addition to the fatal or potentially fatal complications, there are serious non-fatal complications of sickle cell disease such as pain. The severity of the pain may vary, but normally requires some form of medical attention. Hospitalization may be necessary.

In the U.S. alone, approximately 70,000-80,000 people suffer from sickle cell disease. Sickle cell disease is estimated to affect one of every 1,300 infants in the general population, and one of every 400 of African descent. Currently, there is no cure for sickle cell disease. The disease is chronic and lifelong. Life expectancy is typically shortened.

In certain aspects, the compounds and compositions as described herein can be used to treat patients suffering from graft versus host disease or complications associated therewith Graft-versus-host disease, GVHD, is an immunological disorder that is the major factor that limits the success and availability of allogeneic bone marrow or stem cell transplantation (collective referred to herein as allo-BMT) for treating some forms of otherwise incurable hematological malignancies, such as leukemia. GVHD is a systemic inflammatory reaction which causes chronic illness and may lead to death of the host mammal. At present, allogeneic transplants invariably run a severe risk of associated GVHD, even where the donor has a high degree of histocompatibility with the host. GVHD is caused by donor T-cells reacting against systemically distributed incompatible host antigens, causing powerful inflammation. In GVHD, mature donor T-cells that recognize differences between donor and host become systemically activated.

In certain aspects, the compounds and compositions as described herein can be used to treat patients suffering from sinusoidal obstruction syndrome (SOS) or complications associated therewith. SOS, also known as hepatic venoocclusive disease, was first diagnosed in cases of liver disease caused by the ingestion of herbal teas or food sources containing pyrrolizidine alkaloids from Crotalaria, Heliotropium and Senecio or from the consumption of bread made from inadequately winnowed wheat contaminated by seeds from these plants. With the modern development of chemotherapy, cases of SOS developed from the long-term use of azathioprine for immunosuppression after renal and liver transplantation and from the use of chemotherapeutic agents. Liver complications of chemotherapy are seen most commonly after high dose chemotherapy, with or without total body irradiation, or high dose radiation to the liver. Liver toxicity is not an uncommon side effect of high-dose chemotherapy. Liver toxicity also occurs after chemotherapy and/or liver irradiation when there is no bone marrow transplantation and hence, conditioning regimens used for marrow ablation are the most common cause of SOS. SOS is a common complication of chemotherapy with gemtuzumab ozogamicin or actinomycin D, or after long-term immunosuppression with azathioprine in kidney or liver transplantation patients. Other chemotherapeutic agents associated with liver toxicity and SOS include dacarbazine, cytosine arabinoside, mithrarnycin, 6-thioguanine, urethane, indicine N-oxide, alone and in combination. Milder forms of liver disease from chemotherapy which share the key aspect of sinusoidal endothelial cell injury include nodular regenerative hyperplasia, sinusoidal dilatation and peliosis hepatis. Combinations of irradiation and chemotherapy have also led to the development of SOS. For example, treating nephroblastoma (Wilms' tumor) with dactinomycin and abdominal irradiation has led to SOS.

Radiation-induced liver disease is a condition that shares some of the features of SOS, although there are differences in clinical presentation, histology and time course. Radiation-induced liver disease is seen with radiation doses in excess of 30 to 35 Gy in adults.

SOS has significant morbidity and mortality. The severity of SOS can be classified as mild (SOS is clinically obvious, but requires no treatment and resolves completely), moderate (SOS that causes signs and symptoms requiring treatment such as diuretics or pain medications, but resolves completely) or severe (SOS that requires treatment but that does not resolve before death or day 100. Some patients have subclinical liver damage, evinced by histologic evidence of liver toxicity in the absence of clinical signs and symptoms. Despite deep jaundice, patients with severe SOS seldom die of liver failure, but rather from renal and cardiopulmonary failure.

A clinically useful model for predicting the outcome of SOS after cyclophosphamide-based regimes is derived from rates of increase of both bilirubin and weight in the first two weeks following transplantation. Furthermore, a poor prognosis correlates with higher serum AST and ALT values, higher wedged hepatic venous pressure gradient, development of portal vein thrombosis, doubling of the baseline serum creatinine, and decreasing oxygen saturation. There is currently no prophylactic treatment for either SOS or radiation-induced liver disease, and there are no proven therapeutic remedies with high efficacy. The only therapeutic modality with some proven efficacy is the combination of heparin plus tissue plasminogen activator. However, this combination can only be safely used in a very limited group of patients and has efficacy in less than 30% of this limited population of patients.

SOS is the dose-limiting toxicity for several chemotherapeutic drugs and limits patient eligibility. A prophylactic treatment of SOS would have a significant impact on the ability to use high dose chemotherapy. Development of therapies to treat SOS after onset of the disease would be of value in unexpected cases of chemotherapy-induced liver disease.

The molecular events have been best characterized in the rat monocrotaline model. Monocrotaline, the pyrrolizidine alkaloid found in Crotalaria, is one of the best-studied toxins involving SOS. The monocrotaline model of SOS has the same histologic characteristics as the human disease, as well as the same “clinical features,” i.e., hyperbilirubinemia, hepatomegaly, and ascites formation. In this model, the first morphologic change noted by electron microscopy is loss of the sinusoidal endothelial cell fenestration and the appearance of gaps in the sinusoidal endothelial cell barrier. Studies with in vivo microscopy and confirmation by electron microscopy have shown that sinusoidal endothelial cells round up, and red blood cells begin to penetrate into the space of Disse beneath the rounded up endothelial cells and dissect off the sinusoidallining. The sloughed sinusoidal lining cells (i.e., Kupffer cells, sinusoidal endothelial cells, and stellate cells) embolize downstream and obstruct sinusoidal flow. By the time hepatocyte necrosis is observed, there is extensive denudation of the sinusoidal lining. Early on, there is loss of Kupffer cells, but there is a significant influx of monocytes within the sinusoids, which exacerbates the obstruction of sinusoidal flow by the embolized sinusoidal lining cells. The rounding up or swelling of sinusoidal endothelial cells is the initiating event in SOS and leads to dissection of the sinusoidal lining, which embolizes and blocks the microcirculation.

In certain aspects, the compounds and compositions as described herein can be used to treat patients suffering from cancers of the blood and complications associated therewith. Such cancer group includes hematological malignancies. Examples of cancers of the blood include acute myelogenous leukemia (AML), acute lymphoblastic leukemia (ALL), chronic myelogenous leukemia (CML) and multiple myeloma (MM).

Complications associated with a cancer of the blood include, for example, shortened life expectancy, organ damage, periodic or chronic pain, migration of cancer cells out of blood circulation, and reduction in red blood cells, white blood cells or platelets. It is desirable to prevent cancer cells from leaving the primary site, or to prevent extravasation of cancer cells from the bloodstream and infiltration into other tissues. Cancer cells while in the bloodstream are typically susceptible to chemotherapy, but are more difficult to treat once they leave the bloodstream. For example, cancer cells (such as MM cells) can extravasate from the bloodstream and infiltrate into bone marrow matrix where they are inaccessible to chemotherapeutic agents circulating in the bloodstream. Consequences of the complication of migration of cancer cells out of blood circulation include relapse (failure to cure) and disseminated disease (metastasis) leading, for example, to organ damage or failure. AML is an example of a blood cancer with the complication of migration of cancer cells out of blood circulation resulting in disseminated disease.

Acute myelogenous leukemia (also known as acute myeloid leukemia or AML) is a cancer of white blood cells, and in particular the myeloid line. It appears that AML arises from a single progenitor cell which has undergone genetic transformation to an abnormal cell with the ability to proliferate rapidly. These abnormal immature myeloid cells accumulate in the bone marrow. This accumulation in the bone marrow interferes with the production of normal blood cells, including a reduction in red blood cells, platelets and neutrophils. Eventually the bone marrow stops working correctly.

AML is one of the most common types of leukemia among adults, and the most common acute leukemia affecting adults. In the U.S. alone, there are approximately 12,000 new cases each year. The incidence of AML is expected to increase as the population ages. In addition, in the U.S., about 11% of the cases of leukemia in childhood are AML. Chemotherapy is generally used to treat AML. Only a minority of patients are cured with current therapy.

Chemotherapy has a number of deleterious side effects. One of the side effects is myeloablative bone marrow toxicities. Bone marrow is the tissue that fills the inside of some bones. Examples of such bones are sternum, hip, femur and humerus. Bone marrow contains stem cells that develop into several types of blood cells: erythrocytes (red blood cells), leukocytes (white blood cells) and thrombocytes (platelets). Cells in the bone marrow are susceptible to the effects of chemotherapy due to their rapid rate of division. Bone marrow is prevented by chemotherapeutic agents from forming new blood cells. With time after exposure to a chemotherapeutic agent, counts of the blood cells will fall at various rates, depending upon the particular type of cell as their average life spans differ. Low white blood cell count, for example, makes an individual more susceptible to infection. Low red blood cell count, for example, causes an individual to be fatigued. Low platelet count, for example, impairs an individual's ability to make a blood clot.

In certain aspects, the compounds and compositions as described herein can be used to treat patients suffering from epilepsy. Epilepsy is one of the most common neurological problems, with up to about 1% of the population afflicted. Epileptogenesis is a term used to refer to the process of a normal brain becoming epileptic in the first place. In the process (which may occur after acute brain injury), lesions and changes in the brain progress to the formation of chronic seizures. Acute injury to the brain can arise, for example, from traumatic physical brain injury (i.e., closed head injury), stroke or infection. The term epileptogenesis is also used for the process of how a mildly epileptic brain can worsen. While the reduction or prevention of seizures has understandably been the focus of substantial medical research, one ultimately would like to prevent epilepsy or stop its progression by the development of an anti-epileptogenic agent.

The term “epilepsy” as commonly used includes more than one type of disorder, and in its generic meaning is better termed “epileptic syndromes.” An example of an epileptic syndrome is Rasmussen's syndrome.

Rasmussen's syndrome was first described in 1958 and remains an unresolved medical problem. This devastating disorder afflicts mainly children and can destroy a cerebral hemisphere. Progressive neurological deterioration (including brain atrophy) and seizures are associated with Rasmussen's syndrome. Medical treatment has typically included anticonvulsant therapy and hemispherectomy surgery where half of the brain is removed. The surgery has been more effective than anti-seizure drugs in stopping the seizures. However, side effects of the surgery typically include the addition of a limp to walking and running, and on the side opposite to the surgery there is significant impairment of hand function and loss of fine motor skills.

In some embodiments, a compound of Formula (I) and/or a pharmaceutical composition comprising at least one compound of Formula (I) may be used for treating at least one of the diseases, disorders, and conditions described herein or for the preparation or manufacture of a medicament for use in treating at least one of the diseases, disorders, and/or conditions described herein. Each of these methods and uses is described in greater detail.

As understood by a person of ordinary skill in the medical art, the terms, “treat” and “treatment,” include medical management of a disease, disorder, or condition of a subject (i.e., patient, individual) (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment. Subjects in need of treatment include those who already have the disease, condition, or disorder as well as subjects prone to have or at risk of developing the disease, condition, or disorder, and those in which the disease, condition, or disorder is to be prevented (i.e., decreasing the likelihood of occurrence of the disease, disorder, or condition).

In some embodiments of the methods described herein, the subject is a human. In some embodiments of the methods described herein, the subject is a non-human animal. A subject in need of treatment as described herein may exhibit at least one symptom or sequelae of the disease, disorder, or condition described herein or may be at risk of developing the disease, disorder, or condition. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing a disease, disorder, or condition described herein can readily be determined by a person of ordinary skill in the medical and clinical arts. Determining and adjusting an appropriate dosing regimen (e.g., adjusting the amount of compound per dose and/or number of doses and frequency of dosing) can also readily be performed by a person of ordinary skill in the medical and clinical arts. One or any combination of diagnostic methods, including physical examination, assessment and monitoring of clinical symptoms, and performance of analytical tests and methods described herein, may be used for monitoring the health status of the subject.

Also provided herein are pharmaceutical compositions comprising at least one compound of Formula (I). In some embodiments, the pharmaceutical composition further comprises at least one additional pharmaceutically acceptable ingredient.

In pharmaceutical dosage forms, any one or more of the compounds of the present disclosure may be administered in the form of a pharmaceutically acceptable derivative, such as a salt, and/or it/they may also be used alone and/or in appropriate association, as well as in combination, with other pharmaceutically active compounds.

An effective amount or therapeutically effective amount refers to an amount of a compound of the present disclosure or a composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject.

In general, the amount of at least one compound of Formula (I) as described herein, that is present in a dose, may range from about 0.01 μg to about 100 mg per kg weight of the subject. In some embodiments, the amount of at least one compound of Formula (I) that is present in a dose may range from about 0.01 μg to about 40 mg per kg weight of the subject. In some embodiments, the amount of at least one compound of Formula (I) that is present in a dose may range from about 0.01 μg to about 20 mg per kg weight of the subject. In some embodiments, the amount of at least one compound of Formula (I) that is present in a dose may range from about 0.1 mg to about 100 mg per kg weight of the subject. In some embodiments, the amount of at least one compound of Formula (I) that is present in a dose may range from about 0.1 mg to about 40 mg per kg weight of the subject. In some embodiments, the amount of at least one compound of Formula (I) that is present in a dose may range from about 0.1 mg to about 20 mg per kg weight of the subject.

The minimum dose that is sufficient to provide effective therapy may be used in some embodiments. Subjects may generally be monitored for therapeutic effectiveness using assays suitable for the disease or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease or disorder may be determined according to parameters understood by a person of ordinary skill in the medical art.

Pharmaceutical compositions may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical composition described herein may be sterile aqueous or sterile non-aqueous solutions, suspensions or emulsions, and may additionally comprise at least one pharmaceutically acceptable excipient (i.e., a non-toxic material that does not interfere with the activity of the active ingredient). Such compositions may be in the form of a solid, liquid, or gas (aerosol). Alternatively, the compositions described herein may be formulated as a lyophilizate, or compounds described herein may be encapsulated within liposomes using technology known in the art. The pharmaceutical compositions may further comprise at least one additional pharmaceutical acceptable ingredient, which may be biologically active or inactive. Non-limiting examples of such ingredients include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in pharmaceutical compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient is selected based on the mode of administration, as well as the chemical composition of the active ingredient(s). Pharmaceutical compositions may be formulated for the particular mode of administration. For parenteral administration, pharmaceutical compositions may further comprise water, saline, alcohols, fats, waxes, and buffers. For oral administration, pharmaceutical compositions may further comprise at least one ingredient chosen, for example, from any of the aforementioned excipients, solid excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid pharmaceutical composition may include, for example, at least one the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition an injectable pharmaceutical composition, and in some embodiments, the injectable pharmaceutical composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneräs et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound or composition comprising the same.

### EXAMPLES

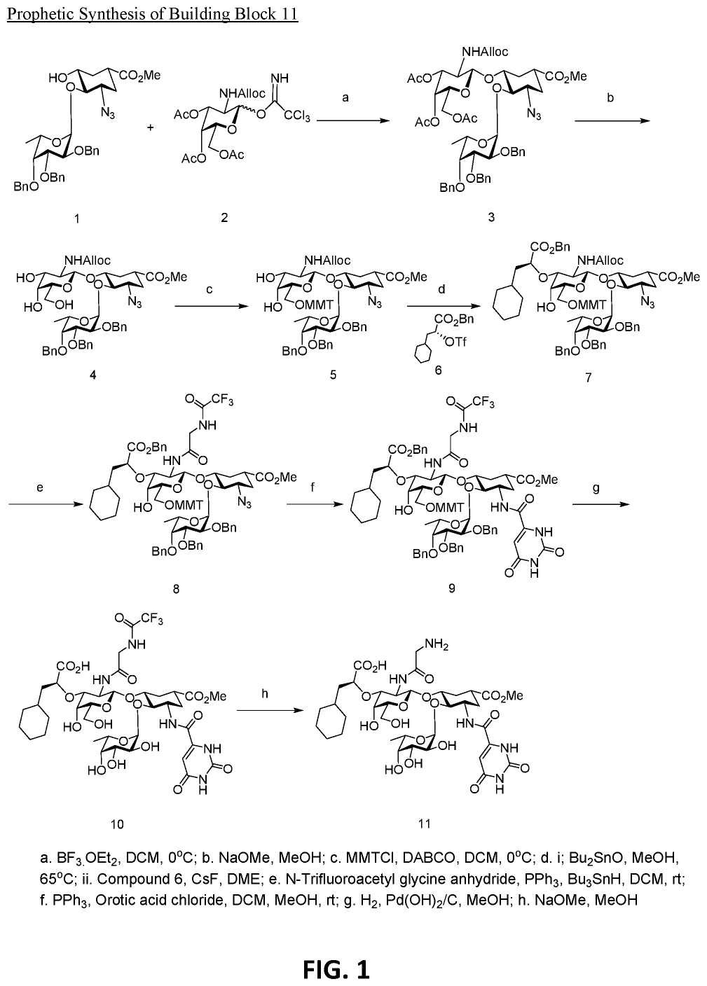

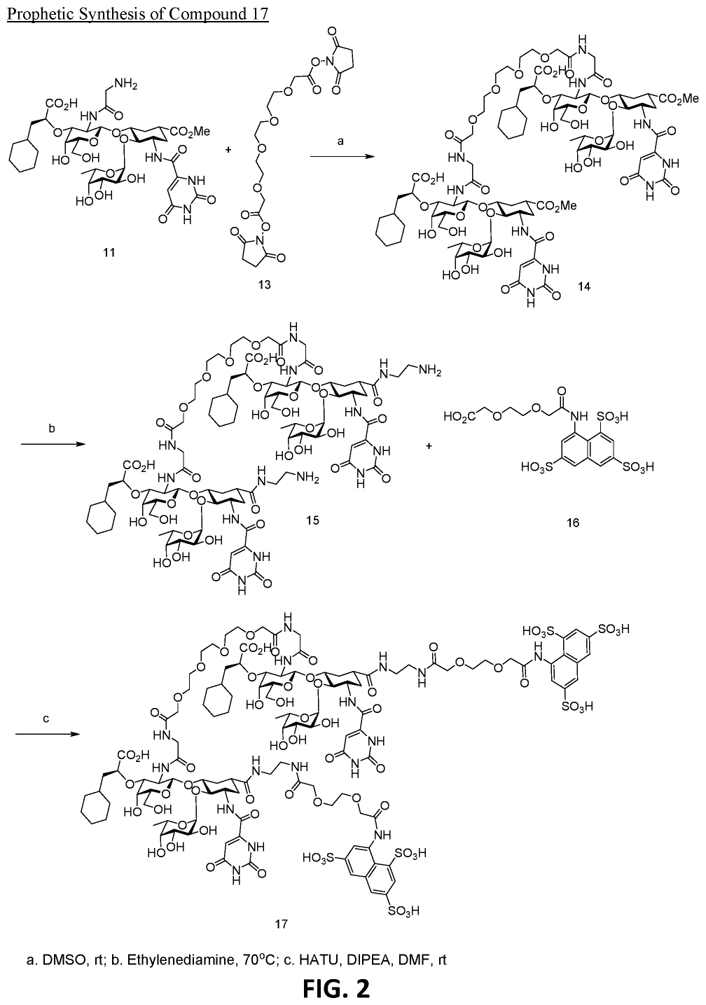

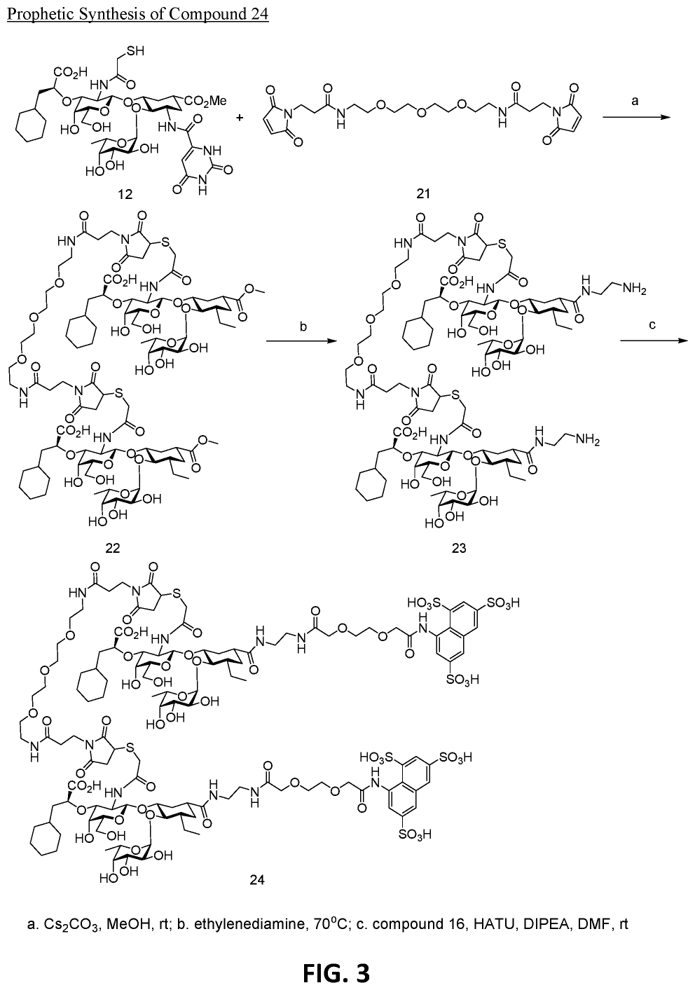

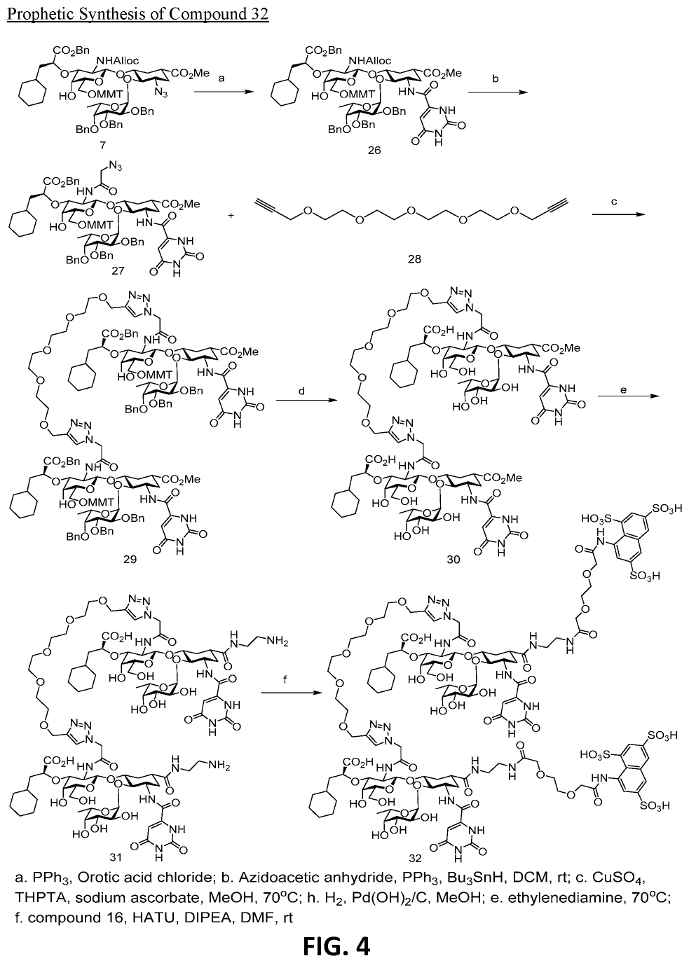

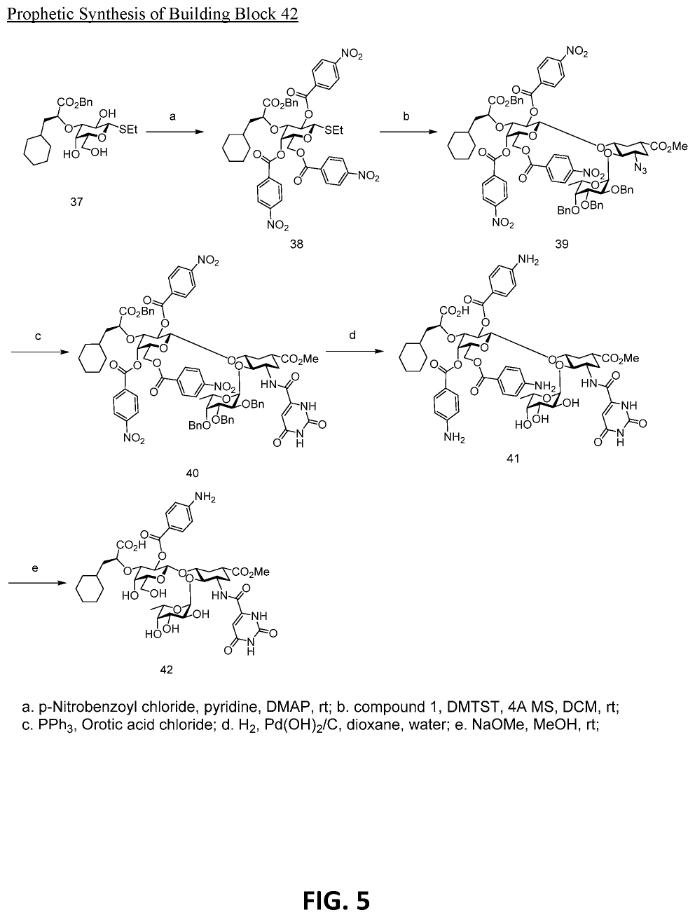

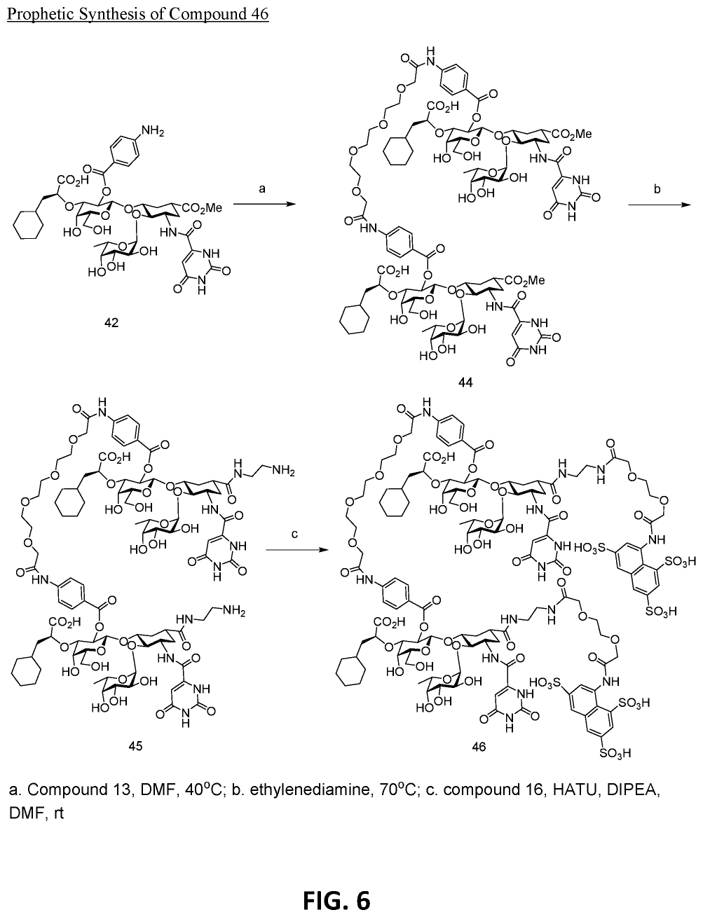

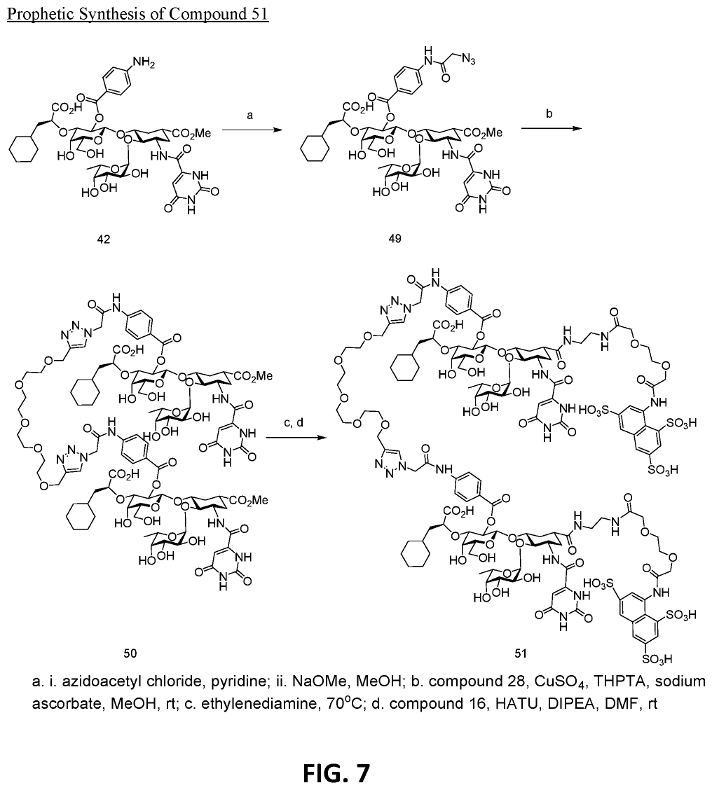

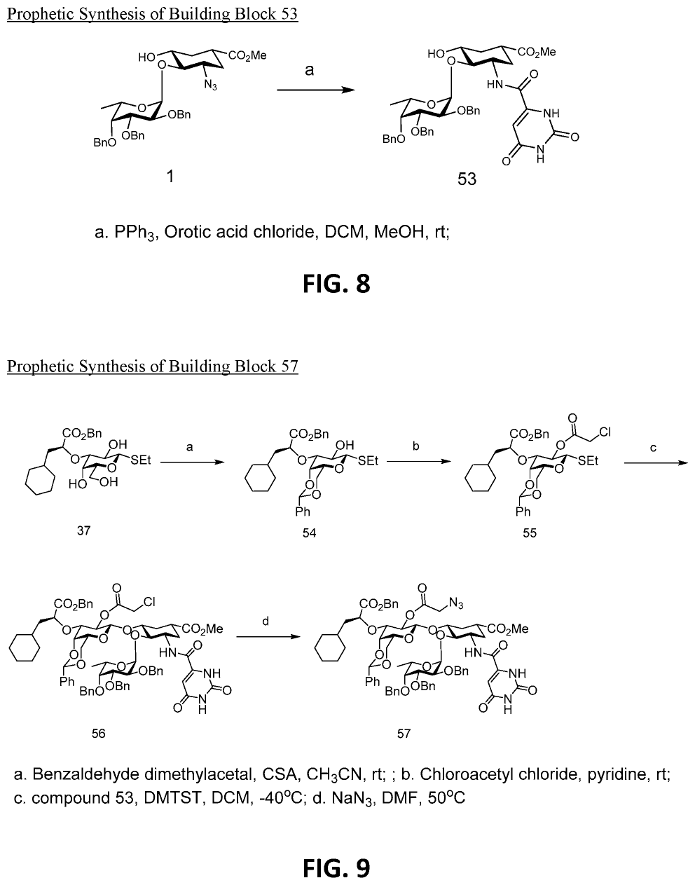

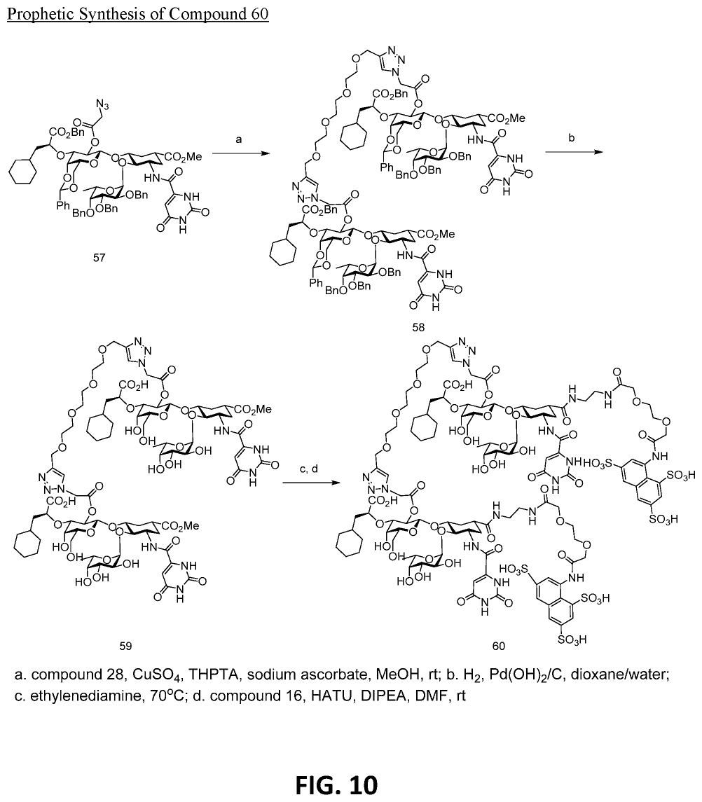

Compounds of Formula (I) may be prepared as shown in FIGS. 1-10. It is understood that one of ordinary skill in the art may be able to make these compounds by similar methods or by combining other methods known to one of ordinary skill in the art. It is also understood that one of ordinary skill in the art would be able to make other compounds of Formula (I) not specifically illustrated herein by using appropriate starting components and modifying the parameters of the Prophetic Synthesis as needed. In general, starting components may be obtained from sources such as Sigma Aldrich, Lancaster Synthesis, Inc., Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include but are not limited to t-butoxycarbonyl, benzyloxycarbonyl, and the like. Suitable protecting groups for mercapto include but are not limited to C(O)R″ (where R″ is alkyl, aryl or arylalkyl), p-methoxybenzyl, trityl and the like. Suitable protecting groups for carboxylic acid include but are not limited to alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Analogous reactants to those described herein may be identified through the indices of known chemicals prepared by the Chemical Abstract Service of the American Chemical Society, which are available in most public and university libraries, as well as through on line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the Prophetic Synthesis of reactants useful in the preparation of compounds of the present disclosure, or provide references to articles that describe the preparation, include for example, “Synthetic Organic Chemistry,” John Wiley & Sons, Inc., New York; S. R. Sandier et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley Interscience, New York, 1992. Additional suitable reference books and treatise that detail the Prophetic Synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3 527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

### Example 1

### Prophetic Synthesis of Building Block Compound 11

Compound 3: A mixture of compound 1 (preparation described in WO 2007/028050) and compound 2 (preparation described in WO 2013/096926) (1.7 eq) is azeotroped three (3) times from toluene. The mixture is dissolved in DCM under argon and cooled on an ice bath. To this solution is added boron trifluoride etherate (1.5 eq). The reaction mixture is stirred twelve (12) hours at room temperature. The reaction is quenched by the addition of triethylamine (2 eq). The reaction mixture is transferred to a separatory funnel and washed one (1) time with half saturated sodium bicarbonate solution and one (1) time with water. The organic phase is dried over sodium sulfate, filtered, and concentrated. The residue is purified by flash chromatography to afford compound 3.

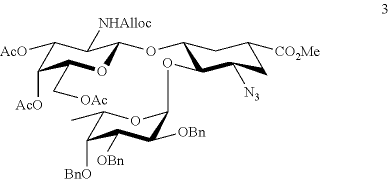

Compound 4: Compound 3 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed two (2) times with water. The organic phase is dried over magnesium sulfate, filtered, and concentrated. The residue is separated by flash chromatography to afford compound 4.

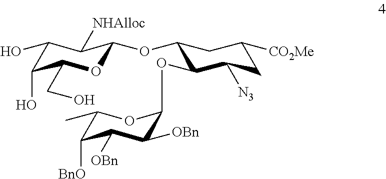

Compound 5: To a solution of compound 4 cooled on an ice bath is added DABCO (1.5 eq) followed by monomethyoxytrityl chloride (1.2 eq). The reaction mixture is stirred overnight allowing to warm to room temperature. The reaction mixture is concentrated and the residue is purified by flash chromatography to afford compound 5.

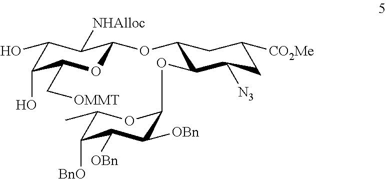

Compound 7: To a solution of compound 5 in methanol is added dibutyltin oxide (1.1 eq). The reaction mixture is refluxed for three (3) hours then concentrated. The residue is suspended in DME. To this suspension is added compound 6 (preparation described in Thoma et. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered, and concentrated. The residue is purified by flash chromatography to afford compound 7.

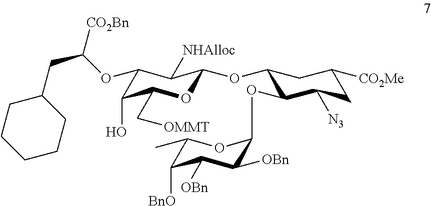

Compound 8: To a degassed solution of compound 7 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for twelve (12) hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 8.

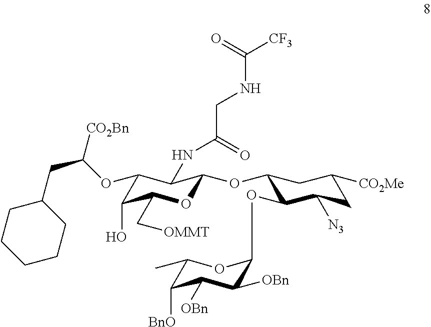

Compound 9: To a stirred solution of compound 8 in DCM/MeOH (25/1) at room temperature is added orotic acid chloride (5 eq) and triphenylphosphine (5 eq). The reaction mixture is stirred twenty-four (24) hours. The solvent is removed and the residue is separated by column chromatography to afford compound 9.

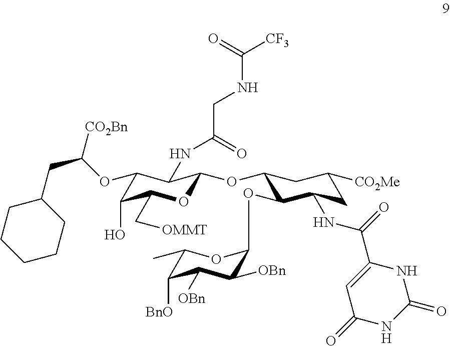

Compound 10: Compound 9 is dissolved in methanol and degassed. To this solution is added Pd(OH)₂/C. The reaction mixture is vigorously stirred under a hydrogen atmosphere for twelve (12) hours. The reaction mixture is filtered through a Celite pad. The filtrate is concentrated under reduced pressure to give compound 10.

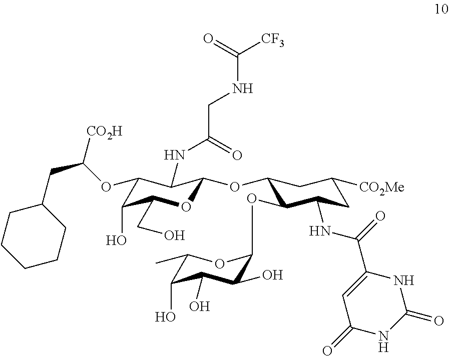

Compound 11: Compound 10 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 11.

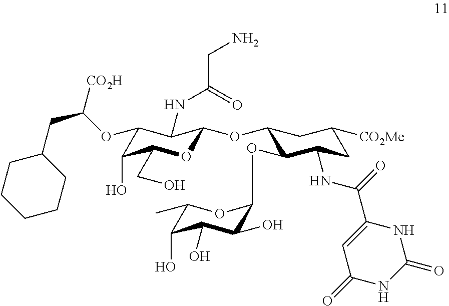

### Example 2

### Prophetic Synthesis of Building Block Compound 12

Compound 12: Compound 12 can be prepared according to the steps for the prophetic synthesis of compound 11 by substituting (acetylthio)acetyl chloride for N-trifluoroacetyl glycine anhydride in the preparation of compound 8.

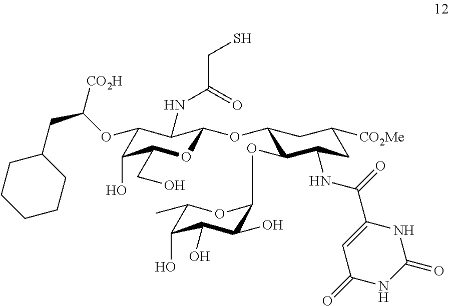

### Example 3

### Prophetic Synthesis of Multimeric Compound 17

Compound 14: A solution of compound 13 (0.4 eq) in DMSO is added to a solution of compound 11 (1 eq) and DIPEA (10 eq) in anhydrous DMSO at room temperature. The resulting solution is stirred overnight. The solution is dialyzed against distilled water for three (3) days with dialysis tube MWCO 1000 while distilled water is changed every twelve (12) hours. The solution in the tube is lyophilized to give compound 14.

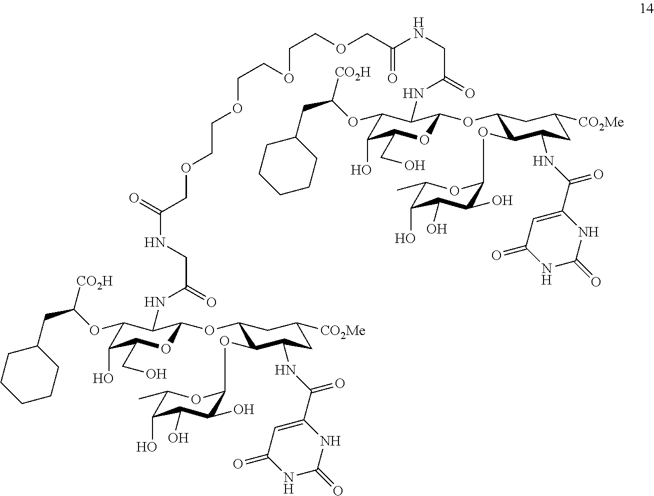

Compound 15: A solution of compound 14 in ethylenediamine is stirred overnight at 70° C. The reaction mixture is concentrated under reduced pressure and the residue is purified by reverse phase chromatography to give compound 15.

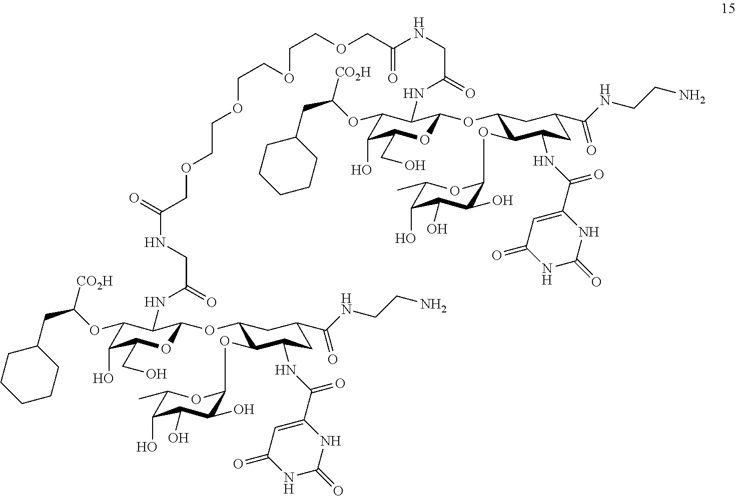

Compound 17: To a solution of compound 16 (2.2 eq) (preparation described in WO 2007/028050) in DMF at room temperature is added DIPEA (3 eq) and HATU (2.1 eq). The reaction mixture is stirred for 10 minutes at room temperature. A solution of compound 15 (1 eq) in DMF is added and the reaction mixture is stirred overnight at room temperature. The solvent is removed and the residue is separated by sephadex G-25 chromatography to afford compound 17.

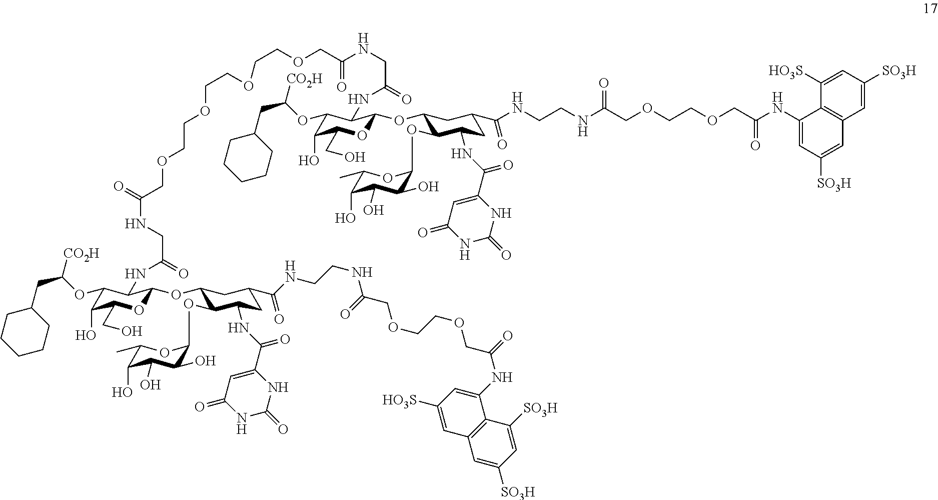

### Example 4

### Prophetic Synthesis of Multimeric Compound 18

Compound 18: Compound 18 can be prepared according to the prophetic synthesis of compound 17 by replacing compound 13 with PEG-11 diacetic acid di-NHS ester in the preparation of compound 14.

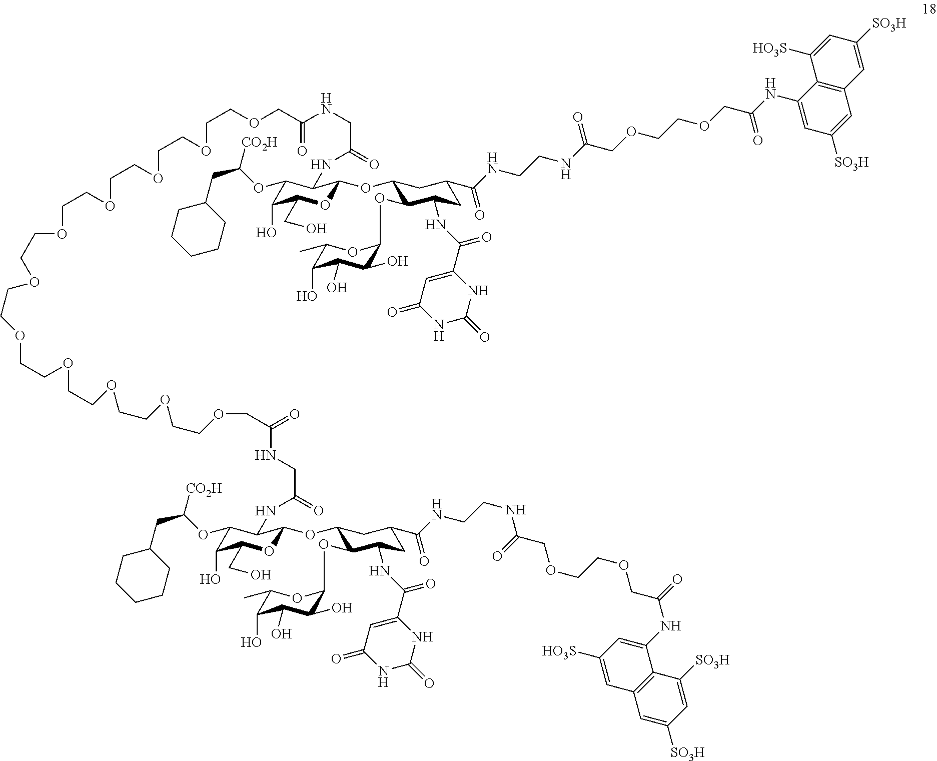

### Example 5

### Prophetic Synthesis of Multimeric Compound 19

Compound 19: Compound 19 can be prepared according to the prophetic synthesis of compound 17 by replacing compound 13 with PEG-15 diacetic acid di-NHS ester in the preparation of compound 14.

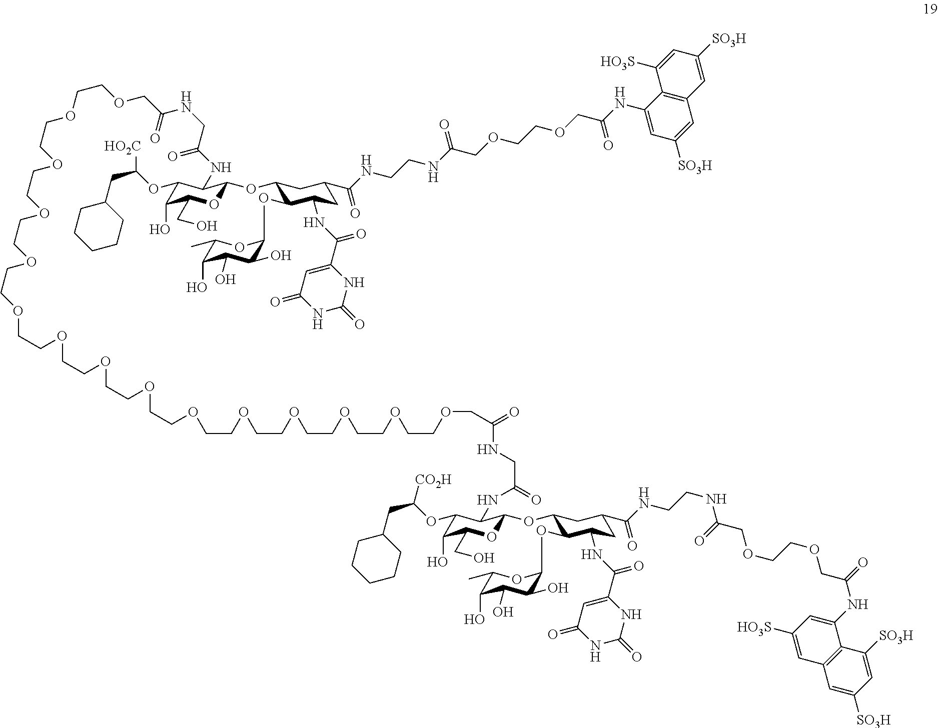

### Example 6

### Prophetic Synthesis of Multimeric Compound 20

Compound 20: Compound 20 can be prepared according to the prophetic synthesis of compound 17 by replacing compound 13 with ethylene glycol diacetic acid di-NHS ester in the preparation of compound 14.

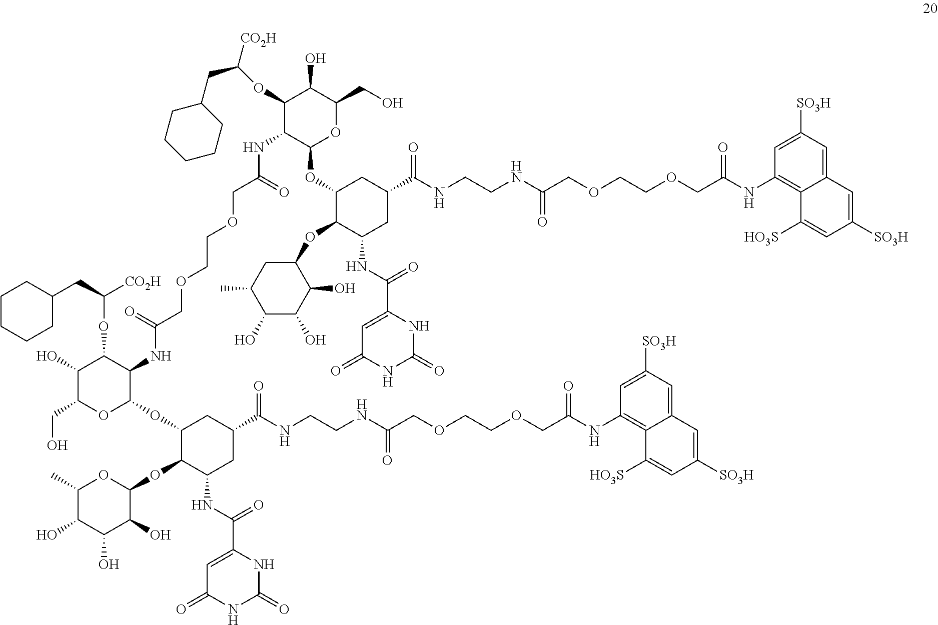 [SMILES: C[C@@H]1C[C@@H](O[C@@H]2[C@@H](NC(=O)c3cc(=O)[nH]c(=O)[nH]3)C[C@@H](C(=O)NCCNC(=O)COCCOCC(=O)Nc3cc(S(=O)(=O)O)cc4cc(S(=O)(=O)O)cc(S(=O)(=O)O)c34)C[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3C[C@H](C(=O)NCCNC(=O)COCCOCC(=O)Nc4cc(S(=O)(=O)O)cc5cc(S(=O)(=O)O)cc(S(=O)(=O)O)c45)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 7

### Prophetic Synthesis of Multimeric Compound 63

Compound 63: Compound 63 can be prepared according to the prophetic synthesis of compound 17 by replacing compound 13 with 3,3′-[[2,2-bis[[3-[(2,5-dioxo-1-pyrrolidinyl)oxy]-3-oxopropoxy]methyl]-1,3-propanediyl]bis(oxy)]bis-, 1,1′-bis(2,5-dioxo-1-pyrrolidinyl)-propanoic acid ester in the preparation of compound 14.

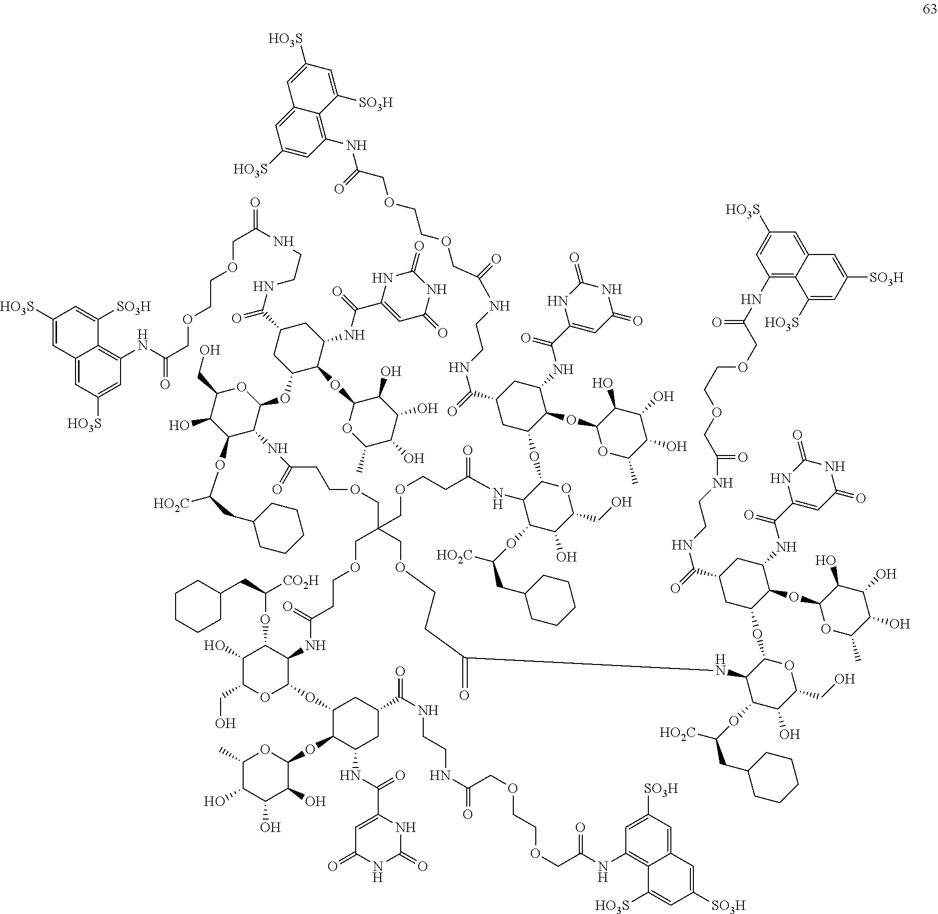

### Example 8

### Prophetic Synthesis of Multimeric Compound 24

Compound 22: To a solution of compound 12 in MeOH at room temperature is added compound 21 followed by cesium acetate (2.5 eq). The reaction mixture is stirred at room temperature until completion. The solvent is removed under reduced pressure. The product is purified by reverse phase chromatography to give compound 22.

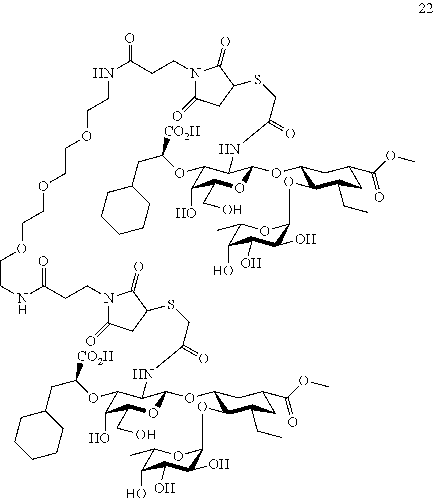

Compound 23: Compound 22 is dissolved in ethylenediamine and the reaction mixture is stirred overnight at 70° C. The reaction mixture is concentrated under reduced pressure and the residue is purified by reverse phase chromatography to give compound 23.

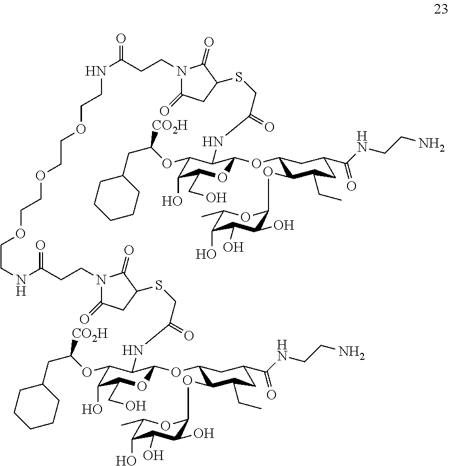

Compound 24: To a solution of compound 16 (synthesis described in WO 2007/028050) (2.2 eq) and DIPEA (2.5 eq) in DMF at room temperature is added HATU (2.3 eq). The solution is stirred for twenty (20) min. This solution is slowly added to a solution of compound 23 (1 eq) in DMF at room temperature. The resulting solution is stirred twelve (12) hrs. The solvent is removed and the residue is separated by sephadex G-25 chromatography to afford compound 24.

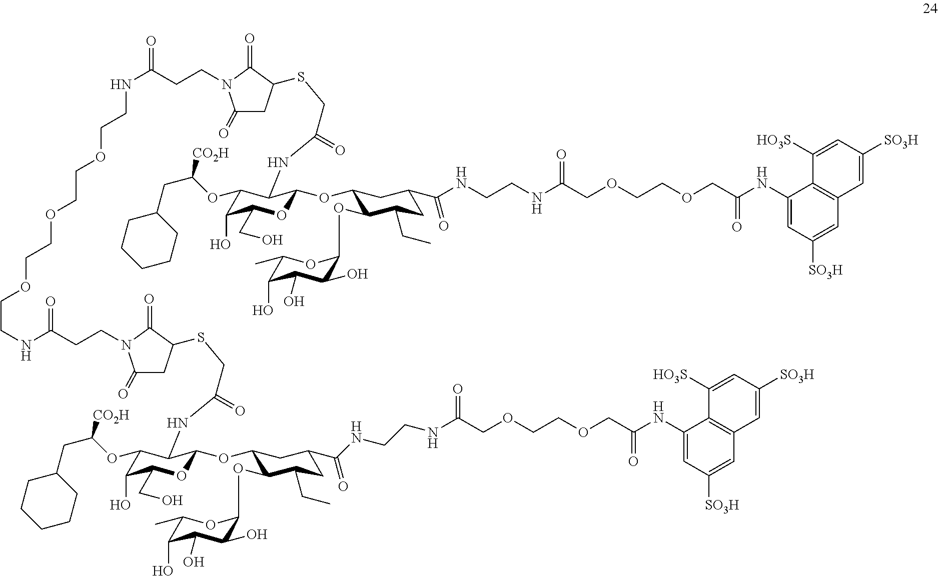

### Example 9

### Prophetic Synthesis of Multimeric Compound 25

Compound 25: Compound 25 can be prepared according to the prophetic synthesis of compound 24 by substituting PEG-6-bis maleimidoylpropionamide for compound 21.

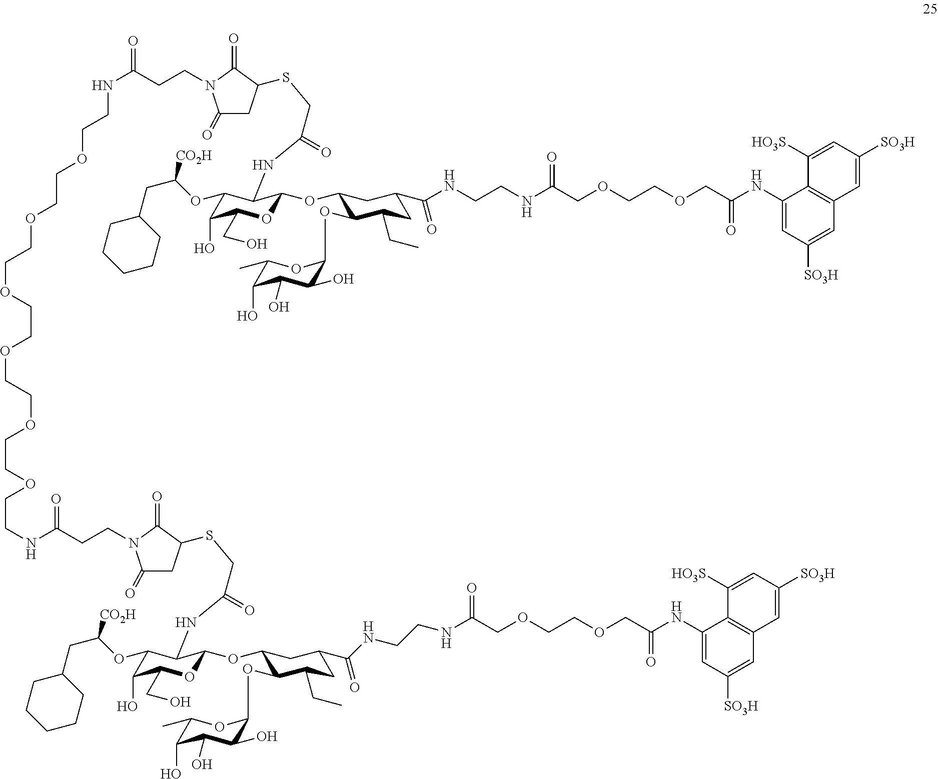

### Example 10

### Synthesis of Multimeric Compound 64

Compound 64: Compound 64 can be prepared according to the prophetic synthesis of compound 24 substituting compound 21 for, 1,1′-[[2,2-bis[[3-(2,5-dihydro-2, 5-dioxo-1H-pyrrol-1-yl)propoxy]methyl]-1,3-propanediyl]bis(oxy-3,1-propanediyl)]bis-1H-pyrrole-2,5-dione in the preparation of compound 22.

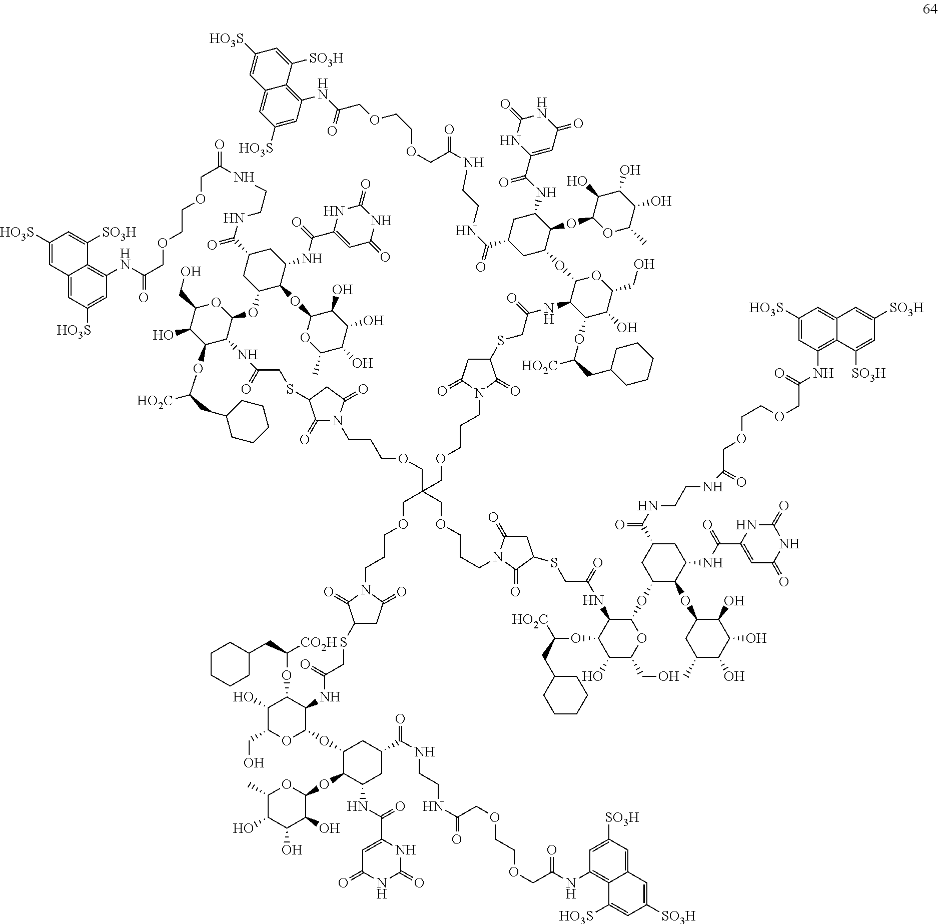

### Example 11

### Prophetic Synthesis of Multimeric Compound 32

Compound 26: To a stirred solution of compound 7 in DCM/MeOH (25/1) at room temperature is added orotic acid chloride (5 eq) and triphenylphosphine (5 eq). The reaction mixture is stirred twenty-four (24 hours). The solvent is removed and the residue is separated by column chromatography to afford compound 26.

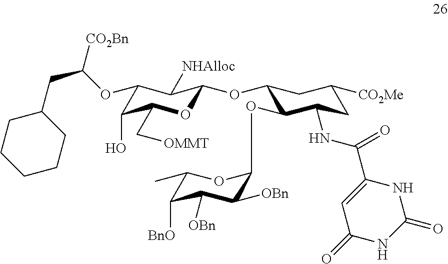

Compound 27: To a degassed solution of compound 26 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for twelve (12) hrs under a N₂ atmosphere at room temperature. The reaction mixture is diluted with DCM, washed with water, dried over Na₂SO₄, then concentrated. The crude product is purified by chromatography to give compound 27.

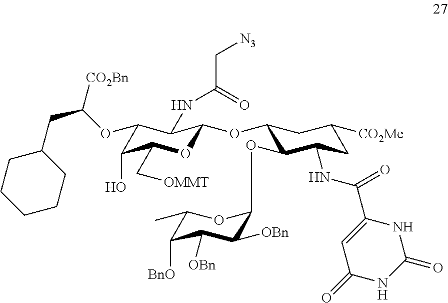

Compound 29: A solution of bispropagyl PEG-5 (compound 28) and compound 27 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred twelve (12) hrs at 70° C. The solution is cooled to room temperature and concentrated under reduced pressure. The crude product is purified by chromatography to give a compound 29.

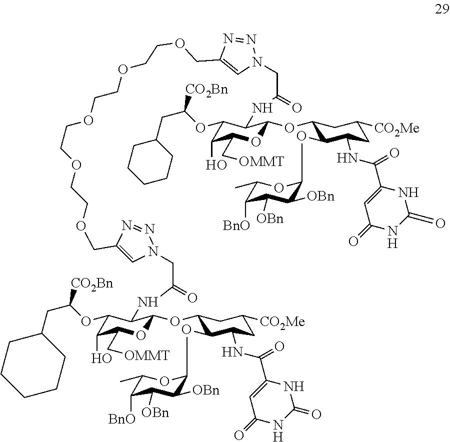

Compound 30: Compound 29 is dissolved in MeOH/i-PrOH (2/1) and hydrogenated in the presence of Pd(OH)₂ (0.2 g) at 1 atm of H₂ gas pressure for twenty-four (24) hrs at room temperature. The solution is filtered through a Celite pad. The filtrate is concentrated to give compound 30.

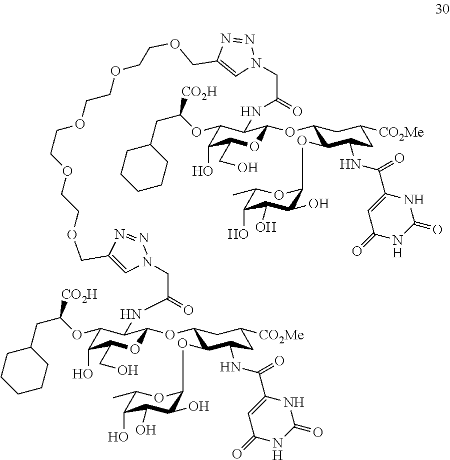

Compound 31: Compound 30 is dissolved in ethylenediamine and stirred for twelve (12) hrs at 70° C. The reaction mixture is concentrated under reduced pressure. The crude product is purified by C-18 column chromatography followed by lyophilization to give a compound 31.

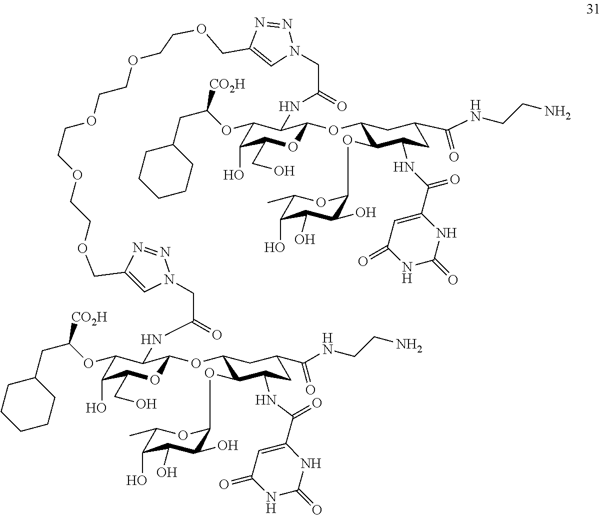

Compound 32: To a solution of compound 16 (synthesis described in WO 2007/028050) (2.2 eq) and DIPEA (2.5 eq) in DMF at room temperature is added HATU (2.3 eq). The solution is stirred for twenty (20) min. This solution is slowly added to a solution of compound 31 (1 eq) in DMF at room temperature. The resulting solution is stirred twelve (12) hrs. The reaction mixture is purified by sephadex G-25 chromatography to give compound 32.

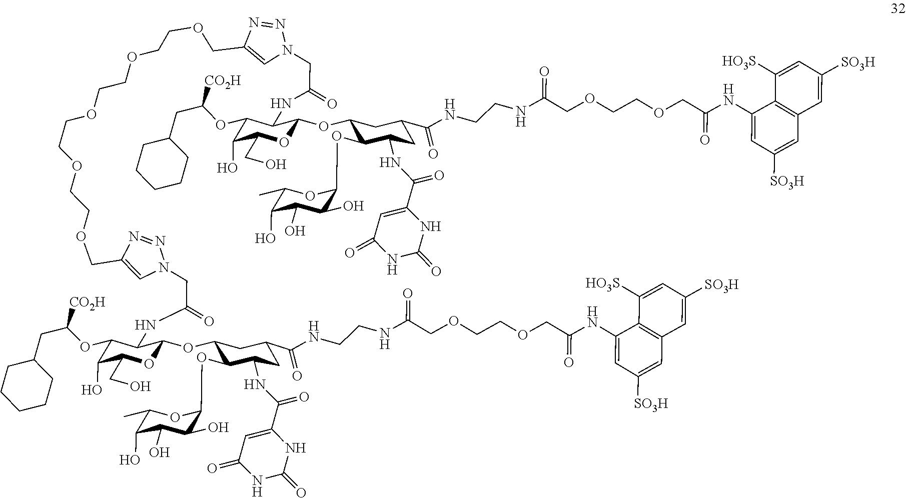

### Example 12

### Prophetic Synthesis of Multimeric Compound 33

Compound 33: Compound 33 can be prepared according to the prophetic synthesis of compound 32 using 3-azidopropanoic anhydride (Yang, C. et. al. JACS, (2013) 135(21), 7791-7794) in place of azidoacetic anhydride in the preparation of compound 27.

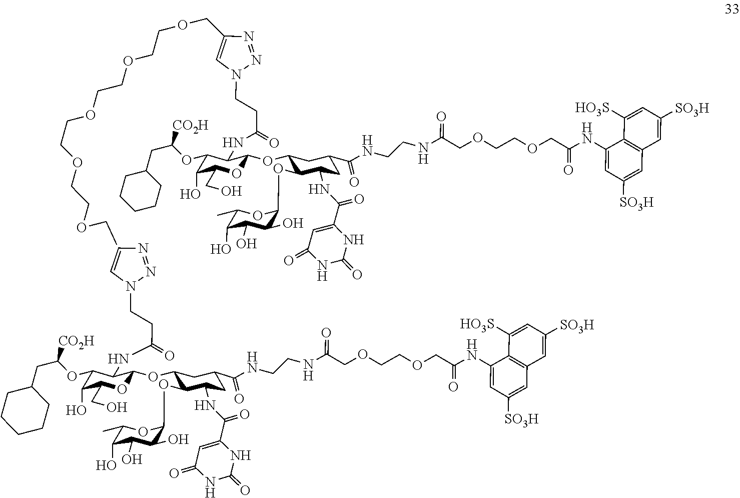

### Example 13

### Prophetic Synthesis of Multimeric Compound 34

Compound 34: Compound 34 can be prepared according to the prophetic synthesis of compound 32 using 3-azidobutanoic anhydride (Yang, C. et. al. JACS, (2013) 135(21), 7791-7794) in place of azidoacetic anhydride in the preparation of compound 27.

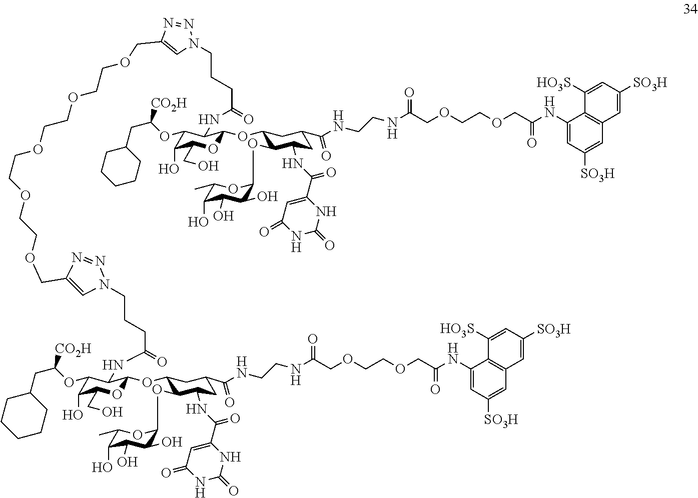

### Example 14

### Prophetic Synthesis of Multimeric Compound 35

Compound 35: Compound 35 can be prepared according to the prophetic synthesis of compound 32 using 4-azidobutanoic anhydride (Yang, C. et. al. JACS, (2013) 135(21), 7791-7794) in place of azidoacetic anhydride in step b and using 1,2-bis(2-propynyloxy) ethane in place of compound 28 in the preparation of compound 29.

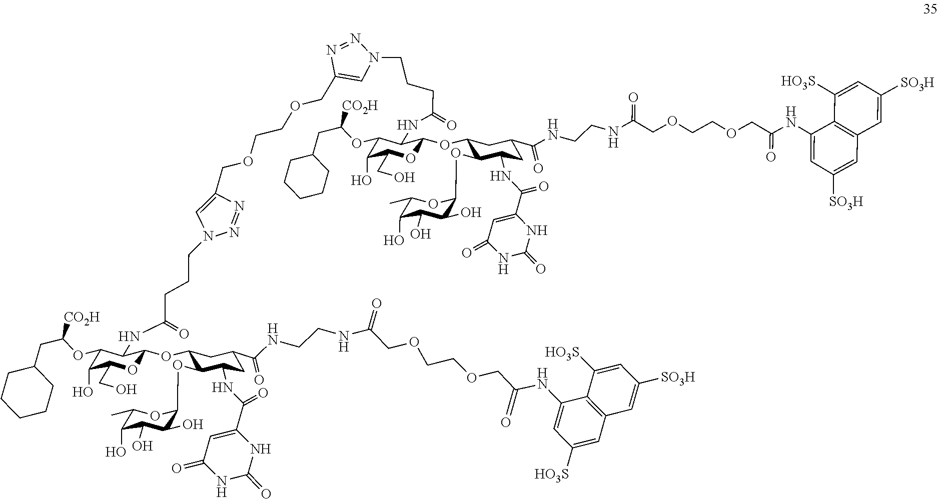

### Example 15

### Prophetic Synthesis of Multimeric Compound 36

Compound 36: Compound 36 can be prepared according to the prophetic synthesis of compound 32 using 4,7,10,13,16,19,22,25,28,31-decaoxatetratriaconta-1, 33-diyne in place of compound 28 in the preparation of compound 29.

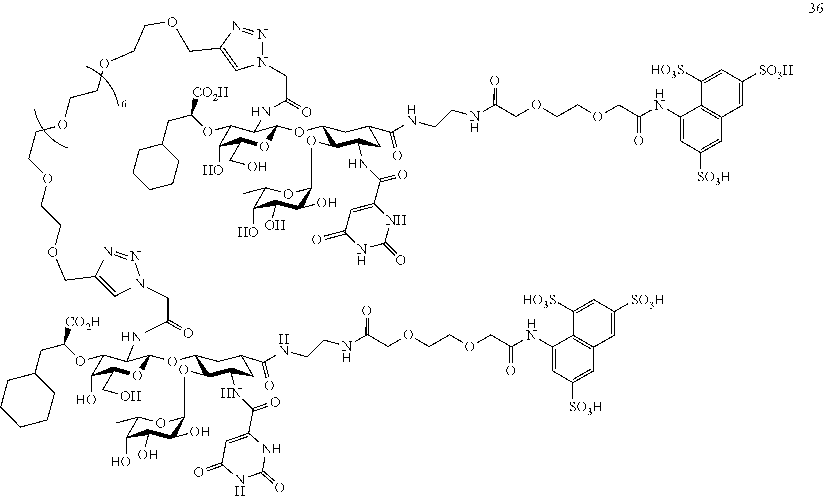

### Example 16

### Prophetic Synthesis of Multimeric Compound 65

Compound 65: Compound 65 can be prepared according to the prophetic synthesis of compound 32 using 3,3′-[[2,2-bis[(2-propyn-1-yloxy)methyl]-1,3-propanediyl]bis(oxy)]bis-1-propyne in place of compound 28 in the preparation of compound 29.

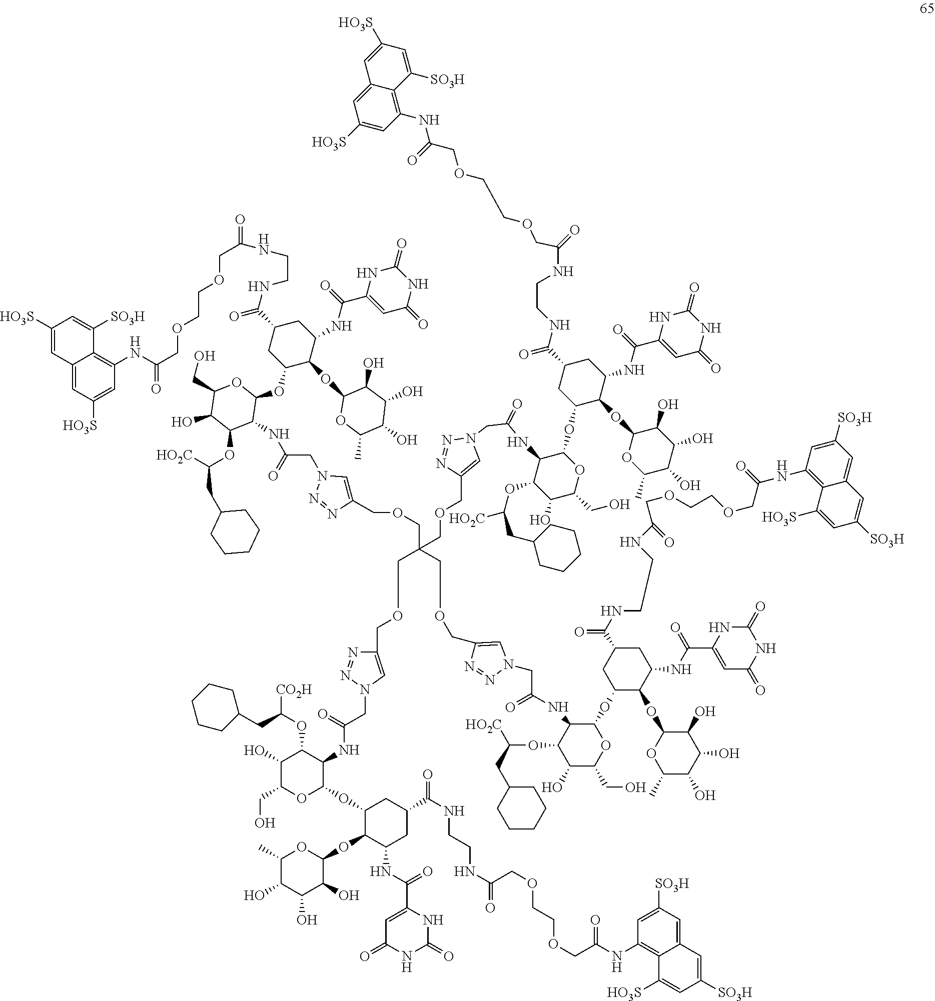

### Example 17

Compound 66: Compound 66 can be prepared according to the prophetic synthesis of compound 32 using 3,3′-[oxybis[[2,2-bis[(2-propyn-1-yloxy)methyl]-3,1-propanediyl]oxy]]bis-1-propyne in place of compound 28 in the preparation of compound 29.

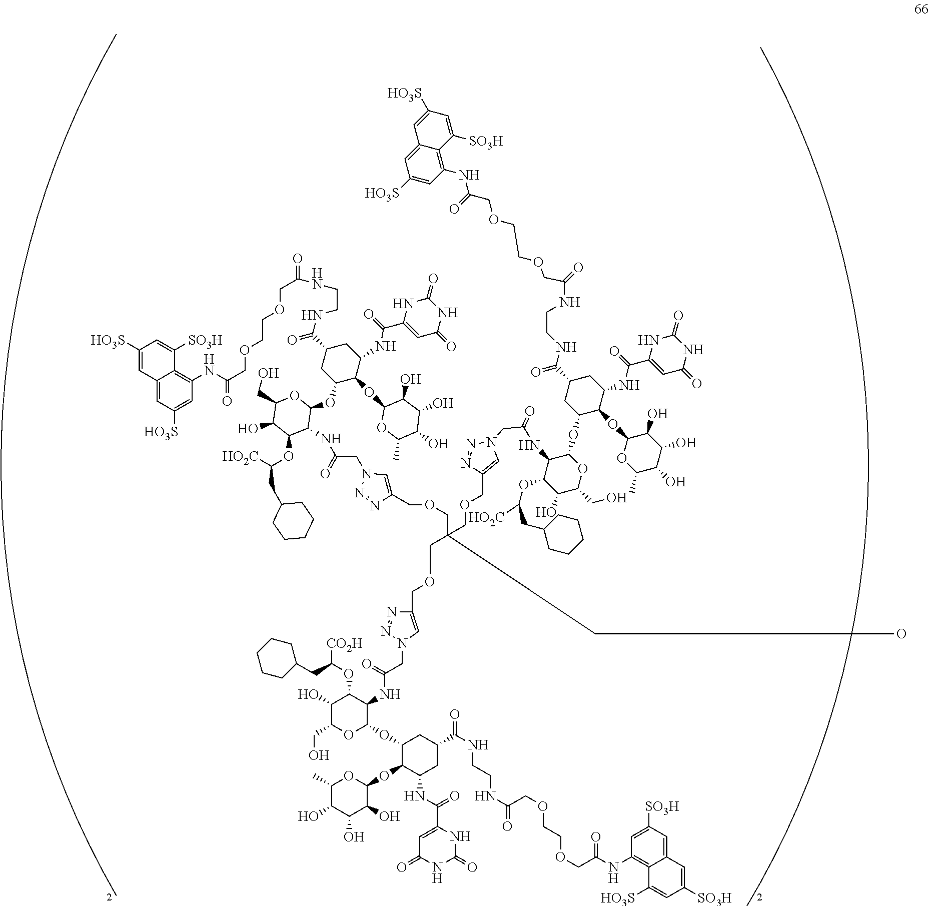

### Example 18

### Prophetic Synthesis of Building Block Compound 42

Compound 38: Compound 37 (prepared according to Banteli et. al. Helvetica Chimica Acta, 83, 2000, 2893) is dissolved in pyridine at room temperature. A catalytic amount of DMAP is added followed by p-nitrobenzoyl chloride (10 eq). The reaction mixture is stirred overnight. The solvent is removed under reduced pressure. The residue is dissolved in ethyl acetate, transferred to a separatory funnel and washed two times with 0.1N HCl, two times with saturated sodium bicarbonate solution, and one time with water. The organic phase is dried over sodium sulfate, filtered, then concentrated. The residue is separated by flash chromatography to afford compound 38.

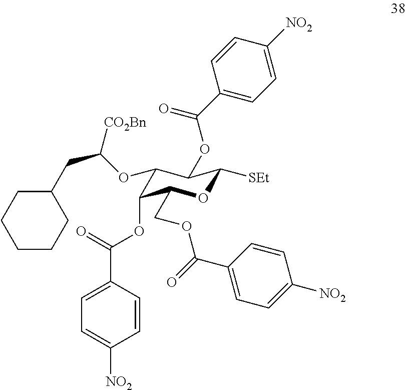

Compound 39: Activated powdered 4 Å molecular sieves are added to a solution of compound 38 and compound 1 (2 eq) in dry DCM under argon. The mixture is stirred for 2 hours at room temperature. Solid DMTST (1.5 eq) is added in 4 portions over 1.5 hours. The reaction mixture is stirred overnight at room temperature. The reaction mixture is filtered through Celite, transferred to a separatory funnel and washed two times with half saturated sodium bicarbonate and two times with water. The organic phase is dried over sodium sulfate, filtered and concentrated. The residue is separated by flash chromatography to afford compound 39.

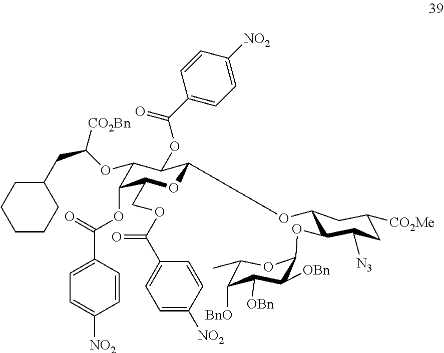

Compound 40: Compound 39 and orotic acid chloride (1.2 eq) are dissolved in DCM at room temperature. Triphenylphosphine (1.2 eq) is added and the reaction mixture is stirred overnight at room temperature. The solvent is removed and the residue separated by column chromatography to afford compound 40.

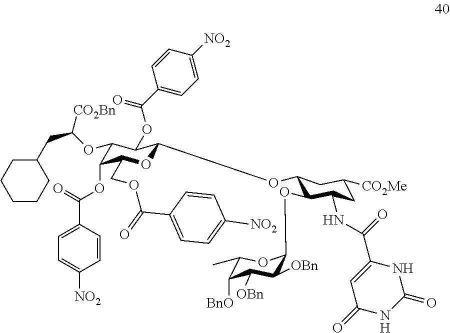

Compound 41: To a solution of compound 40 in dioxane/water (4/1) is added Pd(OH)₂/C. The reaction mixture is stirred vigorously overnight under a hydrogen atmosphere. The reaction mixture is filtered through Celite and concentrated. The residue is purified by column chromatography to afford compound 41.

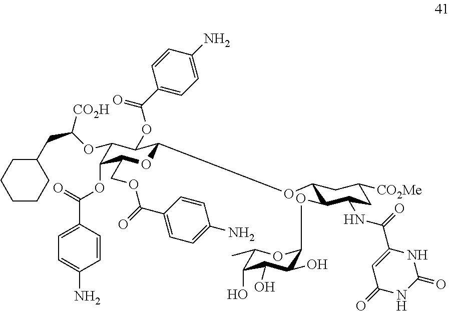

Compound 42: Compound 41 is dissolved in methanol at room temperature. Sodium methoxide (1.2 eq) is added and the reaction mixture is stirred overnight at room temperature. Acetic acid (1.5 eq) is added and the reaction mixture is concentrated. The residue is separated by column chromatography to afford compound 42.

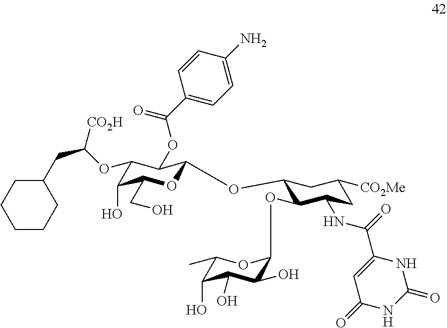

### Example 19

### Prophetic Synthesis of Building Block Compound 43

Compound 43: Using the sequence outlined in the Prophetic Synthesis of Compound 42, compound 43 can be prepared substituting m-nitrobenzoyl chloride for p-nitrobenzoyl chloride in the preparation of compound 38.

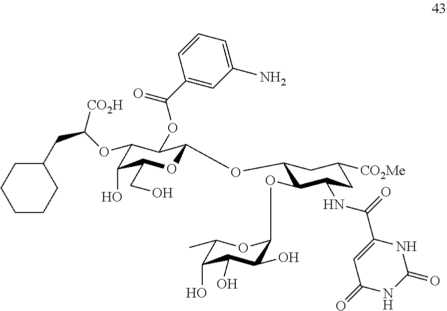

### Example 20

### Prophetic Synthesis of Multimeric Compound 46

Compound 44: Compound 42 (2 eq) and compound 13 are dissolved in DMF and stirred overnight at 40° C. The solvent is removed and the residue is purified by C-18 reverse phase chromatography to afford compound 44.

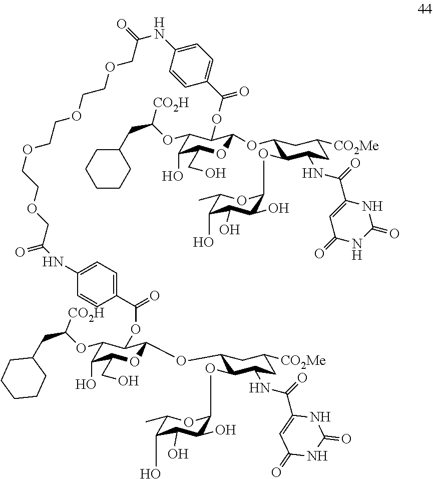

Compound 45: Compound 44 is dissolved in ethylenediamine and stirred at 70° C. overnight. The reaction mixture is concentrated and the residue is separated by sephadex G-25 chromatography to afford compound 45.

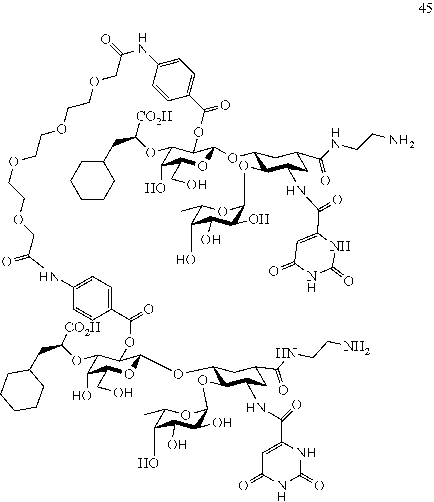

Compound 46: To a solution of compound 16 (synthesis described in WO 2007/028050) (2.2 eq) and DIPEA (2.5 eq) in DMF at room temperature is added HATU (2.3 eq). The solution is stirred for twenty (20) min. This solution is slowly added to a solution of compound 45 (1 eq) in DMF at room temperature. The resulting solution is stirred twelve (12) hrs. The reaction mixture is purified by sephadex G-25 chromatography to give compound 46.

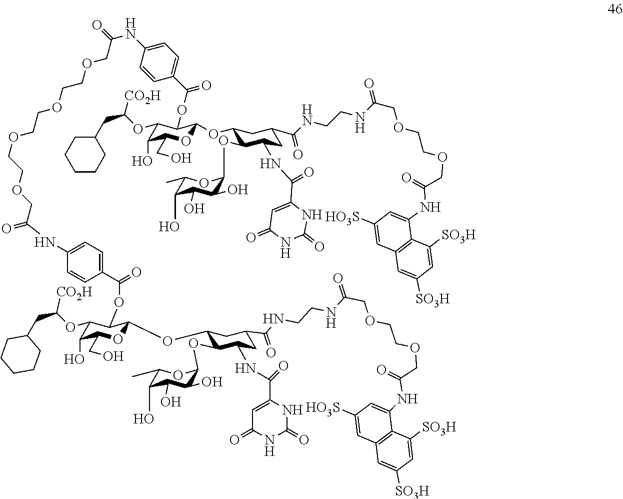

### Example 21

### Prophetic Synthesis of Multimeric Compound 47

Compound 47: Compound 47 can be prepared according to the prophetic synthesis of compound 46 by replacing compound 13 with PEG-8 diacetic acid di-NHS ester in the preparation of compound 45.

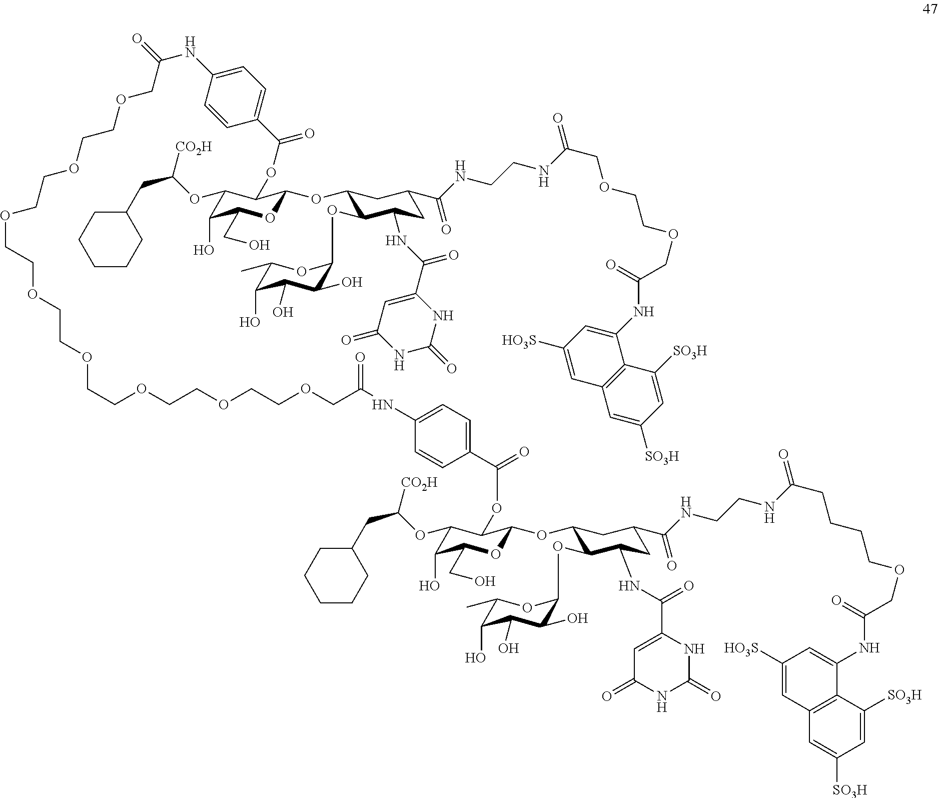

### Example 22

### Prophetic Synthesis of Multimeric Compound 48

Compound 48: Compound 48 can be prepared according to the prophetic synthesis of compound 46 by replacing compound 42 with compound 43 in the preparation of compound 45.

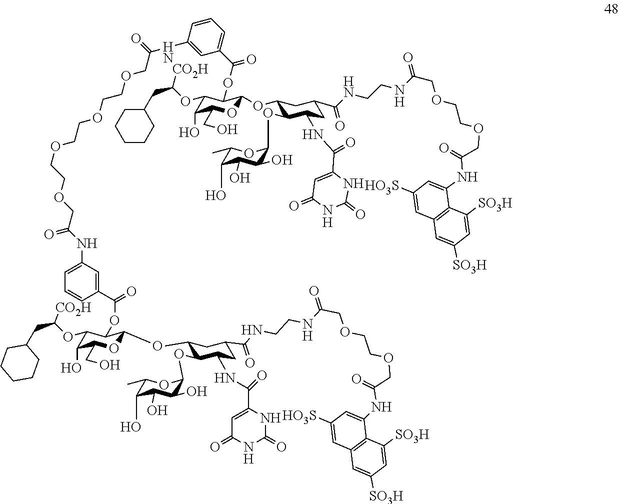

### Example 23

### Prophetic Synthesis of Multimeric Compound 51

Compound 49: Compound 42 is dissolved in pyridine and cooled on an ice bath. A catalytic amount of DMAP is added followed by azidoacetyl chloride (5 eq). The ice bath is removed and the reaction mixture is stirred at room temperature until completion. The solvent is removed under reduced pressure. The residue is dissolved in methanol at room temperature. Sodium methoxide (1.3 eq) is added and the reaction mixture is stirred until completion. The reaction is quenched by addition of acetic acid. The solvent is removed and the residue is separated by C-8 reverse phase chromatography to afford compound 49.

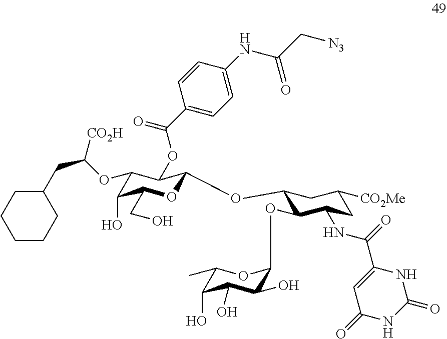

Compound 50: A solution of bipropargyl PEG- (compound 28) and compound 49 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at room temperature. The solution is concentrated under reduced pressure. The crude product is purified by chromatography to give a compound 50.

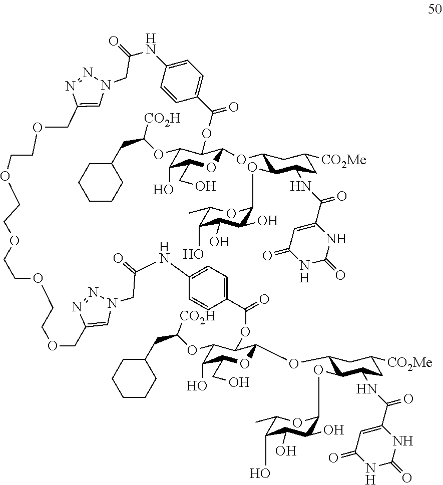

Compound 51: Compound 50 is dissolved in ethylenediamine and stirred for twelve (12) hrs at 70° C. The reaction mixture is concentrated under reduced pressure. The crude product is purified by C-18 column chromatography followed by lyophilization.

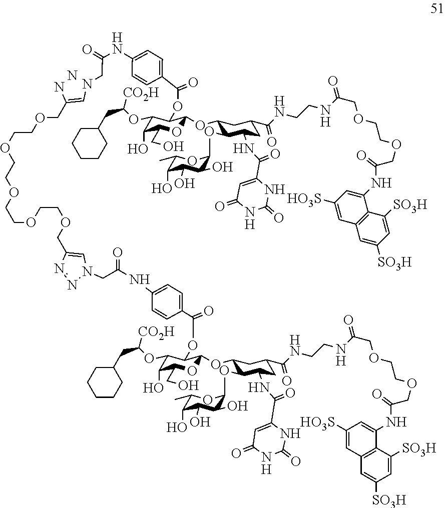

To a solution of compound 16 (synthesis described in WO 2007/028050) (2.2 eq) and DIPEA (2.5 eq) in DMF at room temperature is added HATU (2.3 eq). The solution is stirred for twenty (20) min. This solution is slowly added to a solution of the ethylenediamine adduct (1 eq) in DMF at room temperature. The resulting solution is stirred twelve (12) hrs. The reaction mixture is purified by sephadex G-25 chromatography to give compound 51.

### Example 24

### Prophetic Synthesis of Multimeric Compound 52

Compound 52: Compound 52 can be prepared according to the prophetic synthesis of compound 51 by replacing compound 42 with compound 43 in the preparation of compound 49.

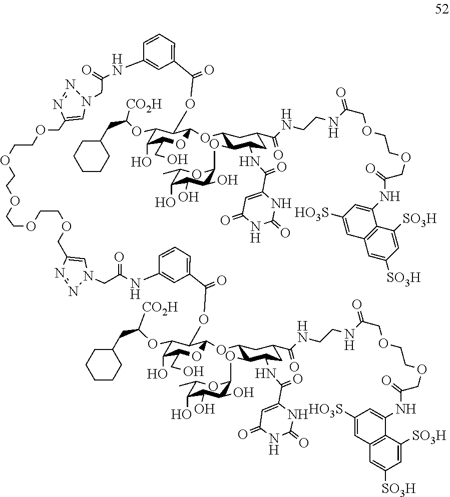

### Example 25

### Prophetic Synthesis of Compound 67

Compound 67: Compound 67 can be prepared according to the prophetic synthesis of compound 51 using 3,3′-[[2,2-bis[(2-propyn-1-yloxy)methyl]-1,3-propanediyl]bis(oxy)]bis-1-propyne in place of compound 28 in the preparation of compound 50.

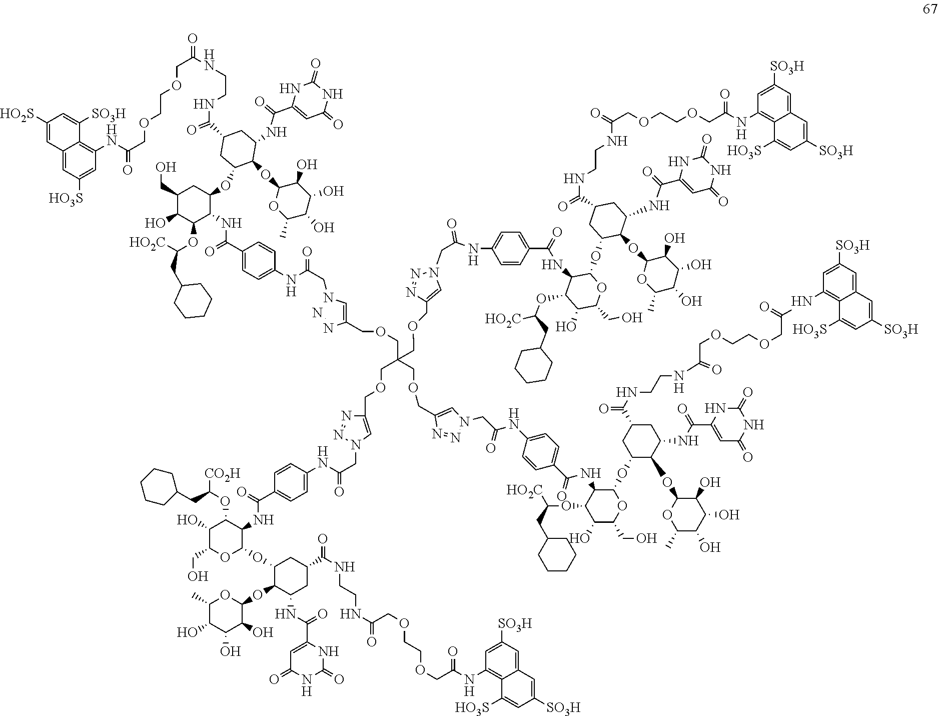

### Example 26

### Prophetic Synthesis of Multimeric Compound 68

Compound 68: Compound 68 can be prepared according to the prophetic synthesis of compound 51 using compound 43 in place of compound 42 in step a and using 3, 3′-[[2,2-bis[(2-propyn-1-yloxy)methyl]-1,3-propanediyl]bis(oxy)]bis-1-propyne in place of compound 28 in the preparation of compound 50.

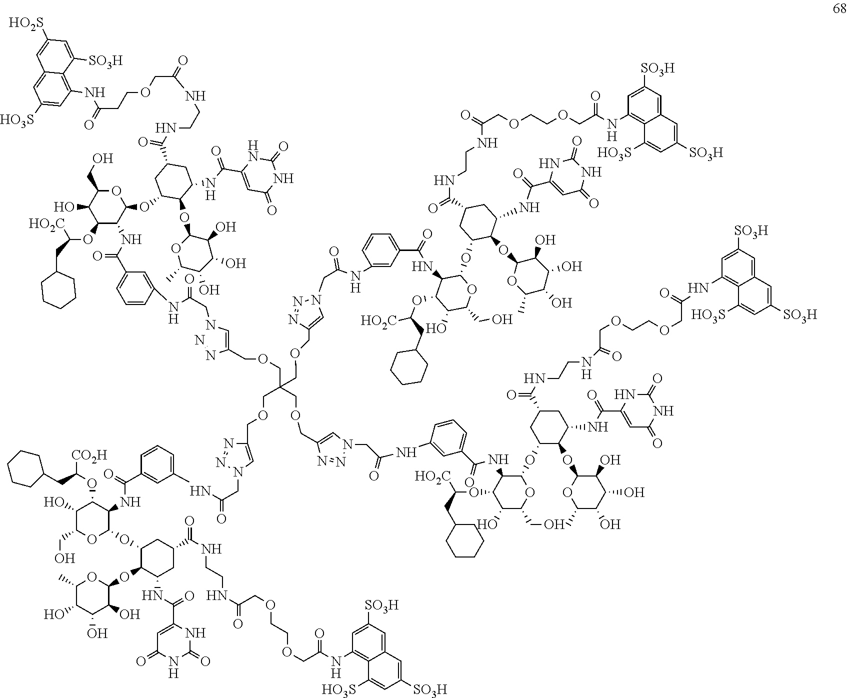

### Example 27

### Prophetic Synthesis of Building Block Compound 53

Compound 53: To a stirred solution of compound 1 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 53.

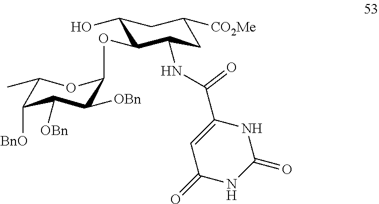

### Example 28

### Prophetic Synthesis of Building Block Compound 57

Compound 54: Compound 37 is dissolved in acetonitrile at room temperature. Benzaldehyde dimethylacetal (1.1 eq) is added followed by camphorsulfonic acid (0.2 eq). The reaction mixture is stirred until completion. Triethylamine is added. The solvent is removed and the residue separated by flash chromatography to afford compound 54.

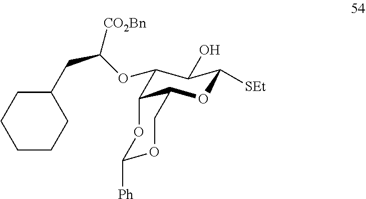

Compound 55: Compound 54 is dissolved in pyridine at room temperature. Dimethylaminopyridine (0.01 eq) is added followed by chloroacetyl chloride (2 eq). The reaction mixture is stirred until completion. The solvent is removed under educed pressure. The residue is dissolved in ethyl acetate, transferred to a separatory funnel and washed two times with 0.1N HCl and two times with water. The organic phase is dried over sodium sulfate, filtered, and concentrated. The residue is separated by column chromatograph to afford compound 55.

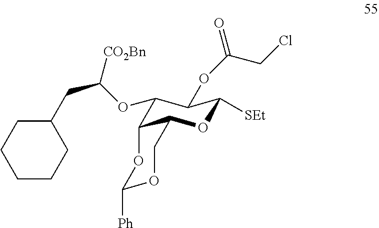

Compound 56: Activated powdered 4 Å molecular sieves are added to a solution of compound 55 and compound 53 (2 eq) in dry DCM under argon. The mixture is stirred for 2 hours at room temperature. Solid DMTST (1.5 eq) is added in four portions over one and one-half (1.5) hours. The reaction mixture is stirred overnight at room temperature. The reaction mixture is filtered through Celite, transferred to a separatory funnel and washed two times with half saturated sodium bicarbonate and two times with water. The organic phase is dried over sodium sulfate, filtered and concentrated. The residue is separated by flash chromatography to afford compound 56.

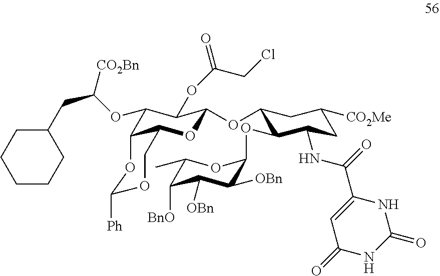

Compound 57: Compound 56 is dissolved in DMF. Sodium azide (1.5 eq) is added and the reaction mixture is stirred at 50° C. until completion. The reaction mixture is cooled to room temperature, diluted with ethyl acetate and transferred to a separatory funnel. The organic phase is washed four (4) times with water then dried over sodium sulfate and concentrated. The residue is separated by column chromatography to afford compound 57.

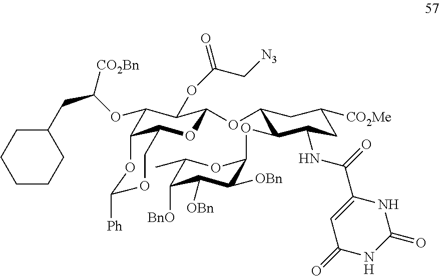

### Example 29

### Prophetic Synthesis of Multimeric Compound 60

Compound 58: A solution of bispropagyl PEG-5 (compound 28) and compound 57 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred twelve (12) hrs at 50° C. The solution is concentrated under reduced pressure. The crude product is purified by chromatography to give a compound 58.

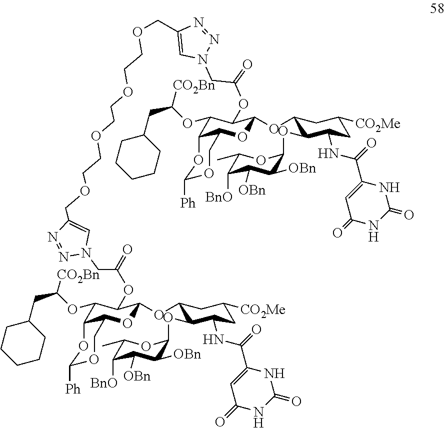

Compound 59: To a solution of compound 58 in dioxane/water (4/1) is added Pd(OH)₂/C. The reaction mixture is stirred vigorously overnight under a hydrogen atmosphere. The reaction mixture is filtered through Celite and concentrated. The residue is purified by C-19 reverse phase column chromatography to afford compound 59.

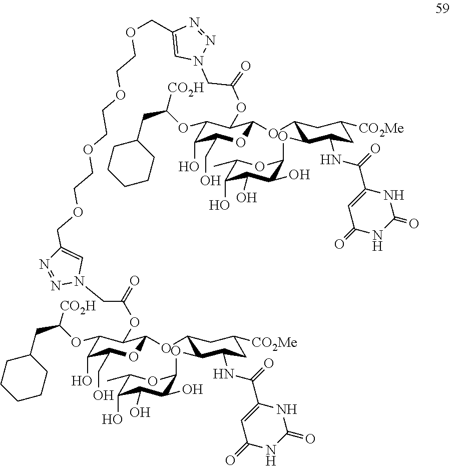

Compound 60: Compound 59 is dissolved in ethylenediamine and stirred for twelve (12) hrs at 70° C. The reaction mixture is concentrated under reduced pressure. The crude product is purified by C-8 column chromatography followed by lyophilization.

To a solution of compound 16 (synthesis described in WO 2007/028050) (2.2 eq) and DIPEA (2.5 eq) in DMF at room temperature is added HATU (2.3 eq). The solution is stirred for twenty (20) min. This solution is slowly added to a solution of the ethylenediamine adduct (1 eq) in DMF at room temperature. The resulting solution is stirred twelve (12) hrs. The reaction mixture is purified by sephadex G-25 chromatography to give compound 60.

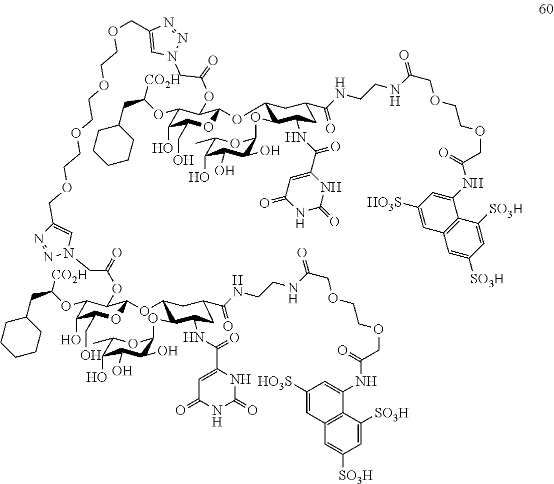

### Example 30

### Prophetic Synthesis of Multimeric Compound 61

Compound 61: Compound 61 can be prepared according to the prophetic synthesis of compound 60 by replacing compound 28 with PEG-8 bis propargyl ether in the preparation of compound 58.

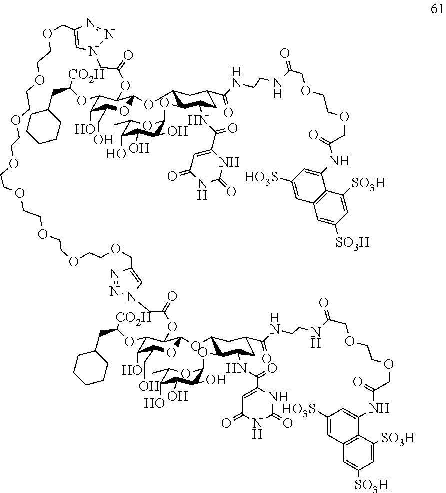

### Example 31

### Prophetic Synthesis of Multimeric Compound 62

Compound 62: Compound 62 can be prepared according to the prophetic synthesis of compound 60 by replacing compound 28 with ethylene glycol bis propargyl ether in the preparation of compound 58.

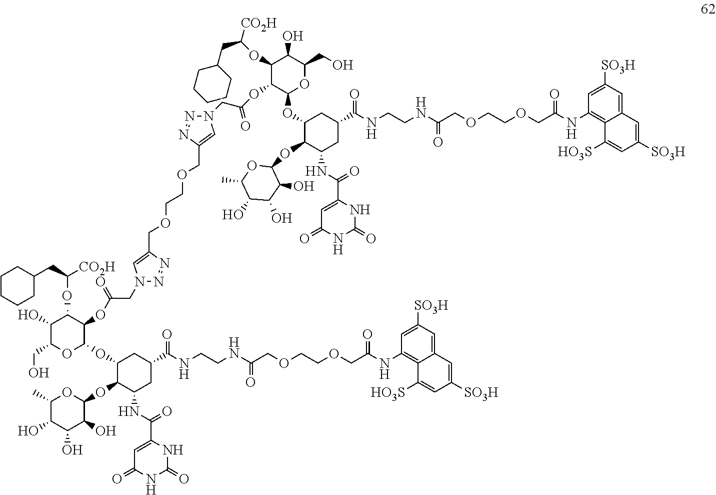

### Example 32

### Prophetic Synthesis of Multimeric Compound 69

Compound 69: Compound 69 can be prepared according to the prophetic synthesis of compound 60 using 3,3′-[[2,2-bis[(2-propyn-1-yloxy)methyl]-1,3-propanediyl]bis(oxy)]bis-1-propyne in place of compound 28 in the preparation of compound 58.

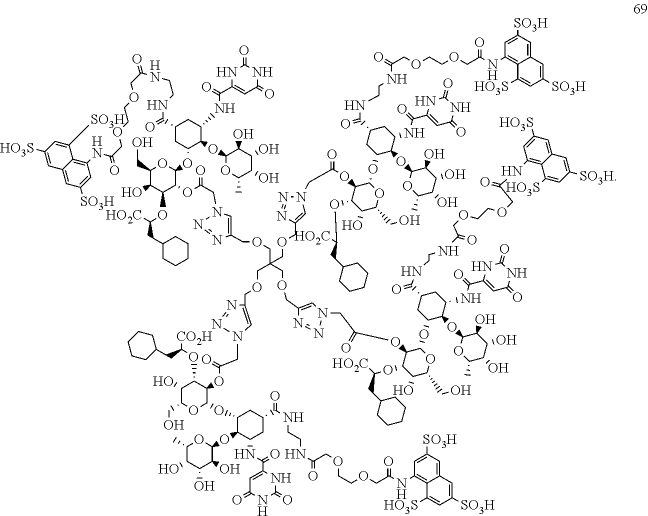

### Example 33

### Affinity Assay for E-Selectin Antagonist

The E-selectin antagonist activity may be determined using the assay described in example 21 of WO 2007/028050.

### Example 34

### Affinity Assay for P-Selectin Antagonists

The P-selectin antagonist activity may be determined using the assay described in example 22 of WO 2007/028050.

The various embodiments described above can be combined to provide further embodiments. All U.S. patents, U.S. patent application publications, U.S. patent applications, non-U.S. patents, non-U.S. patent applications, and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments

What is claimed is:

1. At least one compound chosen from multimeric selectin-modulators of Formula (I): 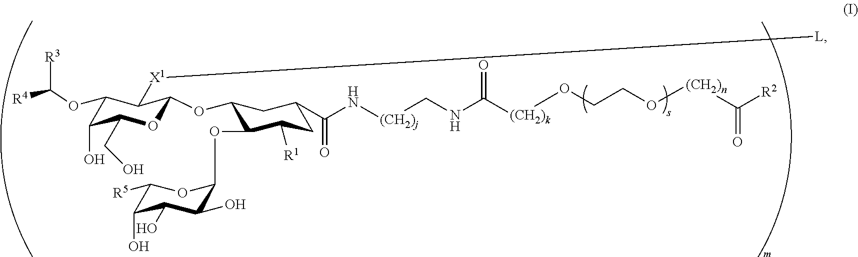
prodrugs thereof, and pharmaceutically acceptable salts of any of the foregoing, wherein
each R¹, which may be identical or different, is independently chosen from H, C₁₋₈ alkyl, C₂₋₈ alkenyl, C₂₋₈ alkynyl, C₄₋₁₆ cycloalkylalkyl, 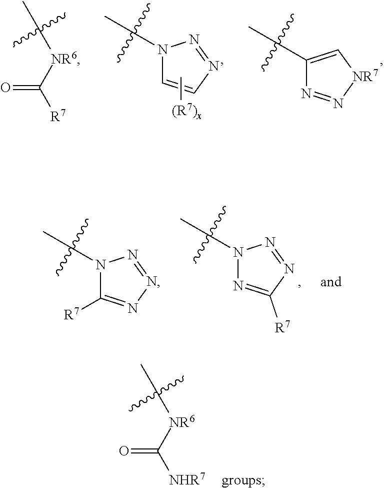
each R², which may be identical or different, is independently chosen from benzyl amino sulfonic acid and benzyl amino carboxylic acid groups;
each R³, which may be identical or different, is independently chosen from —CN, —CH₂CN, and —C(═O)Y¹ groups, wherein each Y¹, which may be identical or different, is independently chosen from C₁₋₈ alkyl, C₂₋₈ alkenyl, C₂₋₈ alkynyl, —OZ¹, —NHOH, —NHOCH₃, —NHCN, and —NZ¹Z² groups, wherein each Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, wherein Z¹ and Z² may join together along with the nitrogen atom to which they are attached to form a ring;
each R⁴, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
each R⁵, which may be identical or different, is independently chosen from —CN, C₁₋₈ alkyl, and C₁₋₄ haloalkyl groups;
each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups;
each R⁷, which may be identical or different, is independently chosen from H, C₁₋₈ alkyl, C₂₋₈ alkenyl, C₂₋₈ alkynyl, C₄₋₁₆ cycloalkylalkyl, 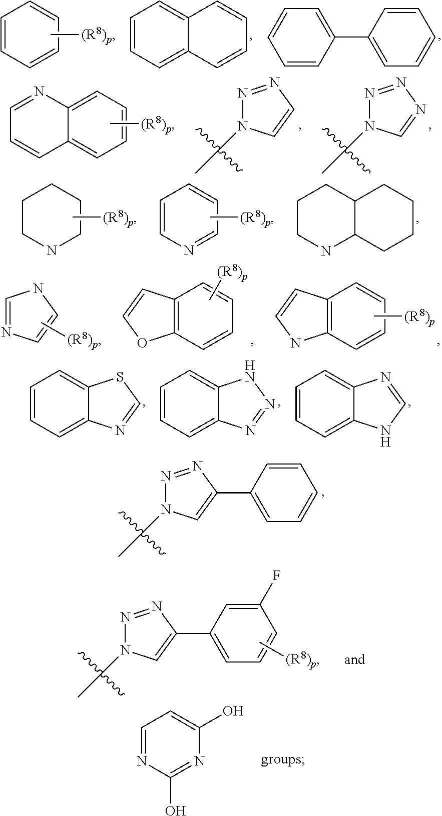
each R⁸, which may be identical or different, is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups;
each X¹, which may be identical or different, is independently chosen from —O— and —N(Z³)—, wherein each Z³, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
each j, which may be identical or different, is independently chosen from integers ranging from 1 to 29;
each k, which may be identical or different, is independently chosen from integers ranging from 1 to 10;
each n, which may be identical or different, is independently chosen from integers ranging from 1 to 10.
each p, which may be identical or different, is independently chosen from integers ranging from 0 to 3
each s, which may be identical or different, is independently chosen from integers ranging from 1 to 256;
each x, which may be identical or different, is independently chosen from integers ranging from 0 to 2;
m is chosen from integers ranging from 2 to 256; and
L is chosen from linker groups.

2. The at least one compound according to claim 1, wherein at least one R² is chosen from 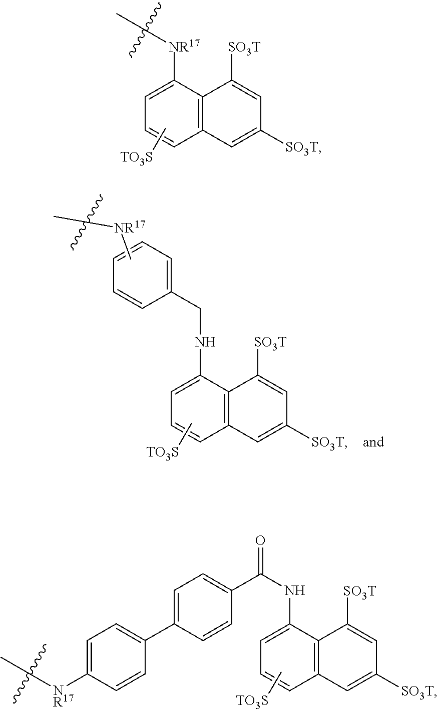
wherein
each R¹⁷, which may be identical or different, is independently chosen from H, C₁₋₈ alkyl, —C(═O)X³, and —C(═O)NHX³ groups;
each X³, which may be identical or different, is independently chosen from C₁₋₈ alkyl, C₂₋₈ alkenyl, C₂₋₈ alkynyl, C₆₋₁₈ aryl, and C₁₋₁₃ heteroaryl groups; and
each T, which may be identical or different, is independently chosen from H and pharmaceutically acceptable counterions.

3. The at least one compound according to claim 1, wherein at least one R² is chosen from

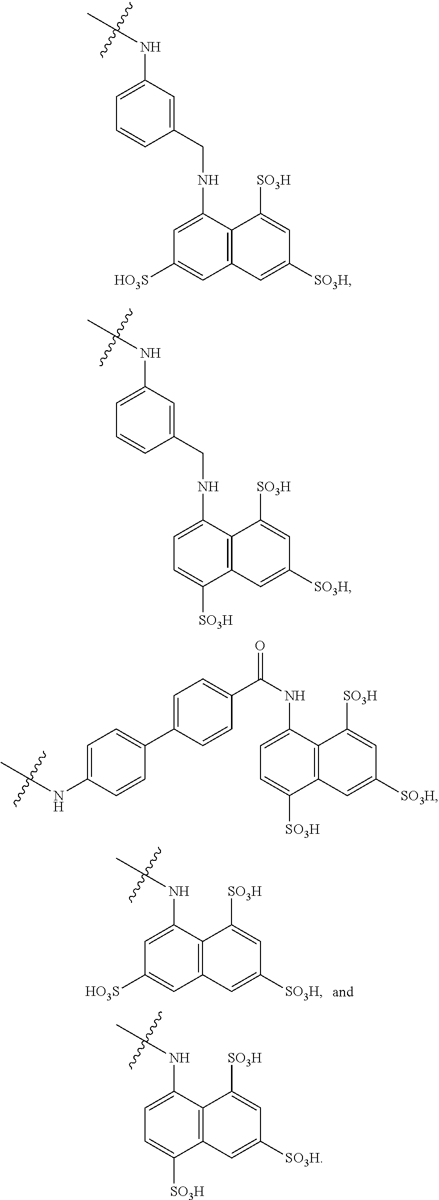 [SMILES: *Nc1cccc(CNc2cc(S(=O)(=O)O)cc3cc(S(=O)(=O)O)cc(S(=O)(=O)O)c23)c1 ; *Nc1cccc(CNc2ccc(S(=O)(=O)O)c3cc(S(=O)(=O)O)cc(S(=O)(=O)O)c23)c1 ; *Nc1ccc(-c2ccc(C(=O)Nc3ccc(S(=O)(=O)O)c4cc(S(=O)(=O)O)cc(S(=O)(=O)O)c34)cc2)cc1 ; *Nc1cc(S(=O)(=O)O)cc2cc(S(=O)(=O)O)cc(S(=O)(=O)O)c12 ; *Nc1ccc(S(=O)(=O)O)c2cc(S(=O)(=O)O)cc(S(=O)(=O)O)c12]

4. The at least one compound according to any preceding claim, wherein at least one R¹ is chosen from H, methyl, and ethyl.

5. The at least one compound according to claim 4, wherein at least one R¹ is methyl.

6. The at least one compound according to claim 4, wherein at least one R¹ is ethyl.

7. The at least one compound according to any of claims 1-3, wherein at least one R¹ is chosen from

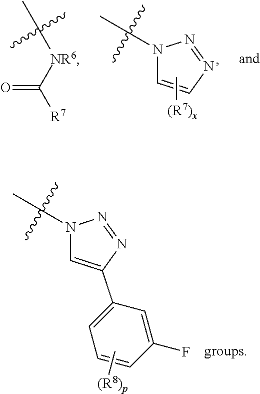

8. The at least one compound according to any of claims 1-3, wherein at least one R¹ is chosen from

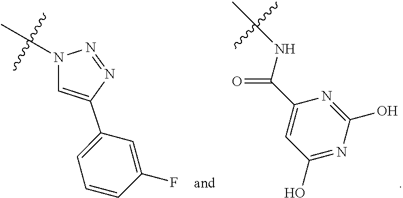 [SMILES: *n1cc(-c2cccc(F)c2)nn1 ; "and" ; *NC(=O)c1cc(O)nc(O)n1 ; "."]

9. The at least one compound according to claim 8, wherein at least one R¹ is

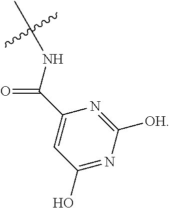 [SMILES: *NC(=O)c1cc(O)nc(O)n1]

10. The at least one compound according to any preceding claim, wherein at least one R³ is chosen from —C(═O)Y¹ groups.

11. The at least one compound according to claim 10, wherein at least one R³ is chosen from

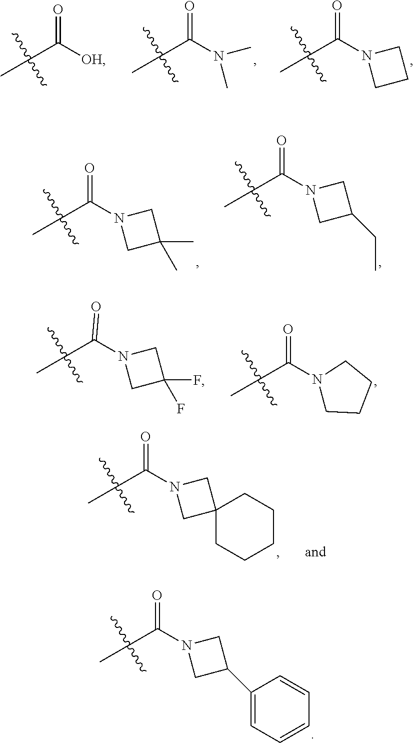

12. The at least one compound according to claim 11, wherein at least one R³ is chosen from

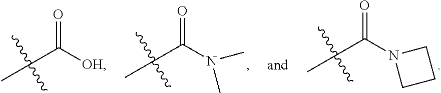

13. The at least one compound according to claim 12, wherein at least one R³ is

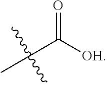 [SMILES: *C(=O)O]

14. The at least one compound according to any preceding claim, wherein at least one R⁴ is chosen from C₁₋₈ alkyl and C₄₋₁₆ cycloalkylalkyl groups.

15. The at least one compound according to claim 14, wherein at least one R⁴ is chosen from propyl and cyclohexylmethyl.

16. The at least one compound according to claim 14, wherein at least one R⁴ is cyclohexylmethyl.

17. The at least one compound according to any preceding claim, wherein at least one R⁵ is chosen from —CN, CF₃, and methyl.

18. The at least one compound according to claim 17, wherein at least one R⁵ is methyl.

19. The at least one compound according to any preceding claim, wherein at least one X¹ is chosen from —O— and —NH—.

20. The at least one compound according to claim 19, wherein at least one X¹ is —O—.

21. The at least one compound according to claim 19, wherein at least one X¹ is —NH—.

22. The at least one compound according to any preceding claim, wherein at least one j is chosen from integers ranging from 1 to 10.

23. The at least one compound according to any preceding claim, wherein at least one j is chosen from integers ranging from 1 to 5.

24. The at least one compound according to any preceding claim, wherein at least one j is 1.

25. The at least one compound according to any preceding claim, wherein at least one k is chosen from integers ranging from 1 to 7.

26. The at least one compound according to any preceding claim, wherein at least one k is chosen from integers ranging from 1 to 5.

27. The at least one compound according to any preceding claim, wherein at least one k is 1.

28. The at least one compound according to any preceding claim, wherein at least one n is chosen from integers ranging from 1 to 7.

29. The at least one compound according to any preceding claim, wherein at least one n is chosen from integers ranging from 1 to 5.

30. The at least one compound according to any preceding claim, wherein at least one n is 1.

31. The at least one compound according to claim 1 chosen from compounds having the following Formula: 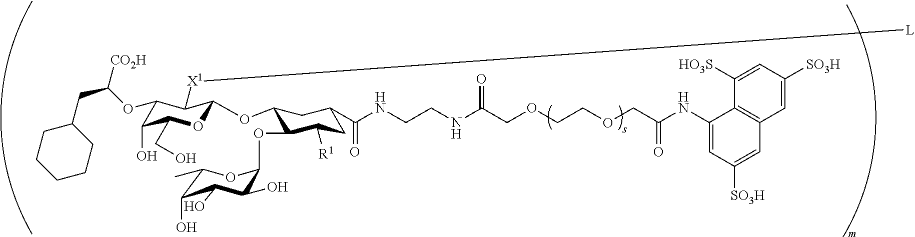
and pharmaceutically acceptable salts thereof.

32. The at least one compound according to claim 31 chosen from compounds having the following Formulae: 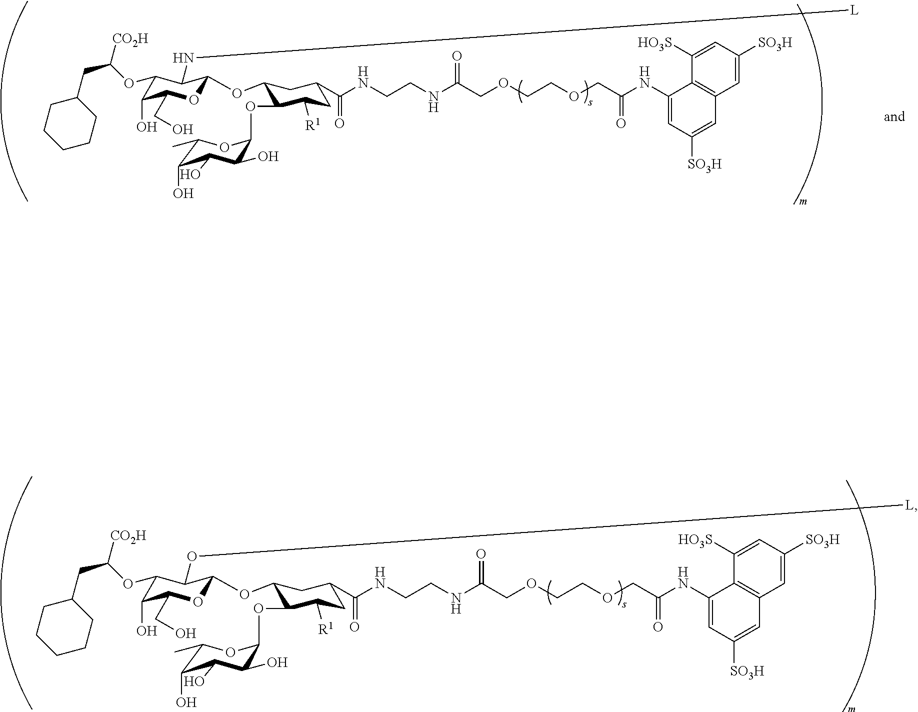
and pharmaceutically acceptable salts of any of the foregoing.

33. The at least one compound according to claim 31 chosen from compounds having the following Formulae: 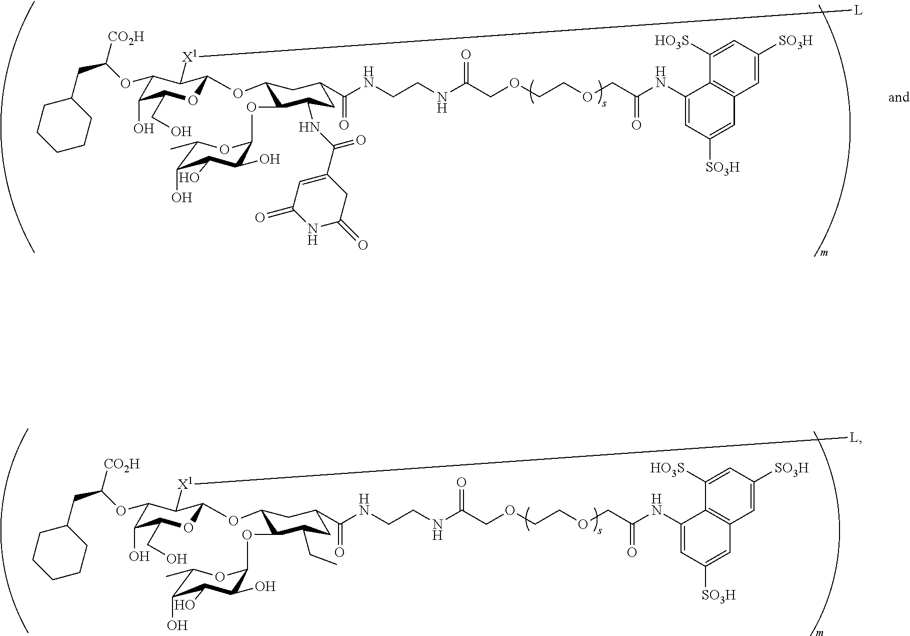
and pharmaceutically acceptable salts of any of the foregoing.

34. The at least one compound according to claim 31 chosen from compounds having the following Formulae: 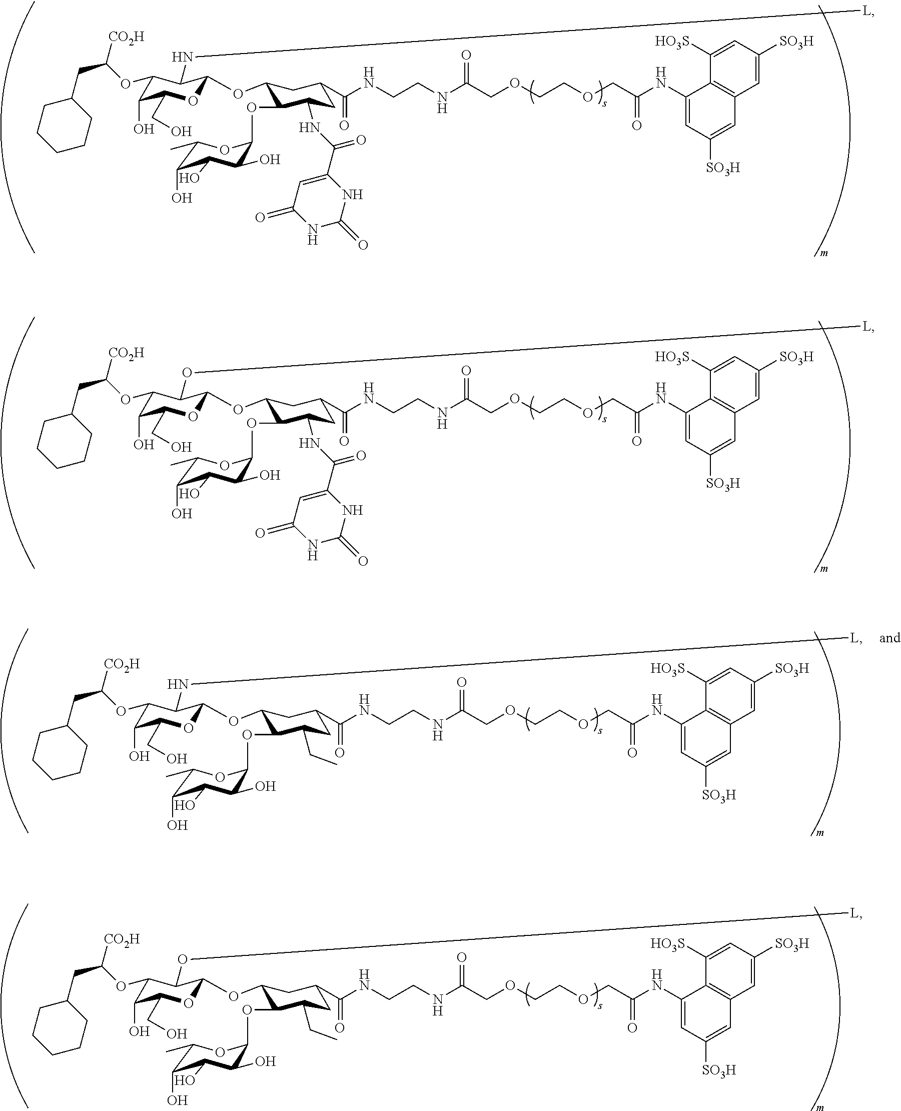
and pharmaceutically acceptable salts of any of the foregoing.

35. The at least one compound according to any preceding claim, wherein at least one s is chosen from integers ranging from 1 to 32.

36. The at least one compound according to any preceding claim, wherein at least one s is chosen from integers ranging from 1 to 16.

37. The at least one compound according to any preceding claim, wherein at least one s is chosen from integers ranging from 1 to 8.

38. The at least one compound according to any preceding claim, wherein at least one s is chosen from integers ranging from 1 to 4.

39. The at least one compound according to any preceding claim, wherein at least one s is 1.

40. The at least one compound according to any preceding claim, wherein m is chosen from integers ranging from 2 to 128.

41. The at least one compound according to any preceding claim, wherein m is chosen from integers ranging from 2 to 32.

42. The at least one compound according to any preceding claim, wherein m is chosen from integers ranging from 2 to 16.

43. The at least one compound according to any preceding claim, wherein m is chosen from integers ranging from 2 to 8.

44. The at least one compound according to claim 43, wherein L is chosen from:

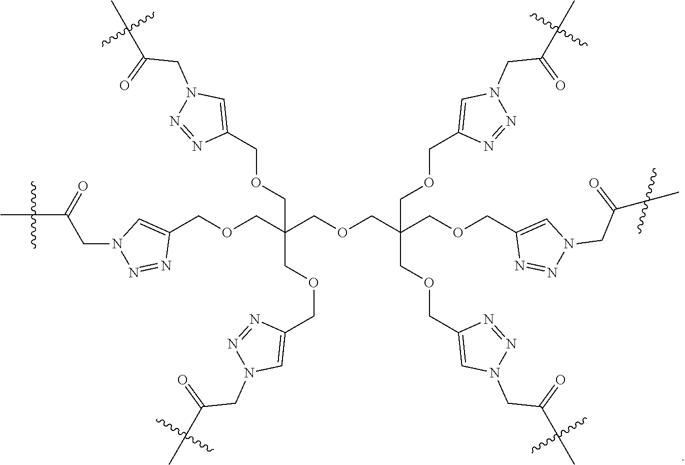 [SMILES: *C(=O)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

45. The at least one compound according to any of claims 1-43, wherein m is chosen from integers ranging from 2 to 4.

46. The at least one compound according to claim 45, wherein m is 4.

47. The at least one compound according to claim 46, wherein L is chosen from:

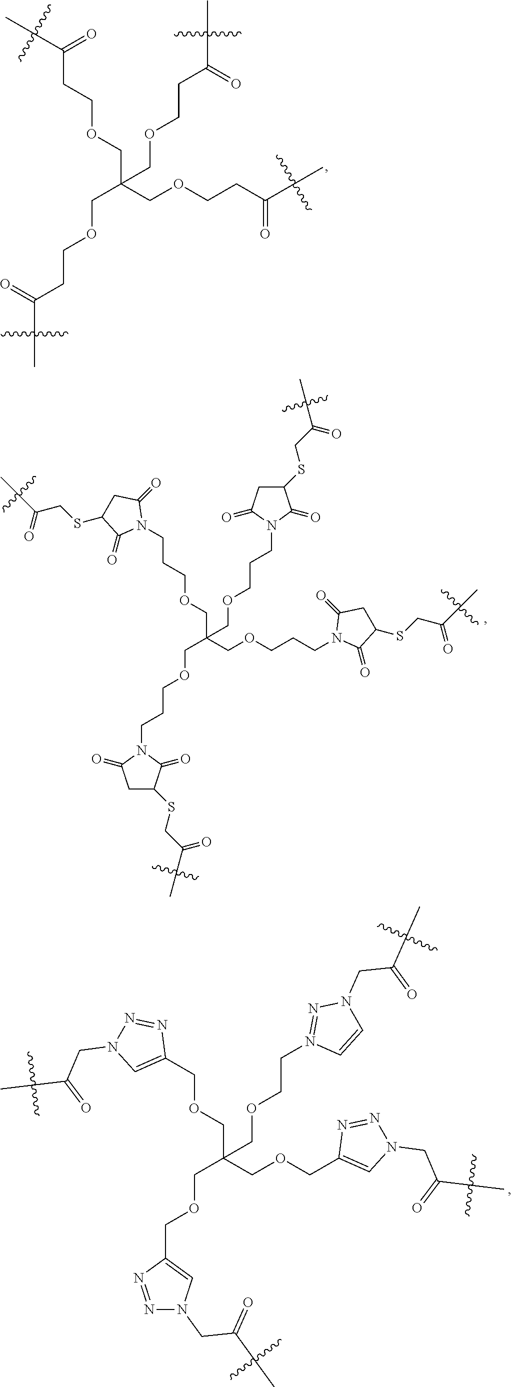

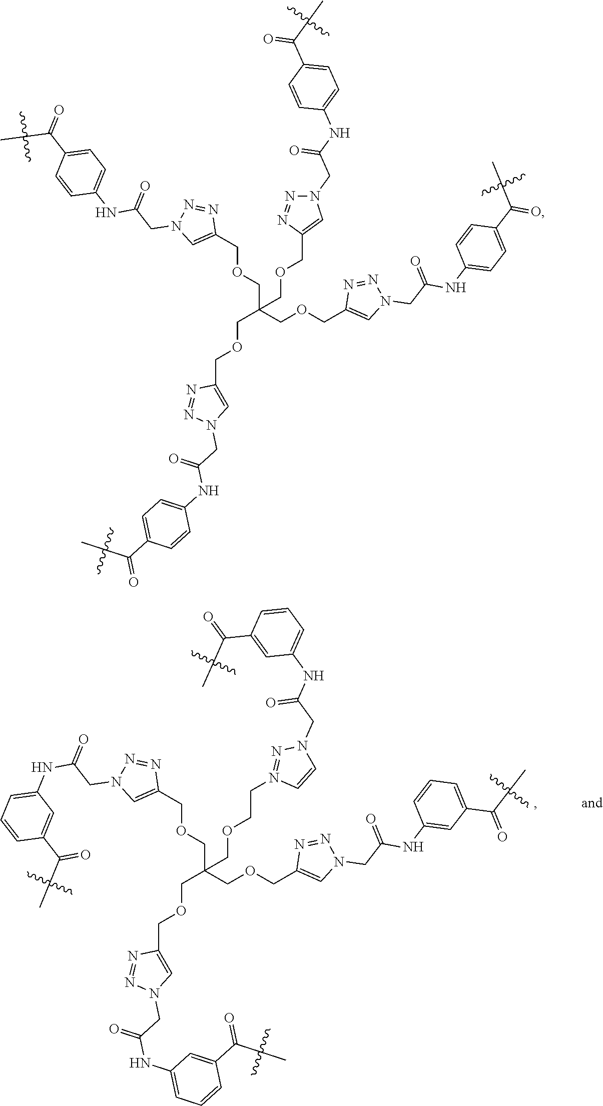

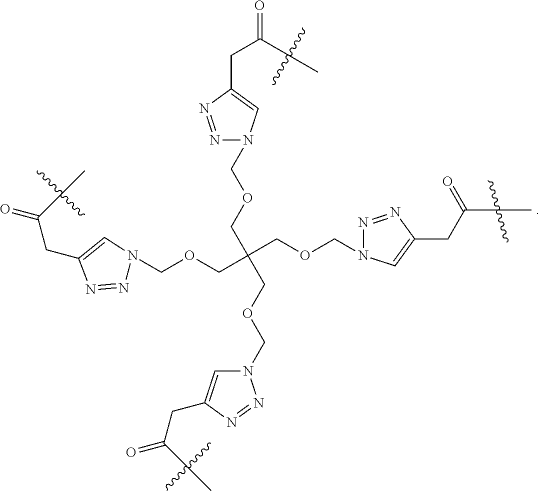 [SMILES: *C(=O)Cc1cn(COCC(COCn2cc(CC(*)=O)nn2)(COCn2cc(CC(*)=O)nn2)COCn2cc(CC(*)=O)nn2)nn1]

48. The at least one compound according to claim 45, wherein m is 3.

49. The at least one compound according to claim 48, wherein L is chosen from:

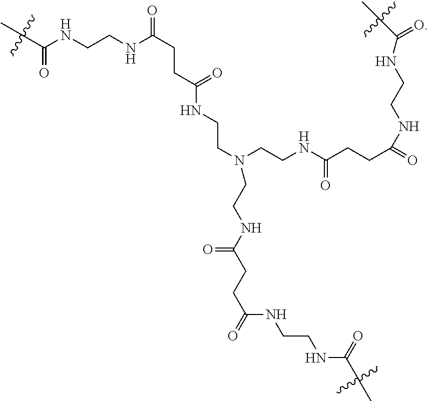 [SMILES: *C(=O)NCCNC(=O)CCC(=O)NCCN(CCNC(=O)CCC(=O)NCCNC(*)=O)CCNC(=O)CCC(=O)NCCNC(*)=O]

50. The at least one compound according to claim 45, wherein m is 2.

51. The at least one compound according to claim 50, wherein L is chosen from 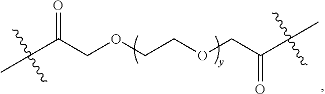
wherein y is chosen from integers ranging from 0 to 250.

52. The at least one compound according to claim 50, wherein L is chosen from 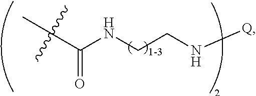
wherein Q is chosen from 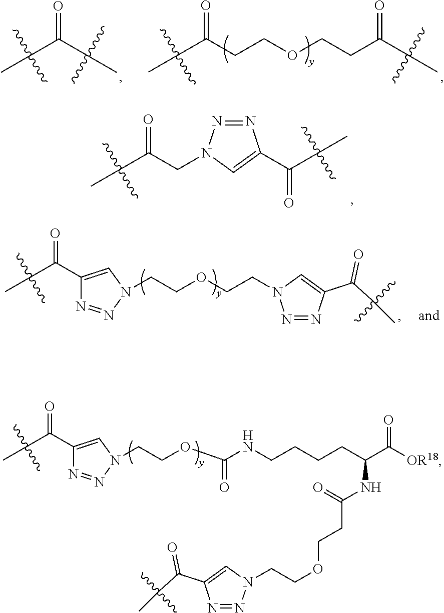
wherein R¹⁸ is chosen from H, C₁₋₈ alkyl, C₆₋₁₈ aryl, C₇₋₁₉ arylalkyl, and C₁₋₁₃ heteroaryl groups and each y, which may be identical or different, is independently chosen from integers ranging from 0 to 250.

53. The at least one compound according to claim 50, wherein L is chosen from 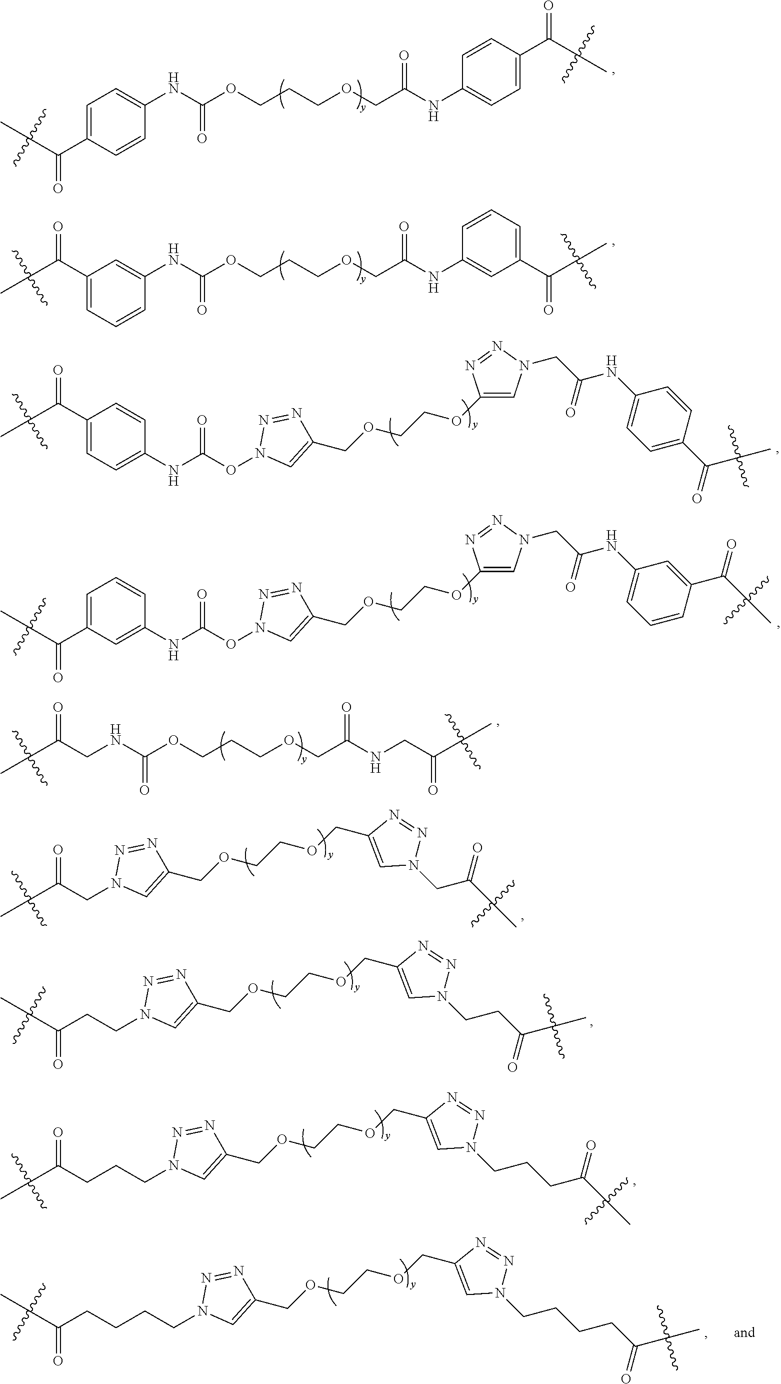 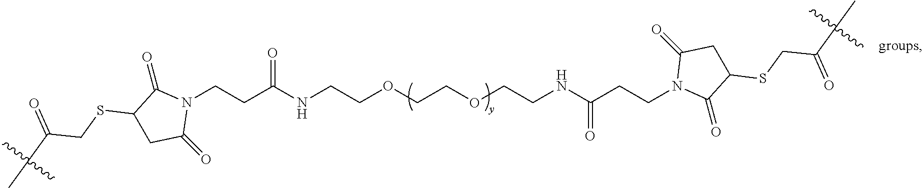
wherein y is chosen from integers ranging from 0 to 250.

54. The at least one compound according to claim 50, wherein L is chosen from: 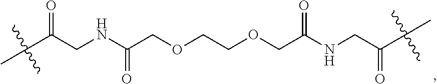
wherein y is chosen from integers ranging from 0 to 250.

55. The at least one compound according to any of claim 50, wherein L is chosen from:

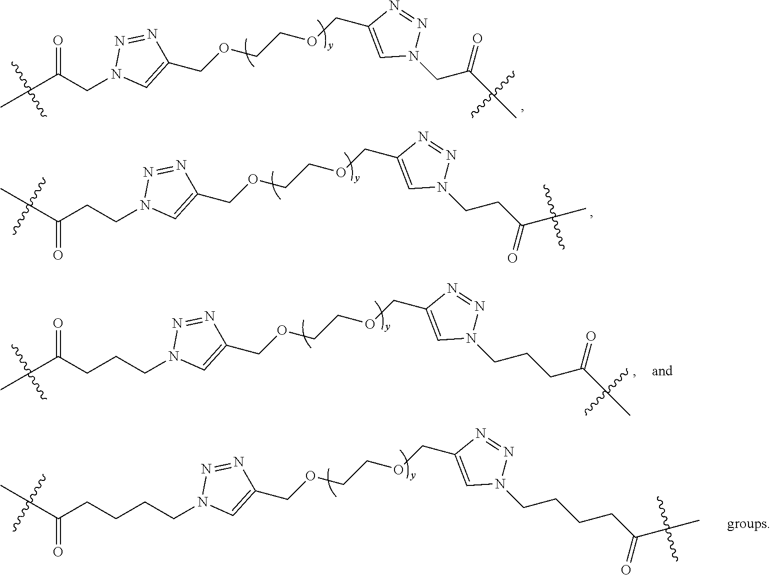

56. The at least one compound according to claim 50, wherein L is chosen from:

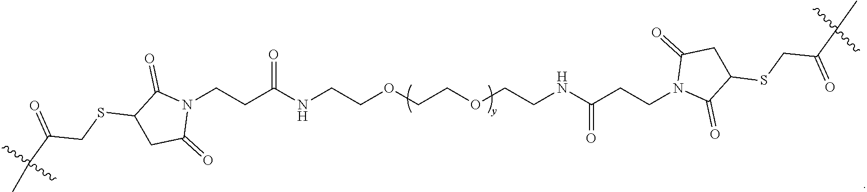

57. The at least one compound according to any of claims 51-56, wherein each y is identical and chosen from integers ranging from 0 to 25.

58. The at least one compound according to claim 57, wherein each y is identical and chosen from integers ranging from 0 to 20.

59. The at least one compound according to claim 57, wherein each y is identical and chosen from integers ranging from 0 to 15.

60. The at least one compound according to claim 57, wherein each y is 14.

61. The at least one compound according to claim 57, wherein each y is 10.

62. The at least one compound according to claim 57, wherein each y is 3.

63. The at least one compound according to claim 57, wherein each y is 2.

64. The at least one compound according to claim 57, wherein each y is 1.

65. The at least one compound according to any preceding claim, wherein each R¹ is identical, each R² is identical, each R³ is identical, each R⁴ is identical, each R⁵ is identical, each X¹ is identical, each j is identical, each k is identical, each n is identical, and each s is identical.

66. A composition comprising at least one compound according to any preceding claim and at least one additional pharmaceutically acceptable ingredient.

67. A method for modulating a selectin-mediated function comprising administering to a subject in need thereof an effective amount of at least one compound of any of claims 1-65 and optionally at least one additional pharmaceutically acceptable ingredient.

68. The method of claim 67, wherein the selectin-mediated function is selectin-mediated intercellular adhesion.

69. The method of claim 67 or 68, wherein the selectin-mediated function is inhibited.

70. A method for contacting a cell expressing a selectin to modulate the selectin's function comprising administering to a subject in need thereof an effective amount of at least one compound of any of claims 1-65 and optionally at least one additional pharmaceutically acceptable ingredient.

71. A method for inhibiting the development of a condition associated with an excessive selectin-mediated function comprising administering to a subject in need thereof an effective amount of at least one compound of any of claims 1-65 and optionally at least one additional pharmaceutically acceptable ingredient.

72. A method for inhibiting rejection of a transplanted tissue comprising administering to a subject in need thereof an effective amount of at least one compound of any of claims 1-65 and optionally at least one additional pharmaceutically acceptable ingredient.

73. A method for treating sickle cell disease or complications associated therewith comprising administering to a subject in need thereof an effective amount of at least one compound of any of claims 1-65 and optionally at least one additional pharmaceutically acceptable ingredient.

74. A method for treating vaso-occlusive crisis comprising administering to a subject in need thereof an effective amount of at least one compound of any of claims 1-65 and optionally at least one additional pharmaceutically acceptable ingredient.

75. A method for treating graft versus host disease or complications associated therewith comprising administering to a subject in need thereof an effective amount of at least one compound of any of claims 1-65 and optionally at least one additional pharmaceutically acceptable ingredient.

76. A method for treating sinusoidal obstruction syndrome or complications associated therewith comprising administering to a subject in need thereof an effective amount of at least one compound of any of claims 1-65 and optionally at least one additional pharmaceutically acceptable ingredient.

77. A method for treating a cancer of the blood and complications associated therewith comprising administering to a subject in need thereof an effective amount of at least one compound of any of claims 1-65 and optionally at least one additional pharmaceutically acceptable ingredient.

78. The method of claim 77, wherein the cancer of the blood is chosen from acute myelogenous leukemia, acute lymphoblastic leukemia, chronic myelogenous leukemia, and multiple myeloma.

79. A method for treating epilepsy comprising administering to a subject in need thereof an effective amount of at least one compound of any of claims 1-65 and optionally at least one additional pharmaceutically acceptable ingredient.
